(19) United States
(12) Patent Application Publication
(10) Pub. No.: US 2022/0160337 A1
(43) Pub. Date: May 26, 2022

(54) MICROBIAL COLLECTION FORMULATIONS, DEVICES AND SYSTEMS

(71) Applicant: Anaerobe Systems, Morgan Hill, CA (US)

(72) Inventor: Steven M. COX, Morgan Hill, CA (US)

(21) Appl. No.: 17/543,308

(22) Filed: Dec. 6, 2021

Related U.S. Application Data
(63) Continuation of application No. PCT/US2020/036457, filed on Jun. 5, 2020.
(60) Provisional application No. 63/025,709, filed on May 15, 2020.
(60) Provisional application No. 62/858,850, filed on Jun. 7, 2019.

(51) Int. Cl. A61B 10/00; B01L 3/00; A01N 1/02

(52) U.S. Cl. CPC A61B 10/0038; B01L 3/50825; A61B 10/0096; A01N 1/0226; B01L 2300/0832; B01L 2200/16; B01L 2300/021; B01L 2300/042; B01L 2200/0689

(57) ABSTRACT

The present disclosure provides devices, systems, kits, methods, and compositions for collecting, storing, transporting, preserving, and culturing anaerobic microorganisms obtained from a subject or a biological sample of the subject (e.g., a human subject).

### CROSS-REFERENCE

This application is a bypass continuation application of PCT/US2020/036457, filed Jun. 5, 2020, which claims the benefit of U.S. Provisional Application Nos. 63/025,709, filed May 15, 2020, and 62/858,850, filed Jun. 7, 2019, which applications are incorporated herein by reference in their entirety for all purposes.

### BACKGROUND

The human microbiome plays a fundamental role in the human immune system with a strong relationship to health and disease. Microbiota collected from a host (e.g., a stool sample of an animal), when coupled with host data, can have several uses including, but not limited to, developing animal models, diagnosing medical conditions, and building organism collections or microbial compositions for use in biotherapeutics, probiotics, or for other research purposes.

Most of the microbiota can be found in the gut and is made up primarily of anaerobic microorganisms, both strict and facultatively anaerobic. A large portion of the research and analysis of the microbiome to date is based around 16S ribosomal ribonucleic acid (RNA) (i.e., rRNA) sequencing to identify the organism composition, population and distribution. These sequencing methods can allow the identification of organisms present in a given microbial population, but they may not allow recovery and/or growth of any organisms of interest. Thus, conventional sample collection may preserve the nucleic acids (e.g., deoxyribonucleic acid (DNA), RNA, etc.) but may not preserve the organism's (e.g., a bacterium's) viability. Sequencing also has a limitation in that it does not identify if the organisms are actively growing at the site from where the sample is obtained (e.g., the human gut), or if their genetic material stems from another area of the body (e.g., the skin).

The collection and successful culture of organisms from biological samples (e.g., microbiota samples), such as feces, can pose a challenge. Biological samples of the present disclosure can comprise aerobic and anaerobic microorganisms (e.g., obligate anaerobic organisms). Obligate anaerobic organisms can rapidly lose their viability in the presence of atmospheric oxygen. Those organisms lack specific enzymes that can neutralize toxic metabolic byproducts such as hydrogen peroxide, superoxide anions, and hydroxyl radicals produced in the presence of oxygen. Other metabolic byproducts, including short chain fatty acids and alcohols, can impact the viability of microorganisms in the sample if metabolism and growth continues after collection. Moreover, the growth and proliferation of some fast-growing organisms in a sample can outcompete and prevent the recovery of other, more fastidious and slow growing organisms.

Collection of a biological sample in the laboratory where the recovery and culture of microorganisms will be performed within a matter of hours greatly reduces the amount of time during which these organisms may be exposed to oxygen and accumulating metabolites, and thus may allow those organisms to continue their metabolism and growth. This, however, can significantly limit the accessible population for sample collection to those subjects within immediate vicinity to a laboratory and with the ability to provide a sample in the laboratory setting. Samples can also be frozen after sample collection, which can stop metabolic processes and can preserve the organisms in stasis. However, freezing poses several challenges to the person providing the sample. Moreover, freezing organisms (e.g., bacteria) without a cryoprotectant can damage cell membranes. Biological samples can typically be collected from the general population outside the laboratory where freezing may not be feasible or convenient. Thus, samples may not be frozen in a timely manner and/or sent back to the lab in a timely manner. Shipping of frozen samples requires expensive overnight shipping, special insulated packaging, and dry ice necessary to keep the samples frozen may increase costs and logistics. Samples collected later in the day may not be sent back until the following day, and samples shipped or mailed at the end of a week may experience longer transit times of several days, e.g., over a weekend.

### SUMMARY

Recognized herein is a need for a practical and user-friendly system of collecting, preserving, and shipping biological samples comprising human microbiota (e.g., anaerobic microorganisms), such as, for example, for the purpose of culturing those organisms and storing the samples in a laboratory.

In various aspects, the present disclosure provides kits, methods, systems, and instructions that can allow for collection, transport, storage, analysis, and growth of microorganisms obtained from a biological sample. The kits, methods, and systems described herein can maintain the viability of such microorganisms by e.g., reducing and/or maintaining a low (e.g., lower than atmospheric oxygen) oxygen content in the collection/preservation formulation and collection container (e.g., a tube, vial, petri dish, etc., made out of e.g., plastic and/or glass), and/or by removing, or preventing the accumulation of, toxic metabolites that can be produced by microorganisms during transport and/or storage. The kits, methods and systems provided herein also allow culturing of microorganisms of a biological sample following storage of the biological sample in a formulation described herein for a certain time period of several days or weeks (e.g., 1, 2, 3, 4, 5, 6, or more days).

In various aspects, the present disclosure provides a kit for obtaining a stool sample comprising at least one anaerobic microorganism from a subject and preserving the at least one anaerobic microorganism, comprising: a formulation comprising an antioxidant, at least one oxygen scavenger and a metabolite scavenger; a container configured to store the stool sample in the formulation, which container is configured to be sealed; a collection device configured to obtain the stool sample from the subject or a biological sample of the subject; and instructions that direct a user to (i) use the collection device to obtain the stool sample from the subject, (ii) store the stool sample in the container having the formulation, and (iii) seal the container comprising the stool sample and formulation, wherein the container and formulation are configured to provide a concentration of oxygen gas in the container, subsequent to sealing, at less than 20.95% for a time period of at least 2 days as measured at 25° C. In some aspects, the formulation further comprises an electrolyte. In some aspects, the electrolyte comprises sodium chloride, potassium chloride, sodium phosphate, magnesium sulfate, or any combination thereof. In some aspects, the antioxidant is selected from the group consisting of a peroxidase, an ascorbic acid, a glutathione, a dithiothreitol, a derivative thereof, or any combination thereof. In some aspects, the at least one oxygen scavenger comprises sodium thioglycolate, a cysteine, a peroxidase, a catalase, a dismutase, or any combination thereof. In some aspects, the at least one oxygen scavenger comprise at least two oxygen scavengers, at least three oxygen scavengers, at least four oxygen scavengers, at least five oxygen scavengers, at least six oxygen scavengers, at least seven oxygen scavengers, at least eight oxygen scavengers, at least nine oxygen scavengers, or at least ten oxygen scavengers. In some aspects, the metabolite scavenger comprises activated carbon. In some aspects, the time period is at least 4 days. In some aspects, the time period is at least 5 days. In some aspects, the kit further comprises a homogenizing medium that homogenizes a solution comprising the stool sample and the formulation. In some aspects, the formulation has a volume from about 5 mL to about 15 mL. In some aspects, the formulation has a volume of at least about 9 mL. In some aspects, a dilution ratio of stool sample to formulation in the container is from about 1:20 to about 1:5. In some aspects, the dilution ratio of stool sample to formulation in the container is at least about 1:10. In some aspects, the collection device is a scoop, a spatula, or a brush.

In various aspects, the present disclosure provides a method for obtaining a stool sample comprising at least one anaerobic microorganism from a subject and preserving the at least one anaerobic microorganism, comprising: (a) using a collection device to obtain the stool sample from the subject or a biological sample of the subject; (b) storing the stool sample obtained in (a) in a container comprising a formulation, which formulation comprises an antioxidant, at least one oxygen scavenger and a metabolite scavenger; and (c) sealing the container comprising the stool sample and formulation, wherein a concentration of oxygen gas in the container, subsequent to sealing, is less than 20.95% for a time period of at least 2 days as measured at 25° C. In some aspects, the formulation further comprises an electrolyte. In some aspects, the electrolyte comprises sodium chloride, potassium chloride, sodium phosphate, magnesium sulfate, or any combination thereof. In some aspects, the antioxidant is selected from the group consisting of a peroxidase, an ascorbic acid, a glutathione, a dithiothreitol, a derivative thereof, or any combination thereof. In some aspects, the at least one oxygen scavenger comprises sodium thioglycolate, a cysteine, a peroxidase, a catalase, a dismutase, or any combination thereof. In some aspects, the at least one oxygen scavenger comprise at least two oxygen scavengers, at least three oxygen scavengers, at least four oxygen scavengers, at least five oxygen scavengers, at least six oxygen scavengers, at least seven oxygen scavengers, at least eight oxygen scavengers, at least nine oxygen scavengers, or at least ten oxygen scavengers. In some aspects, the metabolite scavenger comprises activated carbon. In some aspects, the time period is at least 4 days. In some aspects, the time period is at least 5 days. In some aspects, the method further comprises, subsequent to (c), homogenizing the stool sample and the formulation. In some aspects, the homogenizing comprises shaking or vortexing the formulation comprising the stool sample. In some aspects, the method further comprises, subsequent to (c), depositing the container with a processing unit that processes the stool sample or derivative thereof. In some aspects, the depositing comprises providing the container to a parcel delivery service that delivers the container to the processing unit. In some aspects, the method further comprises, prior to (a), receiving a kit comprising one or more of the formulation, the container and the collection device. In some aspects, the kit comprises two or more of the formulation, the container and the collection device. In some aspects, the kit comprises the formulation, the container and the collection device. In some aspects, the at least one anaerobic microorganism comprises at least one anaerobic bacterial species. In some aspects, preserving the at least one anaerobic microorganism comprises preserving a viability of the at least one anaerobic microorganism. In some aspects, the formulation has a volume from about 5 mL to about 15 mL. In some aspects, the formulation has a volume of at least about 9 mL. In some aspects, a dilution ratio of stool sample to formulation in the container is from about 1:20 to about 1:5. In some aspects, the dilution ratio of stool sample to formulation in the container is at least about 1:10. In some aspects, the collection device is a scoop, a spatula, or a brush.

In some aspects, the present disclosure provides a system for obtaining a stool sample comprising at least one anaerobic microorganism from a subject and preserving the at least one anaerobic microorganism, comprising (i) a formulation comprising an antioxidant, at least one oxygen scavenger and a metabolite scavenger, (ii) a container configured to store the stool sample in the formulation, which container is configured to be sealed, and (iii) a collection device configured to obtain the stool sample from the subject or a biological sample of the subject, wherein the container and the formulation are configured to provide a concentration of oxygen gas in the container at less than 20.95% for a time period of at least 2 days as measured at 25° C. when the container having the formulation is sealed. In some aspects, the formulation further comprises an electrolyte. In some aspects, the electrolyte comprises sodium chloride, potassium chloride, sodium phosphate, magnesium sulfate, or any combination thereof. In some aspects, the antioxidant is selected from the group consisting of a peroxidase, an ascorbic acid, a glutathione, a dithiothreitol, a derivative thereof, or any combination thereof. In some aspects, the at least one oxygen scavenger comprises sodium thioglycolate, a cysteine, a peroxidase, a catalase, a dismutase, or any combination thereof. In some aspects, the at least one oxygen scavenger comprise at least two oxygen scavengers, at least three oxygen scavengers, at least five oxygen scavengers, or at least ten oxygen scavengers. In some aspects, the metabolite scavenger comprises activated carbon. In some aspects, the time period is at least 4 days. In some aspects, the time period is at least 5 days. In some aspects, the system further comprises a homogenizing medium that homogenizes a solution comprising the stool sample and the formulation. In some aspects, the formulation has a volume from about 5 mL to about 15 mL. In some aspects, the formulation has a volume of at least about 9 mL. In some aspects, a dilution ratio of stool sample to formulation in the container is from about 1:20 to about 1:5. In some aspects, the dilution ratio of stool sample to formulation in the container is at least about 1:10. In some aspects, the collection device is a scoop, a spatula, or a brush.

Another aspect of the present disclosure provides liquid collection formulations, tubes, and/or containers that can maintain the viability of microorganisms (e.g., anaerobic microorganisms) of a biological sample. Thus, the presently described kits, devices, systems, methods and compositions can allow for analysis (e.g., ribosomal ribonucleic acid (rRNA)/16S sequencing) and further growth of microorganisms of a biological sample (e.g., as compared to solely preserving the genetic material of the organisms in conventional approaches). This may allow a user to analyze and identify the microorganisms present in a biological sample, and it may allow the user to further culture and grow such organisms due to their maintained viability during transport from the sampling site to the analysis/processing facility (e.g., a site where a diagnostic test or assay can be conducted). Moreover, the herein described methods, compositions, and kits may allow the preservation of the microorganisms' viability under practical and user-friendly conditions. Such practical and user-friendly conditions may include sample collection and storage at room temperature (e.g., without freezing) and sample transport using regular transit times (e.g., without rush transport and associated limitations) due to efficient preservation of the microorganism's viability for at least 2, 3, 4, 5, 6, or more days.

In various aspects, the present disclosure provides a kit for storing a biological sample comprising at least one microorganism from a subject, comprising: a formulation comprising an antioxidant and an oxygen scavenger; a container configured to store the biological sample and the formulation, which container is configured to be sealed; and instructions that direct a user to (i) dispose the formulation and the biological sample in the container, and (ii) seal the container comprising the formulation and the biological sample, to thereby preserve a viability of the at least one microorganism for a time period of at least 2 days. In some aspects, the formulation is a liquid, and wherein the container comprises the formulation and a head space filled with a gas, wherein the gas comprises less than about 20% oxygen. In some aspects, the antioxidant comprises ascorbic acid, glutathione, dithiothreitol, glutathione peroxidase, a peroxidase, superoxide dismutase, catalase, a derivative thereof, or any combination thereof. In some aspects, the oxygen scavenger comprises thioglycolate, cysteine, glutathione, glutathione peroxidase, peroxidase, catalase, superoxide dismutase, or any combination thereof. In some aspects, the formulation further comprises a cryoprotectant. In some aspects, the cryoprotectant comprises glycerol. In some aspects, comprising a collection device configured to obtain the biological sample from the subject. In some aspects, the collection device is a scoop, a spatula, or a brush. In some aspects, the formulation further comprises an electrolyte. In some aspects, the electrolyte comprises sodium chloride, potassium chloride, sodium phosphate, magnesium sulfate, or any combination thereof. In some aspects, the formulation further comprises a metabolite scavenger. In some aspects, the metabolite scavenger comprises activated carbon, glutathione, ascorbic acid, or a combination thereof. In some aspects, the formulation comprises at least two oxygen scavengers comprising the oxygen scavenger. In some aspects, the antioxidant is present in the formulation in an amount ranging from about 10⁻¹⁰ moles per liter (mol/L) to about 10 mol/L. In some aspects, the oxygen scavenger is present in the formulation in an amount ranging from about 10⁻¹⁰ mol/L to about 10 mol/L. In some aspects, the cryoprotectant is present in the formulation in an amount ranging from about 10⁻¹⁰ mol/L to about 10 mol/L. In some aspects, the electrolyte is present in the formulation in an amount ranging from about 10⁻¹⁰ mol/L to about 10 mol/L. In some aspects, the metabolite scavenger is present in the formulation in an amount ranging from about 10⁻¹⁰ mol/L to about 10 mol/L. In some aspects, the instructions direct the user to use the collection device to obtain the biological sample from the subject. In some aspects, the instructions direct the user to subject the container comprising the formulation and the biological sample to conditions sufficient to generate a homogeneous mixture comprising the formulation and the biological sample. In some aspects, the formulation has a volume from about 5 milliliters (mL) to about 15 mL. In some aspects, the formulation has a volume of at least about 9 mL. In some aspects, the formulation is configured to preserve the viability of the at least one microorganism at an abundance of least about 70% of the at least one microorganism as compared to an abundance of the at least one microorganism in a fresh biological sample. In some aspects, the formulation is configured to preserve the viability of the at least one microorganism at an abundance of least about 80% of the at least one microorganism as compared to an abundance of the at least one microorganism in a fresh biological sample. In some aspects, the formulation is configured to preserve the viability of the at least one microorganism at an abundance of least about 90% of the at least one microorganism as compared to an abundance of the at least one microorganism in a fresh biological sample. In some aspects, the abundance is determined by culturing and sequencing the at least one microorganism. In some aspects, the at least one microorganism comprises at least one anaerobic microorganism. In some aspects, the at least one anaerobic microorganism is an obligate anaerobic microorganism or a facultative anaerobic microorganism. In some aspects, the at least one microorganism belongs to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp. In some aspects, the formulation is configured to preserve a viability of at least two microorganisms, which at least two microorganisms comprise the at least one microorganism. In some aspects, the formulation is configured to preserve a viability of at least five microorganisms, which at least five microorganisms comprise the at least one microorganism. In some aspects, the formulation is configured to preserve a viability of at least ten microorganisms, which at least ten microorganisms comprise the at least one microorganism. In some aspects, the formulation is configured to preserve a viability of at least 80% of the at least one microorganism present in the biological sample. In some aspects, the formulation is configured to preserve a viability of at least 90% of the at least one microorganism present in the biological sample. In some aspects, the formulation is configured to preserve a viability of at least 93% of the at least one microorganism present in the biological sample. In some aspects, the formulation is configured to preserve a viability of at least 80% of the at least one microorganism, when the at least one microorganism is present in the biological sample with a relative abundance of equal to or lower than about 0.01%. In some aspects, the formulation is configured to preserve a viability of at least 90% of the at least one microorganism, when the at least one microorganism is present in the biological sample with a relative abundance of equal to or lower than about 0.01%. In some aspects, the biological sample is a stool sample.

In various aspects, provided herein is a method for forming a container for preserving at least one microorganism, comprising: (a) providing the container comprising a formulation comprising an antioxidant and an oxygen scavenger; and (b) sealing the container; wherein (a) and (b) are performed under anaerobic conditions, wherein the container comprising the formulation is configured to preserve the at least one microorganism for a time period of at least 2 days. In some aspects, the anaerobic conditions comprise an oxygen gas content of at most about 20%.

In various aspects, provided herein is a method for preserving a biological sample comprising at least one microorganism, the method comprising: (a) providing a container comprising (i) the biological sample and (ii) a formulation comprising an antioxidant and an oxygen scavenger; and (b) sealing the container, to thereby preserve a viability of the at least one microorganism for a time period of at least 2 days. In some aspects, the method further comprises preserving the viability of the at least one microorganism for a time period of at least 3 days. In some aspects, the antioxidant and oxygen scavenger are different compounds. In some aspects, the method further comprises preserving the viability of the at least one microorganism for a time period of at least 4 days. In some aspects, the method further comprises preserving the viability of the at least one microorganism for a time period of at least 5 days. In some aspects, the method further comprises, subsequent to (b), mixing the biological sample with the formulation in the container to yield a mixture comprising the biological sample and the formulation. In some aspects, in step (a), the container comprises a mixture comprising the biological sample and the formulation. In some aspects, the method further comprises, prior to (b), mixing the biological sample with the formulation to yield the mixture and providing the mixture in the container. In some aspects, the container comprises the formulation, and wherein (a) comprises depositing the biological sample in the container comprising the formulation. In some aspects, the container comprises the biological sample, and wherein (a) comprises directing the formulation to the container comprising the biological sample. In some aspects, the at least one microorganism of the biological sample is capable of growth on a culture medium after storage in the container for the time period. In some aspects, the biological sample is a stool sample. In some aspects, the biological sample is obtained from a mammal. In some aspects, the mammal is a human. In some aspects, the formulation has a volume from about 5 milliliters (mL) to about 15 mL. In some aspects, the formulation has a volume of at least about 9 mL. In some aspects, a dilution ratio of the biological sample to the formulation in the container is from about 1:20 to about 1:5. In some aspects, the dilution ratio of the biological sample to the formulation in the container is at least about 1:10. In some aspects, a combined volume of at least the formulation and the biological sample is at least about 80% of a volume of the container when the container is sealed. In some aspects, the antioxidant comprises ascorbic acid, glutathione, dithiothreitol, glutathione peroxidase, a peroxidase, superoxide dismutase, catalase, a derivative thereof, or any combination thereof. In some aspects, the oxygen scavenger comprises thioglycolate, cysteine, glutathione, glutathione peroxidase, peroxidase, catalase, superoxide dismutase, or any combination thereof. In some aspects, the formulation further comprises a cryoprotectant. In some aspects, the cryoprotectant comprises glycerol. In some aspects, the formulation further comprises an electrolyte. In some aspects, the electrolyte comprises sodium chloride, potassium chloride, sodium phosphate, magnesium sulfate, or any combination thereof. In some aspects, the formulation further comprises a metabolite scavenger. In some aspects, the metabolite scavenger comprises activated carbon, glutathione, ascorbic acid, or a combination thereof. In some aspects, the formulation comprises at least two oxygen scavengers. In some aspects, the antioxidant is present in the formulation in an amount ranging from about 10⁻¹⁰ moles per liter (mol/L) to about 10 mol/L. In some aspects, the oxygen scavenger is present in the formulation in an amount ranging from about 10⁻¹⁰ mol/L to about 10 mol/L. In some aspects, the cryoprotectant is present in the formulation in an amount ranging from about 10⁻¹⁰ mol/L to about 10 mol/L. In some aspects, the electrolyte is present in the formulation in an amount ranging from about 10⁻¹⁰ mol/L to about 10 mol/L. In some aspects, the metabolite scavenger is present in the formulation in an amount ranging from about 10⁻¹⁰ mol/L to about 10 mol/L. In some aspects, the mixing comprises shaking or vortexing the container comprising the biological sample and the formulation. In some aspects, the method further comprises, subsequent to (b), depositing the container with a processing unit that processes the biological sample. In some aspects, the depositing comprises providing the container to a parcel delivery service that delivers the container to the processing unit. In some aspects, the method further comprises, prior to (a), receiving a kit comprising one or more of the formulation, the container and a collection device. In some aspects, the kit comprises two or more of the formulation, the container and the collection device. In some aspects, the kit comprises the formulation, the container and the collection device. In some aspects, at least two microorganisms of the biological sample are capable of growth on a culture medium after storage of the biological sample for the time period, which at least two microorganisms comprise the at least one microorganism. In some aspects, at least five microorganisms of the biological sample are capable of growth on the culture medium after storage of the biological sample for the time period, which at least five microorganisms comprise the at least one microorganism. In some aspects, at least ten microorganisms of the biological sample are capable of growth on the culture medium after storage of the biological sample for the time period, which at least ten microorganisms comprise the at least one microorganism. In some aspects, at least 70% of the at least one microorganism of the biological sample is capable of growth on a culture medium after storage of the biological sample for the time period, which at least one microorganism has a relative abundance of at least about 0.001% in the biological sample. In some aspects, at least 80% of the at least one microorganism of the biological sample is capable of growth on the culture medium after storage of the biological sample for the time period, which at least one microorganism has a relative abundance of at least about 0.001% in the biological sample. In some aspects, at least 90% of the at least one microorganism of the biological sample is capable of growth on the culture medium after storage of the biological sample for the time period, which at least one microorganism has a relative abundance of at least about 0.001% in the biological sample. In some aspects, at least 93% of the at least one microorganisms of the biological sample is capable of growth on the culture medium after storage of the biological sample for the time period, which at least one microorganism has a relative abundance of at least about 0.001% in biological sample. In some aspects, at least 80% of the at least one microorganism of the biological sample is capable of growth on a culture medium after storage of the biological sample for the time period, which at least one microorganism has a relative abundance from about 0.001% to about 0.01% in the biological sample. In some aspects, at least 90% of the at least one microorganism of the biological sample is capable of growth on the culture medium after storage of the biological sample for the time period, which at least one microorganism has a relative abundance from about 0.001% to about 0.01% in the biological sample. In some aspects, at least 80% of the at least one microorganisms of the biological sample is capable of growth on a culture medium after storage of the biological sample for the time period, which at least one microorganism has a relative abundance from about 0.01% to about 0.1% in the biological sample. In some aspects, at least 90% of the at least one microorganisms of the biological sample is capable of growth on the culture medium after storage of the biological sample for the time period, which at least one microorganism has a relative abundance of from about 0.01% to about 0.1% in the biological sample. In some aspects, the at least one microorganism of the biological sample is capable of growth on a culture medium after storage of the biological sample for the time period, which at least one microorganism has a relative abundance of less than about 0.001% in the biological sample. In some aspects, the relative abundance of the at least one microorganism is determined by a process comprising sequencing DNA extracted from the at least one microorganism. In some aspects, the culture medium is brain heart infusion (BHI) media, chocolate agar (CHOC) media, Brucella blood agar (BRU) media, or yeast castione fatty acid agar with carbohydrates and blood (YCFAC+B) media. In some aspects, the at least one microorganism comprises at least one anaerobic microorganism. In some aspects, the at least one anaerobic microorganism is an obligate anaerobic microorganism or a facultative anaerobic microorganism. In some aspects, the at least one microorganism belongs to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp.

In various aspects, provided herein is a method of culturing at least one microorganism from a biological sample, comprising: (a) receiving a container comprising (i) the biological sample and (ii) a formulation comprising an antioxidant and an oxygen scavenger; (b) inoculating the stored biological sample or a portion of the biological sample on a culture medium; and (c) incubating the culture medium in an anaerobic chamber for a period of at least six hours such that the at least one microorganism of the stored biological sample grows on the culture medium, wherein, prior to (a), the biological sample is stored in the container comprising the formulation for a time period of least 2 days. In some aspects, a temperature of the anaerobic chamber is between about 20° C. and about 42° C. In some aspects, the method further comprises, prior to (a), the biological sample is stored in the container comprising the formulation for a time period of least 3 days. In some aspects, prior to (a), the biological sample is stored in the container comprising the formulation for a time period of least 4 days. In some aspects, prior to (a), the biological sample is stored in the container comprising the formulation for a time period of least 5 days. In some aspects, the biological sample is a stool sample. In some aspects, the biological sample is obtained from a mammal. In some aspects, the mammal is a human. In some aspects, the formulation has a volume from about 5 milliliter (mL) to about 15 mL. In some aspects, the formulation has a volume of at least about 9 mL. In some aspects, a dilution ratio of the biological sample to the formulation in the container is from about 1:20 to about 1:5. In some aspects, the dilution ratio of the biological sample to the formulation in the container is at least about 1:10. In some aspects, a combined volume of at least the formulation and the biological sample is at least about 80% of a volume of the container when the container is sealed. In some aspects, the antioxidant comprises ascorbic acid, glutathione, dithiothreitol, glutathione peroxidase, a peroxidase, superoxide dismutase, catalase, a derivative thereof, or any combination thereof. In some aspects, the oxygen scavenger comprises thioglycolate, cysteine, glutathione, glutathione peroxidase, peroxidase, catalase, superoxide dismutase, or any combination thereof. In some aspects, the formulation further comprises a cryoprotectant. In some aspects, the cryoprotectant comprises glycerol. In some aspects, the formulation further comprises an electrolyte. In some aspects, the electrolyte comprises sodium chloride, potassium chloride, sodium phosphate, magnesium sulfate, or any combination thereof. In some aspects, the formulation further comprises a metabolite scavenger. In some aspects, the metabolite scavenger comprises activated carbon, glutathione, ascorbic acid, or a combination thereof. In some aspects, the formulation comprises at least two oxygen scavengers. In some aspects, the antioxidant is present in the formulation in an amount ranging from about 10⁻¹⁰ moles per liter (mol/L) to about 10 mol/L. In some aspects, the oxygen scavenger is present in the formulation in an amount ranging from about 10⁻¹⁰ mol/L to about 10 mol/L. In some aspects, the cryoprotectant is present in the formulation in an amount ranging from about 10⁻¹⁰ mol/L to about 10 mol/L. In some aspects, the electrolyte is present in the formulation in an amount ranging from about 10⁻¹⁰ mol/L to about 10 mol/L. In some aspects, the metabolite scavenger is present in the formulation in an amount ranging from about 10⁰ mol/L to about 10 mol/L. In some aspects, at least two microorganisms of the biological sample are capable of growth on the culture medium after storage of the biological sample for the time period, which at least two microorganisms comprise the at least one microorganism. In some aspects, at least five microorganisms of the biological sample are capable of growth on the culture medium after storage of the biological sample for the time period, which at least five microorganisms comprise the at least one microorganism. In some aspects, at least ten microorganisms of the biological sample are capable of growth on the culture medium after storage of the biological sample for the time period, which at least ten microorganisms comprise the at least one microorganism. In some aspects, at least 70% of the at least one microorganism of the biological sample is capable of growth on the culture medium after storage of the biological sample for the time period, which at least one microorganism has a relative abundance of at least about 0.001% in the biological sample. In some aspects, at least 80% of the at least one microorganism of the biological sample is capable of growth on the culture medium after storage of the biological sample for the time period, which at least one microorganism has a relative abundance of at least about 0.001% in the biological sample. In some aspects, at least 90% of the at least one microorganism of the biological sample is capable of growth on the culture medium after storage of the biological sample for the time period, which at least one microorganism has a relative abundance of at least about 0.001% in the biological sample. In some aspects, at least 93% of the at least one microorganisms of the biological sample is capable of growth on the culture medium after storage of the biological sample for the time period, which at least one microorganism has a relative abundance of at least about 0.001% in biological sample. In some aspects, at least 80% of the at least one microorganism of the biological sample is capable of growth on the culture medium after storage of the biological sample for the time period, which at least one microorganism has a relative abundance from about 0.001% to about 0.01% in the biological sample. In some aspects, at least 90% of the at least one microorganism of the biological sample is capable of growth on the culture medium after storage of the biological sample for the time period, which at least one microorganism has a relative abundance from about 0.001% to about 0.01% in the biological sample. In some aspects, at least 80% of the at least one microorganisms of the biological sample is capable of growth on the culture medium after storage of the biological sample for the time period, which at least one microorganism has a relative abundance from about 0.01% to about 0.1% in the biological sample. In some aspects, at least 90% of the at least one microorganisms of the biological sample is capable of growth on the culture medium after storage of the biological sample for the time period, which at least one microorganism has a relative abundance of from about 0.01% to about 0.1% in the biological sample. In some aspects, the at least one microorganism of the biological sample is capable of growth on the culture medium after storage of the biological sample for the time period, which at least one microorganism has a relative abundance of less than about 0.001% in the biological sample. In some aspects, the relative abundance of the at least one microorganism is determined by a process comprising sequencing DNA extracted from the at least one microorganisms. In some aspects, the culture medium is a BHI, a CHOC or a YCFAC+B medium. In some aspects, the biological sample is stored in the container comprising the biological sample and the formulation for the time period at a temperature of about 20° C. to about 40° C. In some aspects, the biological sample is stored in the container comprising the biological sample and the formulation for the time period at the temperature followed by a freeze-thawing process. In some aspects, the freeze-thawing process comprises storing the biological sample in the container comprising the biological sample and the formulation for a time period of 1 day to about 100 days at a temperature of less than about −70° C. In some aspects, the freeze-thawing process decreases a percentage of the at least one microorganism that grows on the culture medium by less than about 20% compared to a growth of the at least one microorganism that is subjected to the freeze-thawing process. In some aspects, the at least one microorganism comprises at least one anaerobic microorganism. In some aspects, the at least one anaerobic microorganism is an obligate anaerobic microorganism or a facultative anaerobic microorganism. In some aspects, the at least one microorganism belongs to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp.

In various aspects, provided herein is a system for obtaining a biological sample comprising at least one microorganism from a subject and preserving a viability of the at least one microorganism, comprising: (i) a formulation comprising an antioxidant and at least one oxygen scavenger; (ii) a container configured to (a) store the biological sample and the formulation, and (b) to be sealed; and (iii) a collection device configured to obtain the biological sample from the subject, wherein the formulation is configured to preserve the viability of the at least one microorganism for a time period of at least 2 days, such that at least 50% of the at least one microorganism is capable of growth on a culture medium after the time period. In some aspects, the formulation further comprises an electrolyte. In some aspects, the electrolyte comprises sodium chloride, potassium chloride, sodium phosphate, magnesium sulfate, or any combination thereof. In some aspects, the antioxidant is a peroxidase, an ascorbic acid, a glutathione, a dithiothreitol, a derivative thereof, or any combination thereof. In some aspects, the at least one oxygen scavenger comprises sodium thioglycolate, a cysteine, a peroxidase, a catalase, a dismutase, or any combination thereof. In some aspects, the at least one oxygen scavenger comprise at least two oxygen scavengers. In some aspects, the formulation further comprises a metabolite scavenger. In some aspects, the metabolite scavenger comprises activated carbon. In some aspects, the formulation further comprises a cryoprotectant. In some aspects, the cryoprotectant comprises glycerol. In some aspects, the time period is at least 3 days. In some aspects, the time period is at least 4 days. In some aspects, the time period is at least 5 days. In some aspects, the antioxidant is present in the formulation in an amount ranging from about 10⁻¹⁰ moles per liter (mol/L) to about 10 mol/L. In some aspects, the at least one oxygen scavenger is present in the formulation in an amount ranging from about 10⁻¹⁰ mol/L to about 10 mol/L. In some aspects, the cryoprotectant is present in the formulation in an amount ranging from about 10⁻¹⁰ mol/L to about 10 mol/L. In some aspects, the electrolyte is present in the formulation in an amount ranging from about 10⁻¹⁰ mol/L to about 10 mol/L. In some aspects, the metabolite scavenger is present in the formulation in an amount ranging from about 10⁻¹⁰ mol/L to about 10 mol/L. In some aspects, the formulation has a volume from about 5 milliliter (mL) to about 15 mL. In some aspects, the formulation has a volume of at least about 9 mL. In some aspects, a dilution ratio of the biological sample to the formulation in the container is from about 1:20 to about 1:5. In some aspects, the dilution ratio of the biological sample to the formulation in the container is at least about 1:10. In some aspects, the collection device is a scoop, a spatula, or a brush. In some aspects, the at least one microorganism comprises at least one anaerobic microorganism. In some aspects, the at least one microorganism belongs to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp. In some aspects, the biological sample comprises at least two microorganisms, which at least two microorganisms comprise the at least one microorganisms. In some aspects, the biological sample comprises at least five microorganisms, which at least five microorganisms comprise the at least one microorganisms. In some aspects, the biological sample comprises at least ten microorganisms, which at least ten microorganisms comprise the at least one microorganisms. In some aspects, the biological sample is a stool sample. In some aspects, the subject is a mammal. In some aspects, the mammal is a human.

Further provided herein is a kit for storing a biological sample comprising at least one microorganism from a subject, comprising: a formulation comprising an antioxidant and an oxygen scavenger; a container configured to store the biological sample and the formulation, which container is configured to be sealed; and instructions that direct a user to seal the container comprising the formulation and the biological sample, to thereby preserve a viability of the at least one microorganism for a time period of at least 2 days. In some aspects, the instructions further direct the user to place the formulation and the biological sample in the container prior to the sealing of the container. In some aspects, the formulation is a liquid, and wherein the container comprises the formulation and a head space filled with a gas, wherein the gas comprises less than 1% oxygen. In some aspects, the antioxidant comprises ascorbic acid, glutathione, dithiothreitol, glutathione peroxidase, a peroxidase, superoxide dismutase, catalase, a derivative thereof, or any combination thereof. In some aspects, the antioxidant comprises L-ascorbic acid and dithiothreitol. In some aspects, the oxygen scavenger comprises thioglycolate, cysteine, glutathione, glutathione peroxidase, peroxidase, catalase, superoxide dismutase, or any combination thereof. In some aspects, the oxygen scavenger comprises sodium thioglycolate, L-cysteine, glutathione, glutathione peroxidase, peroxidase, catalase and superoxide dismutase. In some aspects, the antioxidant and the oxygen scavenger comprise different compounds. In some aspects, the formulation further comprises a cryoprotectant. In some aspects, the cryoprotectant comprises glycerol. In some aspects, the kit can further comprise a collection device configured to obtain the biological sample from the subject. In some aspects, the collection device is a scoop, a spatula, or a brush. In some aspects, the formulation further comprises an electrolyte. In some aspects, the electrolyte comprises sodium chloride, potassium chloride, sodium phosphate, potassium phosphate, magnesium sulfate, or any combination thereof. In some aspects, the electrolyte comprises sodium phosphate, sodium chloride, potassium chloride, potassium phosphate and magnesium sulfate. In some aspects, the formulation further comprises a metabolite scavenger. In some aspects, the metabolite scavenger comprises activated carbon, glutathione, ascorbic acid, or a combination thereof. In some aspects, the antioxidant, the oxygen scavenger, and the metabolite scavenger comprise different compounds. In some aspects, the formulation comprises at least two oxygen scavengers comprising the oxygen scavenger. In some aspects, the antioxidant is present in the formulation in an amount ranging from about 10⁻² gram (g) per liter (g/L) to about 10 g/L. In some aspects, the antioxidant is present in the formulation in an amount of about 0.2 g/L. In some aspects, the oxygen scavenger is present in the formulation in an amount ranging from about 10⁻² g/L to about 10² g/L. In some aspects, the oxygen scavenger is present in the formulation in an amount ranging from about 0.1 g/L to about 1 g/L. In some aspects, the oxygen scavenger is an enzyme, and wherein the enzyme is present in the formulation in an amount ranging from about 5 units (U) to about 10⁶ U. In some aspects, the enzyme is present in the formulation in an amount ranging from about 20 U to about 140,000 U. In some aspects, the cryoprotectant is present in the formulation in an amount ranging from about 5 g/L to about 500 g/L. In some aspects, the cryoprotectant is present in the formulation in an amount ranging from about 50 g/L to about 300 g/L. In some aspects, the electrolyte is present in the formulation in an amount ranging from about 10⁻² g/L to about 10 g/L. In some aspects, the electrolyte is present in the formulation in an amount ranging from about 10⁻¹ g/L to about 10 g/L. In some aspects, the metabolite scavenger is present in the formulation in an amount ranging from about 10⁻² g/L to about 10² g/L. In some aspects, the metabolite scavenger is present in the formulation in an amount of about 0.2 g/L. In some aspects, the formulation comprises the components listed in any one of TABLES 1-4. In some aspects, the formulation consists of the components listed in any one of TABLES 1-4. In some aspects, the formulation comprises the components listed in TABLE 4. In some aspects, the formulation consists of the components listed in TABLE 4. In some aspects, the instructions direct the user to use the collection device to obtain the biological sample from the subject. In some aspects, the instructions direct the user to subject the container comprising the formulation and the biological sample to conditions sufficient to generate a homogeneous mixture comprising the formulation and the biological sample. In some aspects, the formulation has a volume from about 5 milliliters (mL) to about 15 mL. In some aspects, the formulation has a volume of at least about 9 mL. In some aspects, the biological sample comprises at least 10 different species of microorganisms. In some aspects, the formulation is configured to preserve the viability of at least 10, at least 20, at least 50, or at least 100 different species of microorganisms. In some aspects, the formulation is configured to preserve the viability of at least 10 different species of microorganisms. In some aspects, the formulation is configured to preserve the viability of at least 20 different species of microorganisms. In some aspects, the formulation is configured to preserve the viability of at least 50 different species of microorganisms. In some aspects, the formulation is configured to preserve the viability of at least 100 different species of microorganisms. In some aspects, the formulation is configured to preserve the viability of at least 150 different species of microorganisms. In some aspects, the at least 10 different species of microorganisms comprise at least 10 abundant species present at a relative abundance of >0.1% in the biological sample, and the formulation is configured to preserve the viability of the at least 10 abundant species. In some aspects, the at least one abundant species comprises at least 10 abundant species and the formulation is configured to preserve the viability of the at least 10 abundant species. In some aspects, the at least 10 different species of microorganisms comprise at least 10 low abundance species present at a relative abundance of between 0.01% and 0.1% in the biological sample, and the formulation is configured to preserve the viability of the at least 10 low abundance species. In some aspects, the at least one low abundance species comprises at least 10 abundant species and the formulation is configured to preserve the viability of the at least 10 low abundance species. In some aspects, the at least 10 different species of microorganisms comprise at least 10 rare species present at a relative abundance of between 0.001% and 0.01% in the biological sample, and the formulation is configured to preserve the viability of the at least 10 rare species. In some aspects, the at least one abundant species comprises at least 10 rare species and the formulation is configured to preserve the viability of the at least 10 rare species. In some aspects, the relative abundance is determined by a process comprising culturing the at least 10 different species of microorganisms from the formulation on a nutrient agar and sequencing a plurality of DNA polynucleotides extracted from the at least 10 different species of microorganisms. In some aspects, the at least one microorganism comprises at least one anaerobic microorganism. In some aspects, the at least one anaerobic microorganism comprises an obligate anaerobic microorganism or a facultative anaerobic microorganism. In some aspects, the at least one microorganism comprises a microorganism belonging to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp. In some aspects, the at least one microorganism comprises a microorganism belonging to a species selected from the group consisting of Faecalibacterium prausnitzii, Prevotella copri, Blautia obeum, Eubacterium rectale, Coprococcus eutactus, Fusicatenibacter saccharivorans, Roseburia faecis, Eubacterium hallii, Bifidobacterium adolescentis, Collinsella aerofaciens, Ali stipes putredinis, Akkermansia muciniphila, Bifidobacterium bifidum, Coprococcus comes, Dorea longicatena, Ruminiclostridium Eubacterium siraeum, Roseburia inulinivorans, Bacteroides vulgatus, Bifidobacterium pseudocatenulatum, Parabacteroides merdae, Blautia Ruminococcus gnavus, Enterorhabdus caecimuris, Anaerostipes hadrus, Ali stipes finegoldii, and Eubacterium ramulus. In some aspects, the at least one microorganism comprises a microorganism belonging to a species selected from the group consisting of Faecalibacterium prausnitzii, Akkermansia muciniphila, Prevotella copri, and Bifidobacterium bifidum. In some aspects, the biological sample is a stool sample. In some aspects, a shelf-life of the kit is at least 1, 2, 3, 4, or 5 months.

Provided herein is a method for forming a container for preserving at least one microorganism, comprising: (a) providing the container comprising a formulation comprising an antioxidant and an oxygen scavenger; and (b) sealing the container; wherein (a) and (b) are performed under anaerobic conditions, wherein the container comprising the formulation is configured to preserve the at least one microorganism for a time period of at least 2 days. In some aspects, the anaerobic conditions comprise an oxygen gas content of less than 1%. In some aspects, a shelf life of the formulation within the container is at least 1, 2, 3, 4, or 5 months. In some aspects, a shelf life of the formulation within the container is at least three months.

Provided herein is a method for preserving a biological sample comprising at least one microorganism, the method comprising: (a) providing a container comprising (i) the biological sample and (ii) a formulation comprising an antioxidant and an oxygen scavenger; and (b) sealing the container, to thereby preserve a viability of the at least one microorganism for a time period of at least 2 days. In some aspects, the method comprises preserving the viability of the at least one microorganism for a time period of at least 3 days, at least 4 days, or at least 5 days. In some aspects, the method comprises preserving the viability of the at least one microorganism for a time period of at least 4 days. In some aspects, the method comprises preserving the viability of the at least one microorganism for a time period of at least 5 days. In some aspects, in step (a), the container comprises a mixture comprising the biological sample and the formulation. In some aspects, the method further comprises, subsequent to (b), mixing the biological sample with the formulation in the container to yield a mixture comprising the biological sample and the formulation. In some aspects, the method further comprises, prior to (b), mixing the biological sample with the formulation to yield the mixture and providing the mixture in the container. In some aspects, the container comprises the formulation, and wherein (a) comprises placing the biological sample in the container comprising the formulation. In some aspects, the container comprises the biological sample, and wherein (a) comprises placing the formulation in the container comprising the biological sample. In some aspects, the at least one microorganism of the biological sample is capable of growth on a culture medium after storage in the container for the time period. In some aspects, the biological sample is a stool sample. In some aspects, the biological sample is obtained from a mammal. In some aspects, the mammal is a human. In some aspects, the formulation has a volume from about 5 milliliters (mL) to about 15 mL. In some aspects, the formulation has a volume of at least about 9 mL. In some aspects, a dilution ratio of the biological sample to the formulation in the container is from about 1:20 to about 1:5. In some aspects, the dilution ratio of the biological sample to the formulation in the container is at least about 1:10. In some aspects, a combined volume of at least the formulation and the biological sample is at least about 80% of a volume of the container when the container is sealed. In some aspects, the antioxidant comprises ascorbic acid, glutathione, dithiothreitol, glutathione peroxidase, a peroxidase, superoxide dismutase, catalase, a derivative thereof, or any combination thereof. In some aspects, the antioxidant comprises L-ascorbic acid and dithiothreitol. In some aspects, the oxygen scavenger comprises thioglycolate, cysteine, glutathione, glutathione peroxidase, peroxidase, catalase, superoxide dismutase, or any combination thereof. In some aspects, the oxygen scavenger comprises sodium thioglycolate, L-cysteine, glutathione, glutathione peroxidase, peroxidase, catalase and superoxide dismutase. In some aspects, the antioxidant and the oxygen scavenger comprise different compounds. In some aspects, the formulation further comprises a cryoprotectant. In some aspects, the cryoprotectant comprises glycerol. In some aspects, the formulation further comprises an electrolyte. In some aspects, the electrolyte comprises sodium chloride, potassium chloride, sodium phosphate, potassium phosphate, magnesium sulfate, or any combination thereof. In some aspects, the electrolyte comprises sodium phosphate, sodium chloride, potassium chloride, potassium phosphate and magnesium sulfate. In some aspects, the formulation further comprises a metabolite scavenger. In some aspects, the metabolite scavenger comprises activated carbon, glutathione, ascorbic acid, or a combination thereof. In some aspects, the antioxidant, the oxygen scavenger, and the metabolite scavenger comprise different compounds. In some aspects, the formulation comprises at least two oxygen scavengers. In some aspects, the antioxidant is present in the formulation in an amount ranging from about 10⁻³ g/L to about 10² g/L. In some aspects, the antioxidant is present in the formulation in an amount of about 0.2 g/L. In some aspects, the oxygen scavenger is present in the formulation in an amount ranging from about 10⁻³ g/L to about 10 g/L. In some aspects, the oxygen scavenger is present in the formulation in an amount ranging from about 0.1 g/L to about 1 g/L. In some aspects, the oxygen scavenger comprises an enzyme, and wherein the enzyme is present in the formulation in an amount ranging from about 5 units (U) to about 10⁶ U. In some aspects, the enzyme is present in the formulation in an amount ranging from about 20 U to about 140,000 U. In some aspects, the cryoprotectant is present in the formulation in an amount ranging from about 5 g/L to about 500 g/L. In some aspects, the cryoprotectant is present in the formulation in an amount ranging from about 50 g/L to about 300 g/L. In some aspects, the electrolyte is present in the formulation in an amount ranging from about 10⁻² g/L to about 10 g/L. In some aspects, the electrolyte is present in the formulation in an amount ranging from about 10⁻¹ g/L to about 10 g/L. In some aspects, the formulation comprises the components listed in any one of TABLES 1-4. In some aspects, the formulation consists of the components listed in any one of TABLES 1-4. In some aspects, the formulation comprises the components listed in TABLE 4. In some aspects, the formulation consists of the components listed in TABLE 4. In some aspects, the metabolite scavenger is present in the formulation in an amount ranging from about 10⁻² g/L to about 10² g/L. In some aspects, the mixing comprises shaking or vortexing the container comprising the biological sample and the formulation. In some aspects, the method further comprises, subsequent to (b), depositing the container with a processing unit that processes the biological sample. In some aspects, the depositing comprises providing the container to a parcel delivery service that delivers the container to the processing unit. In some aspects, the method further comprises, prior to (a), receiving a kit comprising one or more of the formulation, the container and a collection device. In some aspects, the kit comprises the formulation, the container and the collection device. In some aspects, the kit comprises two or more of the formulation, the container and the collection device. In some aspects, the at least one microorganism of the biological sample is capable of growth on a culture medium at a relative abundance of at least 0.001% after storage of the biological sample for the time period. In some aspects, the culture medium comprises a brain heart infusion broth (BHI) medium, a chocolate agar (CHOC) medium, a brucella blood agar (BRU) medium, or a yeast casitone fatty acids agar with carbohydrates and sheep blood (YCFAC+B) medium. In some aspects, the relative abundance of the at least one microorganism on the culture medium is determined by a process comprising sequencing DNA extracted from the at least one microorganism. In some aspects, the biological sample comprising at least one microorganism comprises at least 10 different species of microorganisms. In some aspects, the viability of the at least 10 different species of microorganisms is preserved. In some aspects, the at least 10 different species of microorganisms comprises at least 20 different species of microorganisms and the viability of the at least 20 different species of microorganisms is preserved. In some aspects, the at least 10 different species of microorganisms comprises at least 50 different species of microorganisms and the viability of the at least 50 different species of microorganisms is preserved. In some aspects, the at least 10 different species of microorganisms comprises at least 100 different species of microorganisms and the viability of the at least 100 different species of microorganisms is preserved. In some aspects, a relative abundance of each species of the at least 10 different species of microorganisms in the biological sample is at least 0.001% or at least 0.01%. In some aspects, the relative abundance of each species of the at least 10 different species of microorganisms in the biological sample is at least 0.01%. In some aspects, the at least 10 different species of microorganisms comprises at least one abundant species present at a relative abundance of >0.1% and the viability of the at least one abundant species is preserved. In some aspects, the at least 10 different species of microorganisms comprises at least 10 abundant species present at a relative abundance of >0.1% in the biological sample and the viability of the at least 10 abundant species is preserved. In some aspects, the at least 10 different species of microorganisms comprises at least 30 abundant species present at a relative abundance of >0.1% in the biological sample and the viability of the at least 30 abundant species is preserved. In some aspects, the at least 10 different species of microorganisms comprises at least one low abundance species present at a relative abundance of between 0.01% and 0.1% in the biological sample and the viability of the at least one low abundance species is preserved. In some aspects, the at least 10 different species of microorganisms comprises at least 10 low abundance species present at a relative abundance of between 0.01% and 0.1% in the biological sample and the viability of the at least 10 low abundance species is preserved. In some aspects, the at least 10 different species of microorganisms comprises at least 30 low abundance species present at a relative abundance of between 0.01% and 0.1% in the biological sample and the viability of the at least 30 low abundance species is preserved. In some aspects, the at least 10 different species of microorganisms comprises at least one rare species present at a relative abundance of between 0.001% and 0.01% in the biological sample and the viability of the at least one rare species is preserved. In some aspects, the at least 10 different species of microorganisms comprises at least 10 rare species present at a relative abundance of between 0.001% and 0.01% in the biological sample and the viability of the at least 10 rare species is preserved. In some aspects, the at least 10 different species of microorganisms comprises at least 30 rare species present at a relative abundance of between 0.001% and 0.01% in the biological sample and the viability of the at least 30 rare species is preserved. In some aspects, the relative abundance of each of at least 80% or at least 90% of the at least 10 different species of microorganisms on the culture media is at least 10% of a relative abundance of the species in the biological sample. In some aspects, the relative abundance of each of at least 90% of the at least 10 different species of microorganisms on the culture media is at least 1% of a relative abundance of the species in the biological sample. In some aspects, the at least one microorganism comprises at least one anaerobic microorganism. In some aspects, the at least one anaerobic microorganism is an obligate anaerobic microorganism or a facultative anaerobic microorganism. In some aspects, the at least one microorganism comprises a microorganism belonging to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp. In some aspects, the at least one microorganism comprises a microorganism belonging to a species selected from the group consisting of Faecalibacterium prausnitzii, Prevotella copri, Blautia obeum, Eubacterium rectale, Coprococcus eutactus, Fusicatenibacter saccharivorans, Roseburia faecis, Eubacterium hallii, Bifidobacterium adolescentis, Collinsella aerofaciens, Alistipes putredinis, Akkermansia muciniphila, Bifidobacterium bifidum, Coprococcus comes, Dorea longicatena, Ruminiclostridium Eubacterium siraeum, Roseburia inulinivorans, Bacteroides vulgatus, Bifidobacterium pseudocatenulatum, Parabacteroides merdae, Blautia Ruminococcus gnavus, Enterorhabdus caecimuris, Anaerostipes hadrus, Alistipes finegoldii, and Eubacterium ramulus. In some aspects, the at least one microorganism comprises a microorganism belonging to a species selected from the group consisting of Faecalibacterium prausnitzii, Akkermansia muciniphila, Prevotella copri, and Bifidobacterium bifidum.

Further provided herein, in various aspects, is a method of culturing at least one microorganism from a biological sample, comprising: (a) receiving a container comprising (i) the biological sample and (ii) a formulation comprising an antioxidant and an oxygen scavenger; (b) inoculating the biological sample or a portion of the biological sample on a culture medium; and (c) incubating the culture medium in an anaerobic chamber for a period of at least six hours such that the at least one microorganism of the biological sample grows on the culture medium, wherein, prior to (a), the biological sample is stored in the container comprising the formulation for a time period of least 2 days. In some aspects, a temperature in the anaerobic chamber is between about 20° C. and about 42° C. In some aspects, the method can further comprise, prior to (a), storing the biological sample in the container comprising the formulation for a time period of least 3 days, at least 4 days, or at least 5 days. In some aspects, the method can further comprise, prior to (a), storing the biological sample in the container comprising the formulation for a time period of least 4 days. In some aspects, the method can further comprise, prior to (a), storing the biological sample in the container comprising the formulation for a time period of least 5 days. In some aspects, the biological sample is a stool sample. In some aspects, the biological sample is obtained from a mammal. In some aspects, the mammal is a human. In some aspects, the formulation has a volume from about 5 milliliter (mL) to about 15 mL. In some aspects, the formulation has a volume of at least about 9 mL. In some aspects, a dilution ratio of the biological sample to the formulation in the container is from about 1:20 to about 1:5. In some aspects, the dilution ratio of the biological sample to the formulation in the container is at least about 1:10. In some aspects, a combined volume of at least the formulation and the biological sample is at least about 80% of a volume of the container when the container is sealed. In some aspects, the antioxidant comprises ascorbic acid, glutathione, dithiothreitol, glutathione peroxidase, a peroxidase, superoxide dismutase, catalase, a derivative thereof, or any combination thereof. In some aspects, the antioxidant comprises L-ascorbic acid and dithiothreitol. In some aspects, the oxygen scavenger comprises thioglycolate, cysteine, glutathione, glutathione peroxidase, peroxidase, catalase, superoxide dismutase, or any combination thereof. In some aspects, the oxygen scavenger comprises sodium thioglycolate, L-cysteine, glutathione, glutathione peroxidase, peroxidase, catalase and superoxide dismutase. In some aspects, the antioxidant and the oxygen scavenger comprise different compounds. In some aspects, the formulation further comprises a cryoprotectant. In some aspects, the cryoprotectant comprises glycerol. In some aspects, the formulation further comprises an electrolyte. In some aspects, the electrolyte comprises sodium chloride, potassium chloride, sodium phosphate, potassium phosphate, magnesium sulfate, or any combination thereof. In some aspects, the electrolyte comprises sodium phosphate, sodium chloride, potassium chloride, potassium phosphate and magnesium sulfate. In some aspects, the formulation further comprises a metabolite scavenger. In some aspects, the metabolite scavenger comprises activated carbon, glutathione, ascorbic acid, or a combination thereof. In some aspects, the antioxidant, the oxygen scavenger, and the metabolite scavenger comprise different compounds. In some aspects, the formulation comprises at least two oxygen scavengers. In some aspects, the antioxidant is present in the formulation in an amount ranging from about 10⁻³ g/L to about 10² g/L. In some aspects, the antioxidant is present in the formulation in an amount of about 0.2 g/L. In some aspects, the oxygen scavenger is present in the formulation in an amount ranging from about 10⁻³ g/L to about 10 g/L. In some aspects, the oxygen scavenger is present in the formulation in an amount ranging from about 0.1 g/L to about 1 g/L. In some aspects, the oxygen scavenger comprises an enzyme, and wherein the enzyme is present in the formulation in an amount ranging from about 5 units (U) to about 10⁶ U. In some aspects, the enzyme is present in the formulation in an amount ranging from about 20 U to about 140,000 U. In some aspects, the cryoprotectant is present in the formulation in an amount ranging from about 5 g/L to about 500 g/L. In some aspects, the cryoprotectant is present in the formulation in an amount ranging from about 50 g/L to about 300 g/L. In some aspects, the electrolyte is present in the formulation in an amount ranging from about 10⁻² g/L to about 10 g/L. In some aspects, the electrolyte is present in the formulation in an amount ranging from about 10⁻¹ g/L to about 10 g/L. In some aspects, the formulation comprises the components listed in any one of TABLES 1-4. In some aspects, the formulation consists of the components listed in any one of TABLES 1-4. In some aspects, the formulation comprises the components listed in TABLE 4. In some aspects, the formulation consists of the components listed in TABLE 4. In some aspects, the metabolite scavenger is present in the formulation in an amount ranging from about 10⁻² g/L to about 10² g/L. In some aspects, at least two microorganisms of the biological sample are capable of growth on the culture medium after storage of the biological sample for the time period, which at least two microorganisms comprise the at least one microorganism. In some aspects, at least five microorganisms of the biological sample are capable of growth on the culture medium after storage of the biological sample for the time period, which at least five microorganisms comprise the at least one microorganism. In some aspects, at least ten microorganisms of the biological sample are capable of growth on the culture medium after storage of the biological sample for the time period, which at least ten microorganisms comprise the at least one microorganism. In some aspects, the biological sample comprises at least 10 different species of viable microorganisms. In some aspects, at least 10, at least 20, or at least 50 of the at least 10 different species of microorganisms of the biological sample grow on the culture medium, which at least 10, at least 20, or at least 50 different species of microorganisms, respectively, have a relative abundance of at least about 0.001% in the biological sample. In some aspects, at least 10 of the at least 10 different species of microorganisms of the biological sample grow on the culture medium, which at least 10 different species of microorganisms, respectively, have a relative abundance of at least about 0.001% in the biological sample. In some aspects, at least 20 of the at least 10 different species of microorganisms of the biological sample grow on the culture medium, which at least 20 different species of microorganisms, respectively, have a relative abundance of at least about 0.001% in the biological sample. In some aspects, at least 10 of the at least 50 different species of microorganisms of the biological sample grow on the culture medium, which at least 50 different species of microorganisms, respectively, have a relative abundance of at least about 0.001% in the biological sample. In some aspects, at least 100 of the at least 10 different species of microorganisms of the biological sample grow on the culture medium, which at least 100 different species of microorganisms, respectively, have a relative abundance of at least about 0.001% in the biological sample. In some aspects, at least 20 of the at least 10 different species of microorganisms of the biological sample grow on the culture medium, which at least 20 different species of microorganisms have a relative abundance of at least about 0.001% in the biological sample. In some aspects, at least 50 of the at least 10 different species of microorganisms of the biological sample grow on the culture medium, which at least 50 different species of microorganisms have a relative abundance of at least about 0.001% in the biological sample. In some aspects, at least 10, at least 20, or at least 50 of the at least 10 different species of microorganisms of the biological sample grow on the culture medium, which at least 10, at least 20, or at least 50 different species of microorganisms, respectively, have a relative abundance of at least about 0.01% in the biological sample. In some aspects, at least 10 of the at least 10 different species of microorganisms of the biological sample grow on the culture medium, which at least 10 species of microorganisms, respectively, have a relative abundance of at least about 0.01% in the biological sample. In some aspects, at least 20 of the at least 10 different species of microorganisms of the biological sample grow on the culture medium, which at least 20 species of microorganisms, respectively, have a relative abundance of at least about 0.01% in the biological sample. In some aspects, at least 50 of the at least 10 different species of microorganisms of the biological sample grow on the culture medium, which at least 50 species of microorganisms, respectively, have a relative abundance of at least about 0.01% in the biological sample. In some aspects, at least 20 of the at least 10 different species of microorganisms of the biological sample grow on the culture medium, which at least 10 different species of microorganisms have a relative abundance of at least about 0.01% in the biological sample. In some aspects, at least 50 of the at least 10 different species of microorganisms of the biological sample grow on the culture medium, which at least 10 different species of microorganisms have a relative abundance of at least about 0.01% in the biological sample. In some aspects, at least 10 or at least 30 of the at least 10 different species of microorganisms of the biological sample grow on the culture medium, which at least 10 or at least 30 different species of microorganisms, respectively, have a relative abundance of 0.001%-0.01% in the biological sample. In some aspects, at least 30 of the at least 10 different species of microorganisms of the biological sample grow on the culture medium, which at least 10 different species of microorganisms have a relative abundance of 0.001%-0.01% in the biological sample. In some aspects, at least 10 or at least 30 of the at least 10 different species of microorganisms of the biological sample grow on the culture medium, which at least 10 or at least 30 different species of microorganisms, respectively, have a relative abundance of 0.01%-0.1% in the biological sample. In some aspects, at least 30 of the at least 10 different species of microorganisms of the biological sample grow on the culture medium, which at least 10 different species of microorganisms have a relative abundance of 0.01%-0.1% in the biological sample. In some aspects, at least 10 or at least 30 of the at least 10 different species of microorganisms of the biological sample grow on the culture medium, which at least 10 or at least 30 different species of microorganisms, respectively, have a relative abundance of at least about 0.1% in the biological sample. In some aspects, at least 30 of the at least 10 different species of microorganisms of the biological sample grow on the culture medium, which at least 30 different species of microorganisms have a relative abundance of at least about 0.1% in the biological sample. In some aspects, a relative abundance of at least 80% or at least 90% of each of the at least 10 different species of microorganisms on the culture medium is at least 10% of a relative abundance of the species of microorganism in the biological sample. In some aspects, a relative abundance of at least 90% of each of the at least 10 different species of microorganisms on the culture medium is at least 10% of a relative abundance of the species of microorganism in the biological sample. In some aspects, a relative abundance of at least 90% of each of the at least 10 different species of microorganisms on the culture medium is at least 1% of a relative abundance of the species of microorganism in the biological sample. In some aspects, the relative abundance of the at least 10 different species of microorganisms is determined by a process comprising sequencing DNA extracted from the at least 10 different species of microorganisms. In some aspects, the culture medium comprises a brain heart infusion broth (BHI) medium, a chocolate agar (CHOC) medium, a brucella blood agar (BRU) medium, or a yeast casitone fatty acids agar with carbohydrates and sheep blood (YCFAC+B) medium. In some aspects, the biological sample is stored in the container comprising the biological sample and the formulation for the time period at a temperature of about 20° C. to about 40° C. In some aspects, the biological sample is stored in the container comprising the biological sample and the formulation for the time period at the temperature followed by a freeze-thawing process. In some aspects, the freeze-thawing process comprises comprising storing the biological sample in the container comprising the biological sample and the formulation for a time period of 1 day to about 100 days at a temperature of less than about −70° C. In some aspects, the freeze-thawing process decreases a percentage of the at least one microorganism that grows on the culture medium by less than about 20% compared to a growth of the at least one microorganism that is not subjected to the freeze-thawing process. In some aspects, the at least one microorganism comprises at least one anaerobic microorganism. In some aspects, the at least one anaerobic microorganism is an obligate anaerobic microorganism or a facultative anaerobic microorganism. In some aspects, the at least one microorganism comprises a microorganism belonging to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp. In some aspects, the at least one microorganism comprises a microorganism belonging to a species selected from the group consisting of Faecalibacterium prausnitzii, Prevotella copri, Blautia obeum, Eubacterium rectale, Coprococcus eutactus, Fusicatenibacter saccharivorans, Roseburia faecis, Eubacterium hallii, Bifidobacterium adolescentis, Collinsella aerofaciens, Alistipes putredinis, Akkermansia muciniphila, Bifidobacterium bifidum, Coprococcus comes, Dorea longicatena, Ruminiclostridium Eubacterium siraeum, Roseburia inulinivorans, Bacteroides vulgatus, Bifidobacterium pseudocatenulatum, Parabacteroides merdae, Blautia Ruminococcus gnavus, Enterorhabdus caecimuris, Anaerostipes hadrus, Alistipes finegoldii, and Eubacterium ramulus. In some aspects, the at least one microorganism comprises a microorganism belonging to a species selected from the group consisting of Faecalibacterium prausnitzii, Akkermansia muciniphila, Prevotella copri, and Bifidobacterium bifidum. In some aspects, the formulation has a shelf-life of at least 2, 3, 4, 5, 6, or more months.

Further provided herein is a system for obtaining a biological sample comprising at least one microorganism from a subject and preserving a viability of the at least one microorganism, comprising: (i) a formulation comprising an antioxidant and at least one oxygen scavenger; (ii) a container configured to (a) store the biological sample and the formulation, and (b) to be sealed; and (iii) a collection device configured to obtain the biological sample from the subject, wherein the formulation is configured to preserve the viability of the at least one microorganism for a time period of at least 2 days, such that the at least one microorganism is capable of growth on a culture medium after the time period. In some aspects, the formulation further comprises an electrolyte. In some aspects, the electrolyte comprises sodium chloride, potassium chloride, sodium phosphate, potassium sulfate, magnesium sulfate, or any combination thereof. In some aspects, the electrolyte comprises sodium phosphate, sodium chloride, potassium chloride, potassium phosphate and magnesium sulfate. In some aspects, the antioxidant is a peroxidase, an ascorbic acid, a glutathione, a dithiothreitol, a derivative thereof, or any combination thereof. In some aspects, the antioxidant comprises L-ascorbic acid and dithiothreitol. In some aspects, the at least one oxygen scavenger comprises sodium thioglycolate, a cysteine, a peroxidase, a catalase, a dismutase, or any combination thereof. In some aspects, the oxygen scavenger comprises sodium thioglycolate, L-cysteine, glutathione, glutathione peroxidase, peroxidase, catalase and superoxide dismutase. In some aspects, the at least one oxygen scavenger comprise at least two oxygen scavengers. In some aspects, the formulation further comprises a metabolite scavenger. In some aspects, the metabolite scavenger comprises activated carbon. In some aspects, the formulation further comprises a cryoprotectant. In some aspects, the cryoprotectant comprises glycerol. In some aspects, the time period is at least 3 days. In some aspects, the time period is at least 4 days. In some aspects, the time period is at least 5 days. In some aspects, the antioxidant is present in the formulation in an amount ranging from about 10⁻³ g/L to about 10 g/L. In some aspects, the antioxidant is present in the formulation in an amount of about 0.2 g/L. In some aspects, the at least one oxygen scavenger is present in the formulation in an amount ranging from about 10⁻³ g/L to about 10 g/L. In some aspects, the oxygen scavenger is present in the formulation in an amount ranging from about 0.1 g/L to about 1 g/L. In some aspects, the oxygen scavenger comprises an enzyme, and wherein the enzyme is present in the formulation in an amount ranging from about 5 units (U) to about 10⁶ U. In some aspects, the enzyme is present in the formulation in an amount ranging from about 20 U to about 140,000 U. In some aspects, the cryoprotectant is present in the formulation in an amount ranging from about 5 g/L to about 500 g/L. In some aspects, the cryoprotectant is present in the formulation in an amount ranging from about 50 g/L to about 300 g/L. In some aspects, the electrolyte is present in the formulation in an amount ranging from about 10⁻² g/L to about 10 g/L. In some aspects, the electrolyte is present in the formulation in an amount ranging from about 10⁻¹ g/L to about 10 g/L. In some aspects, the metabolite scavenger is present in the formulation in an amount ranging from about 10⁻² g/L to about 10² g/L. In some aspects, the formulation comprises the components listed in any one of TABLES 1-4. In some aspects, the formulation consists of the components listed in any one of TABLES 1-4. In some aspects, the formulation comprises the components listed in TABLE 4. In some aspects, the formulation consists of the components listed in TABLE 4. In some aspects, the formulation has a volume from about 5 milliliter (mL) to about 15 mL. In some aspects, the formulation has a volume of at least about 9 mL. In some aspects, a dilution ratio of the biological sample to the formulation in the container is from about 1:20 to about 1:5. In some aspects, the dilution ratio of the biological sample to the formulation in the container is at least about 1:10. In some aspects, the collection device is a scoop, a spatula, or a brush. In some aspects, the at least one microorganism comprises at least one anaerobic microorganism. In some aspects, the at least one microorganism belongs to a genus selected from the group consisting of Anaerotignum sp., Anaerotruncus sp., Candida sp., Prevotella sp., Fusobacteria sp., Bacteroides sp., Parabacteroides sp., Roseburia sp., Erysipelobacteriaceae sp., Enterobacteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and Lactobacillus sp. In some aspects, the biological sample comprises at least two microorganisms, which at least two microorganisms comprise the at least one microorganisms. In some aspects, the biological sample comprises at least five microorganisms, which at least five microorganisms comprise the at least one microorganisms. In some aspects, the biological sample comprises at least ten microorganisms, which at least ten microorganisms comprise the at least one microorganisms. In some aspects, the biological sample is a stool sample. In some aspects, the subject is a mammal. In some aspects, the mammal is a human.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates an example sample tube and packaging material that may be used to store the sample tube prior to sample collection. The sample tube with formulation inside is produced, packaged, and stored under anaerobic conditions, thereby increasing the stability and shelf-life of the formulation.

FIG. 2A illustrates a method for obtaining a stool sample from a subject.

FIG. 2B illustrates a method for obtaining a stool sample from a subject.

FIG. 3 illustrates a processing scheme for an obtained stool sample. The stool sample can be diluted and then divided into different portions for various analyses, storage, and cell culture.

FIG. 4A illustrates a method for analyzing stool samples that were inoculated with a collection formulation (consisted of the components shown in TABLE 4) in air. The stool samples were then stored in the formulation for 72, 96, or 120 hours at room temperature and subsequently cultured.

FIG. 4B illustrates a method for culture recovery from the formulation samples that were stored either at (i) room temperature or, (ii) at room temperature followed by storage at −80° C. for 7 days and thawing.

FIG. 5A illustrates a method for culturing and harvesting cells from serial dilution samples for whole-genome sequencing (WGS).

FIG. 5B illustrates a method for harvesting colonies from serial dilution and high-density growth plates for WGS.

FIG. 6 illustrates a graph showing the effect of media type and cell dilution factor on cell culture recovery.

FIG. 7A illustrates a bar graph showing the observed operational taxonomic units (OTUs) per 10,000 sequences for fresh stool sample and for stool samples stored in the formulation for 72 hours, 96 hours, and 120 hours, respectively.

FIG. 7B illustrates a line graph showing the observed OTUs as a function of sequences per sample for fresh stool sample and for stool samples stored in the formulation for 72 hours, 96 hours, and 120 hours, respectively.

FIG. 7C illustrates a bar graph showing the relative abundance of selected bacterial species (i) a fresh stool sample, and (ii) stool samples that were stored in the formulation for 72 hours, 96 hours, and 120 hours, respectively.

FIG. 7D shows the relative abundance for the selected species Akkermansia muciniphila, Faecalibacterium prausnitzii (both examples for high relative abundance species), and Bacteroides intestinalis (an example for a low relative abundant species) in samples from fresh stool sample and from formulation samples stored for 72, 96, and 120 hours, respectively.

FIG. 8A illustrates a bar graph showing the observed OTUs per 10,000 sequences for cultures grown on BHI agar. The cells for these cultures were obtained from a fresh stool sample or from stool samples stored in the formulation for 1 hour (To control), 72 hours, 96 hours, and 120 hours. Samples were prepared for storage without anaerobic conditions (e.g. in normal air). One set of stored samples were stored at room temperature (abbreviated as “RT”). A second set of stored samples was stored at room temperature for the indicated time, frozen and stored at −80° C. for an additional 7 days, and then thawed (referred to herein as freeze-thawing and abbreviated as “FRZ”). Following storage, samples were brought into an anaerobic chamber, inoculated onto BHI agar, and cultured. The same inoculation and storage procedures was performed for the “RT” and “FRZ” samples described below in FIG. 8B-FIG. 10D.

FIG. 8B illustrates a line graph showing the observed OTUs as a function of sequences per sample for cultures grown on BHI agar, wherein the cells for these cultures were obtained from fresh stool sample and from stool samples stored in the formulation for 1 hour (To control), 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 9A illustrates a bar graph showing the observed OTUs per 10,000 sequences for cultures grown on CHOC agar, wherein the cells for these cultures were obtained from fresh stool sample and from stool samples stored in the formulation for 1 hour (To control), 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 9B illustrates a line graph showing the observed OTUs as a function of sequences per sample for cultures grown on CHOC agar, wherein the cells for these cultures were obtained from fresh stool sample and from stool samples stored in the formulation for 1 hour (To control), 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 10A illustrates a bar graph showing the observed OTUs per 10,000 sequences for cultures grown on YCFAC+B agar, wherein the cells for these cultures were obtained from fresh stool sample and from stool samples stored in the formulation for 1 hour (To control), 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 10B illustrates a line graph showing the observed OTUs as a function of sequences per sample for cultures grown on YCFAC+B agar, wherein the cells for these cultures were obtained from fresh stool sample and from stool samples stored in the formulation for 1 hour (To control), 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 10C illustrates a bar graph showing the observed OTUs per 10,000 sequences for cultures grown on YCFAC+B agar, wherein the cells for these cultures were obtained from fresh stool sample and from stool samples stored in the formulation for 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 10D illustrates a line graph showing the observed OTUs as a function of sequences per sample for cultures grown on high-dilution YCFAC+B agar, wherein the cells for these cultures were obtained from fresh stool sample and from stool samples stored in the formulation for 72 hours, 96 hours, and 120 hours, respectively, and wherein the 72-hour, 96-hour, and 120-hour “RT” samples were stored at room temperature only, and the “FRZ” samples were stored for an additional 7 days at −80° C. followed by thawing and culturing.

FIG. 11A illustrates a bar graph showing the relative abundance (in %) of the top 20 most abundant species from fresh stool samples and samples after storage in the formulation of TABLE 4 at room temperature for 72 hours, 96 hours, and 120 hours.

FIG. 11B illustrates a bar graph showing the relative abundance (in %) of the middle 20 (i.e., 40-60) of the 100 most abundant species in freshly collected stool sample, as well as in stool sample that was stored in the formulation of TABLE 4 at room temperature for 72 hours, 96 hours, and 120 hours, respectively.

FIG. 11C illustrates a bar graph showing the relative abundance (in %) of the bottom 20 (i.e., 80-100) of the 100 most abundant species in freshly collected stool sample, as well as in stool sample that was stored in the formulation of TABLE 4 at room temperature for 72 hours, 96 hours, and 120 hours, respectively.

FIG. 11D illustrates a bar graph showing the number of abundant (>0.10%), low abundant (0.01%-0.1%) and rare (<0.01%) species recovered from culture combined across all media types for fresh stool sample and for stool sample that was stored in the formulation at room temperature for 72 hours, 96 hours, and 120 hours, respectively.

FIG. 11E illustrates a bar graph showing the number of recovered species with a relative abundance of >0.05% cultured from (i) fresh sample and (ii) from stool samples stored in the formulation shown in TABLE 4 for 72, 96, and 120 hours, respectively, for all four culturing conditions (BHI, CHOC, YCFAC+B, and CFU YCFAC+B media and growth conditions).

FIG. 12 illustrates a method for preparing, culturing and harvesting colonies from growth plates of different cell culture conditions for whole-genome sequencing (WGS).

FIG. 13 illustrates a method for preparing growth plates for fresh stool and T₀ control samples stored in the formulation (formulation composition described in TABLE 4) using BHI, YCFAC+B, and CHOC growth media.

FIG. 14 illustrates a bar graph showing the total number of bacterial species recovered different relative abundance ranges (i.e., <0.001%, 0.001%-0.1%, and >0.1%) from stool samples that were stored in the formulation shown in TABLE 4 at (i) room temperature for 72, 96, and 120 hours, respectively, and (ii) at room temperature for 72, 96, and 120 hours, respectively, followed by storage at −80° C. for 7 days, thawing, and analysis.

FIG. 15A shows the number of species detected in various abundance ranges in a fresh stool sample from Donor 1 compared to samples from Donor 1 that were stored for 72 or 120 hours in the formulation shown in TABLE 4, indicating that the sample itself did not undergo major changes during storage.

FIG. 15B shows the number of species detected in various abundance ranges in a fresh stool sample from Donor 2 compared to samples from Donor 2 that were stored for 72 or 120 hours in the formulation shown in TABLE 4, indicating that the sample itself did not undergo major changes during storage.

FIG. 15C shows the number of species detected in various abundance ranges in a fresh stool sample from Donor 3 compared to samples from Donor 3 that were stored for 72 or 120 hours in the formulation shown in TABLE 4, indicating that the sample itself did not undergo major changes during storage.

FIG. 16 shows the number of recovered species from stool using different culture media.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The term “isolated,” as used herein in the context of a microorganism (e.g., a bacterium, a fungus, e.g., yeast, etc.), generally refers to a microorganism that has been (1) separated from at least some of the components with which it was associated when initially produced (whether in nature or in an experimental setting), and/or (2) produced, prepared, purified, and/or manufactured, e.g. using artificial culture conditions such as (but not limited to) culturing on a plate and/or in a fermenter. In some examples, isolated bacteria can include those bacteria that are cultured, even if such cultures are not monocultures. Isolated bacteria can be separated from at least about 10%, about 20%, about 30%, about 40%, about 50%, about 60%, about 70%, about 80%, about 90%, or more of the other components with which they were initially associated. Isolated bacteria can be more than about 80%, about 85%, about 90%, about 91%, about 92%, about 93%, about 94%, about 95%, about 96%, about 97%, about 98%, about 99%, or more than about 99% pure. A bacterial population of a biological sample provided herein can comprise one or more bacteria, which may then be isolated from such sample.

The term “viable,” as used herein in the context of a microorganism (e.g., a bacterium, a fungus, etc.), generally refers to a microorganism (e.g., bacterium) that is alive, shows healthy vital signs (e.g., shape, form, structure, mobility, etc.), is capable of performing its metabolic activity(ies), and is capable of growing on a cell culture medium.

The term “preserving the viability,” as used herein in the context of microorganisms, generally refers to the ability to keep at least a minimum number of microorganisms of a sample alive to enable their growth or regrowth (e.g., in a cell culture laboratory). In an example, preserving at least about 70% viability of a microorganism in a formulation and for a certain period of time (e.g., 1, 2, 3, 4, 5, or more days) can be defined herein as the ability to grow at least about 70% of the microorganism in a cell culture after storage in the formulation for the given period of time, as compared to a number (100%) of microorganism cells that grow in that cell culture when taken directly from a fresh biological sample without storage, or with only minimal (e.g., 1 hour) storage in the formulation. Thus, an abundance of a microorganism of at least about 70% can be preserved. In another example, a stool sample comprising at least one species of anaerobic bacteria is obtained using the methods, compositions, and kits described herein. The anaerobic bacteria's viability is preserved (or maintained) during transport of the sample from the site of sample collection to the processing unit. Thus, the preserved viability of the anaerobic bacteria in the sample enables regrowth of the anaerobic bacteria, e.g., in a laboratory, after multiple days (e.g., 4, 5, 6, or 10 days) in transit. In some instances herein, a plurality of species, e.g., a plurality of bacterial species present in a biological sample such as a stool sample, can be referred to as “operational taxonomic units” or “OTUs”.

The term “subject,” as used herein, generally refers to an individual, such as a member of the animal kingdom. The subject may be living or not living. The subject may be a human. The subject may have a certain lifestyle, diet, state of health, etc. The subject may be a healthy subject, or not suffering from a disease or disorder. Alternatively, the subject may be suffering from or may be suspected of suffering from a disease or disorder. The subject may be symptomatic of a disease or disorder. Alternatively, the subject may not be symptomatic with respect to the disease or disorder. The subject may have a particular diet (e.g., ketogenic, vegetarian, vegan, pescatarian, etc.). The Subject may have a certain lifestyle (e.g., long distance runner, cross fit, sedentary lifestyle, drug abuse (e.g., alcohol, nicotine, or recreational drugs), etc.). The subject may be from a specific geographic region (e.g., Northern/Western Europe, North America, amazon, Nordic/Scandinavian countries, Africa, Asia, etc.). The subject may have one or more characteristics which may be analyzed and/or correlated with the subject's microbiome composition (e.g., using the herein described methods, compositions, and/or kits), wherein data derived from such analyses may be compared to data derived from another subject (e.g., a subject having the same or different characteristics).

The subject can be a member of a species comprising individuals who naturally suffer from the disease. The subject can be a mammal. Non-limiting examples of mammals can include rodents (e.g., mice and rats), primates (e.g., lemurs, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. The subject can be a human. The subject can be a non-mammalian animal such as a turkey, a duck, or a chicken. The subject can be a farm animal (e.g., pig, goat or cow). The subject can be a living organism suffering from or prone to a disease or condition that can be diagnosed and/or treated using the kits, methods, and systems as provided herein. The subject can provide a biological sample (e.g., a stool sample or blood sample) which can be collected, transported, stored, cultured and/or analyzed using the kits, methods, devices and systems provided herein. The subject may be a patient being treated or monitored by a healthcare provider (e.g., a primary care physician). Alternatively, the subject may not be a patient.

The terms “disease” and “condition,” as used herein, can be used interchangeably and generally refer to a state of being or health status of a patient or subject capable of being diagnosed and/or treated with a kit, method, device, system, and/or composition disclosed herein. The disease can be an inflammatory disease, an infectious disease, or an autoimmune disease. The disease can be associated with or suspected of being associated with a microorganism. The disease can be associated with a microbiome of a subject, such as a gut (e.g., intestinal) microbiome. The disease can be a dysbiosis, such as gut dysbiosis. The disease can be any condition associated with microbial imbalance or dysbiosis, such as inflammation, infection, autoimmune disease, or cancer.

The term “dysbiosis,” as used herein, generally refers to a difference in the gastrointestinal microbiota compared to a healthy or general population. Dysbiosis can comprise a difference in gastrointestinal microbiota commensal species diversity compared to a healthy or general population. Dysbiosis can also comprise a decrease of beneficial microorganisms and/or increase of pathobionts (pathogenic or potentially pathogenic microorganisms) and/or decrease of overall microbiota species diversity. Many factors can harm the beneficial members of the intestinal microbiota leading to dysbiosis, including (but not limited to) antibiotic use, psychological and physical stress, radiation, and dietary changes. Dysbiosis can comprise or promote the overgrowth of a bacterial opportunistic pathogen such as Enterococcus faecalis, Enterococcus faecium, or Clostridium difficile. A dysbiosis can comprise a reduced amount (absolute number or proportion of the total microbial population) of bacterial or fungal cells of a species or genus (e.g., 5%, 10%, 15%, 20%, 25%, 30%, 35%, 40%, 45%, 50%, 55%, 60%, 65%, 70%, 75%, 80%, 85%, 90%, 95% or more lower) compared to a healthy subject (e.g., a corresponding subject who does not have an inflammatory disease, an infection, and who has not been administered an antibiotic within about 1, 2, 3, 4, 5, or 6 months, and/or compared to a healthy or general population). The dysbiosis can comprise an increased amount (absolute number or proportion of the total microbial population) of bacterial or fungal cells within a species or genus (e.g., 5%, 10%, 15%, 20%, 25%, 30%, 35%, 40%, 45%, 50%, 55%, 60%, 65%, 70%, 75%, 80%, 85%, 90%, 95% or more higher) compared to a healthy subject (e.g., a corresponding subject who does not have an inflammatory disease, an infection, and who has not been administered an antibiotic within about 1, 2, 3, 4, 5, or 6 months, and/or compared to a healthy or general population). A subject who comprises a gastrointestinal infection, gastrointestinal inflammation, diarrhea, colitis, or who has received an antibiotic within about 1, 2, 3, 4, 5, 6, 7, 8, 9, or 10 weeks can be deemed to comprise dysbiosis. An impaired microbiota can comprise small intestinal bacterial or fungal overgrowth. Antibiotic administration (e.g., systemically, such as by intravenous injection or orally) can be a common and significant cause of major alterations in the normal microbiota. Thus, as used herein, the term “antibiotic-induced dysbiosis” can refer to dysbiosis caused by or following the administration of an antibiotic. The kits, methods, and systems provided herein can be used to diagnose and/or treat a dysbiosis or a disease or condition associated with dysbiosis in a subject.

The term “diagnosis,” as used herein, generally refers to a relative probability that a disease (e.g., an autoimmune, inflammatory autoimmune, cancer, infectious, immune, dysbiosis, etc.) can be present in a subject. Similarly, the term “prognosis” generally refers to a relative probability that a certain future outcome may occur in the subject with respect to a disease state. The kits, methods, and systems provided herein can be used to diagnose a disease or condition such as a dysbiosis or an infection (e.g., the presence of one or more pathogenic microorganisms) in a subject.

The terms “biological sample” or “sample,” as used herein, can be used interchangeably and generally refer to materials obtained from or derived from a subject (e.g., a human). A biological sample can include sections of tissues such as biopsy and autopsy samples, and frozen sections taken for histological purposes. Such samples can include bodily fluids such as blood and blood fractions or products (e.g., serum, plasma, platelets, red blood cells, and the like), stool and stool fractions or products (e.g., fecal water, such as but not limited to fecal water separated from other fecal components and solids by methods such as centrifugation and filtration), sputum, tissue, cultured cells (e.g., primary cultures, explants, and transformed cells), stool, urine, synovial fluid, joint tissue, synovial tissue, synoviocytes, fibroblast-like synoviocytes, macrophage-like synoviocytes, immune cells, hematopoietic cells, fibroblasts, macrophages, dendritic cells, T-cells, etc. A sample can be obtained from a eukaryotic organism, such as a mammal such as a primate e.g., chimpanzee or human, cow, dog; cat, a rodent, e.g., guinea pig, rat, mouse; rabbit, or a bird, reptile, or fish.

The abbreviation “sp.” for species, as used herein, generally refers to at least one species (e.g., 1, 2, 3, 4, 5, or more species) of the indicated genus. The abbreviation “spp.” for species, as used herein, generally refers to 2 or more species (e.g., 2, 3, 4, 5, 6, 7, 8, 9, 10 or more) of the indicated genus. Kits, devices, systems and compositions provided herein can comprise a single species within an indicated genus or indicated genera, or 2 or more (e.g., a plurality comprising more than 2) species within an indicated genus or indicated genera.

The term “anaerobic,” as used herein, generally refers to low oxygen (O₂) content. An anaerobic environment, for instance, may have an oxygen content that is less than the content of oxygen in the atmosphere. For example, if at sea level a concentration (or partial pressure) of oxygen is 20.95%, an anaerobic environment may have a concentration of oxygen that is less than 20.95%, 20%, 15%, 10%, 5%, 4%, 3%, 2%, 1%, 0.5%, 0.1%, 0.01%, 0.001%, or less. The anaerobic environment may be configured to maintain the concentration of oxygen under anaerobic (e.g., minimal or no detectable oxygen) conditions.

An anaerobic microorganism may be sensitive to oxygen. The sensitivity to oxygen can be exhibited as a lethal effect on the microorganism, or by modification of the growth characteristics or by inhibition of growth of the microorganism. Microorganisms may differ in their sensitivity to oxygen such that a continuous spectrum of oxygen tolerance from the most sensitive, strict anaerobe to the least sensitive hyper aerobe is observed. Tolerance of air at one atmosphere (e.g., “1 atm”) containing approximately twenty percent oxygen represents but one line drawn on this spectrum of oxygen sensitivity, which extends below, as well as above, this particular concentration of oxygen. When a formulation contains an anaerobic microorganism, the dissolved oxygen concentration or the dissolved oxygen tension can be referred to in viewing this spectrum of oxygen sensitivity. For strict anaerobic microorganisms even very low concentrations (less than 10 micromoles per liter (μM)) of dissolved oxygen tension are inhibitory to growth. Examples of anaerobic bacterial microorganisms include, but are not limited to Treponema spp., such as T. denticola; Selenomonas spp., such as S. ruminatum; Clostridium spp., such as C. perfringens; Bacteroides spp., such as B. fragilis; Peptostreptococcus spp., such as P. anaerobius; Eubacterium spp., such as E. lentum; Peptococus spp., such as Pc. assaccharolyticus; Veillonella spp., such as V. parvula, Propionibacterium spp., such as Pr. acnes; Actinomyces spp., such as A. israelii, and Fusobacterium spp, such as F. necrophorum.

The term “scavenger,” as used herein, generally refers to a substance which can be capable of reducing a level, amount, and/or concentration of a certain atom, molecule or chemical compound such as oxygen, peroxides (e.g., oxygen scavenger), and/or cellular metabolites (e.g., a metabolite scavenger) such as acids (e.g., fatty acids, amino acids, and/or derivatives thereof) or reactive oxygen species (i.e., ROSs). Reduction of the level, amount, and/or concentration of such an atom, molecule or chemical compound can be due to enzymatic reaction and/or modification, absorption, irreversible binding, etc.

The term “oxygen-free,” as used herein in the context of a sample collection formulation or a container, generally refers to an oxygen concentration in the sample collection formulation or the container that is less than the concentration of oxygen under atmospheric conditions. In some examples, a formulation or container that is oxygen free has an oxygen content of at most about 300 parts per million (ppm).

The term “impacted,” as used herein refers to a microbial species whose relative abundance in culture after storage in a formulation is reduced by at least 90% (“1-factor impacted”) or at least 99% (“2-factor impacted”) compared to its relative abundance in culture from fresh stool.

The term “about,” as used herein in the context of a numerical value or range, generally refers to ±10% of the numerical value or range recited or claimed, unless otherwise specified.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The present disclosure provides systems, methods, compositions and kits for collecting, transporting, storing, culturing and/or analyzing a biological sample. Such biological sample can be or can comprise a stool sample obtained from a subject (e.g., a human). The biological sample can comprise one or more microorganisms. Such microorganisms can be bacteria, such as different genera, species, and/or strains of bacteria. The biological sample can comprise one or more anaerobic microorganisms, such as anaerobic bacteria. The anaerobic bacteria can be strict and/or facultative anaerobic bacteria. Anaerobic microorganisms, such as anaerobic bacteria, may not be able to remain viable and/or metabolize in a presence of oxygen (e.g., O₂), and may thus prefer environments and/or atmospheres comprising reduced or no detectable oxygen concentrations compared to, e.g., the oxygen concentration of air at sea level.

The present disclosure provides systems, methods, compositions and kits for collecting, transporting, storing, culturing, growing and/or analyzing a biological sample that can comprise at least one anaerobic microorganism. The kits, systems and methods disclosed herein may allow for anaerobic microorganisms to remain viable after sample collection and/or during transport to and storage at a processing or analysis facility. Specifically, the kits, methods, and systems of the present disclosure may allow not only for obtaining a biological sample (e.g., a human stool sample) for analysis (e.g., genetic sequencing) but may also allow for storage (e.g., long-term storage at low temperatures) and, most importantly, for culturing microorganisms of the biological sample following sample collection and/or transport. This may allow for further, more in-depth studies and analyses of the microorganisms present in the biological sample. For example, the ability to culture and grow microorganisms obtained from a broad variety of biological samples may enable the development and production of various analysis platforms such as in vitro assays, animal models, and/or therapeutics such as synthetic stool preparations using the preserved microorganisms of the biological sample(s).

### Formulations

Provided herein are formulations for collecting, transporting and/or storing a biological sample. The biological sample can comprise a stool sample obtained from a subject (e.g., a human). The biological sample can comprise one or more microorganisms. Such microorganisms can be bacteria, such as different genera, species, and/or strains of bacteria. The biological sample can comprise one or more anaerobic microorganisms, such as anaerobic bacteria. The anaerobic bacteria can be strict and/or facultative anaerobic bacteria.

The formulations of this disclosure can provide anaerobic conditions for microorganisms (e.g., anaerobic microorganisms) of a biological sample to extend the time during which the cells remain viable. A formulation can be used to transport and/or store a biological sample that can comprise at least one anaerobic microorganism. Such formulation can allow for anaerobic microorganisms to remain viable after sample collection and during transport and storage to a processing or analysis facility. Thus, the formulations herein may allow for transporting a biological sample (e.g., a human stool sample) to, e.g., a testing facility for, e.g., analysis (e.g., genetic sequencing) but also allows for storage. Such storage can be at room temperature for 1, 2, 3, 4, 5, 6, or more days (e.g., consecutive days), and/or long-term storage of multiple days, weeks, or months at low (e.g., <50° C. or <0° C.) temperatures. Since a formulation herein is capable of preserving the viability of microorganisms present in a biological sample, such microorganisms may be grown using cell culture once the biological sample arrives at a destination, such as a processing and/or analysis facility. This may allow for further studies and analyses of the microorganisms present in the biological sample, and may also enable the development and production of in vitro assays, animal models, and/or therapeutics such as synthetic stool preparations.

Provided herein are formulations that can (i) reduce a sample's exposure to oxygen and metabolic byproducts; (ii) reduce overgrowth of organisms; (iii) have practical storage and shelf life requirements; (iv) maintain organism viability over a range of temperatures during transport; (v) maintain organism viability over an extended period of time; and (vi) preserve organisms for recovery when frozen for storage. Moreover, the herein described methods, compositions, and kits may allow the preservation of the microorganisms' viability under practical and user-friendly conditions. Such practical and user-friendly conditions include sample collection and storage at room temperature (e.g., without freezing) and sample transport using regular transit times (e.g., without rush transport and associated limitations) due to efficient preservation of the microorganism's viability for, e.g., 2, 3, 4, 5, 6, 7, 8, 9, 10 or more days.

Thus, microorganisms present in the biological sample can be (i) analyzed (e.g., sequenced) and (ii) grown in a culture. The culture may be a bacterial cell culture. The ability to establish cultures based on the obtained biological sample (e.g., a human stool sample) can be a particular advantage provided by the present disclosure over conventional sample collection and storage methodologies. It was surprisingly found that the herein disclosed kits, methods, and systems involving the use of various components can allow for efficient and user-friendly sample collection while keeping the microorganisms viable during collection, transport (e.g., for up to, or more than 7 days), and transfer into an anaerobic environment, such as an anaerobic chamber, a glove box, etc. (e.g., at a processing or analysis facility). The kits, devices, and systems provided herein can comprise one or more of any one of a container (e.g., a sample collection tube, and may include a sealable lid and a scoop or different collector), antioxidant, oxygen scavenger, metabolite scavenger (e.g., to remove toxic metabolite and reduce accumulation of metabolites), electrolyte, cryopreservant, and one or more solvents. Thus, the kits, devices, and systems of the present disclosure can comprise various components that, in combination, e.g., in a formulation, can give rise to the herein described advantages compared to conventional systems for collecting biological samples (e.g., stool samples comprising one or more anaerobic microorganisms).

The kits, devices, and systems described herein can comprise any one or more of the formulations described herein. Such formulations can comprise various component(s) and compound(s) having certain functions, e.g., preserving an anaerobic environments through antioxidant or oxygen-scavenging properties, scavenging excess microbial metabolites accumulating in a container, pH control of the formulation, nutrition supply for microorganisms, etc.

Such a formulation can comprise various compounds with antioxidant properties. Such antioxidant compounds can include, but are not limited to, ascorbic acid, dithiothreitol (e.g., DL-dithiothreitol, which may also be referred to herein as “DTT” and which may also be used as a preservative for enzymes, if present in the formulation), glutathione, phenolic acids (e.g., gallic, protochatechuic, caffeic, and rosmarinic acids), phenolic diterpenes (e.g., carnosol and carnosic acid), flavonoids (e.g., quercetin and catechin), volatile oils (e.g., eugenol, carvacrol, thymol, and menthol), α-Tocopherol (e.g., vitamin E), Trolox, ascorbic acid, vitamin A, vitamin C, coenzyme Q10, manganese, iodide, melatonin, alpha-carotene, astaxanthin, beta-carotene, canthaxanthin, cryptoxanthin, lutein, lycopene, zeaxanthin, flavonoids (e.g., flavones such as apigentin), luteolin, tangeithin, flavonols, isorhamnetin, kaempferol, myricetin, proanthocyanidins, quercetin, eriodictyol, hesperetin, naringenin, catechin, gallocatechin, epicatechin, epigallocatechin, theaflavin, thearubigins, isoflavone phytoestrogens, daidzein, genistein, glycitein, stilbenoids such as resveratrol, pterostilbene, anthocyanins, cyanidin, delphinidin, malvidin, pelargonidin, peonidin, petunidin, chicoric acid, chlorogenic acid, cinnamic acid, ellagic acid, ellagitannins, gallic acid, gallotannins, rosmarinic acid, curcumin, xanthones, capsaicin, bilirubin, citric acid, oxalic acid, phytic acid, N-acetylcysteine, R-α-lipoic acid, anthocyanins, copper, cryptoxanthins, flavonoids, indoles, isoflavonoids, lignans, selenium, zinc, any functional derivative, and/or any combination thereof. In some instances, the antioxidant present in a formulation is an ascorbic acid. In other cases, the antioxidant present in a formulation is glutathione.

The formulations used in combination with the kits, devices, and systems described herein can comprise various compounds with oxygen (e.g., molecular oxygen O2, peroxides, superoxides, hydroxyl radical, single oxygen, or alpha-oxygen) scavenging properties. Such oxygen scavengers can include, but are not limited to, ferrous carbonate, ascorbic acid (including e.g., D- or L-ascorbic acid, their alkali and alkaline earth metal salts, analogues such as erythorbic acid and mixtures thereof), sodium thioglycolate and derivatives thereof, cysteine (e.g., L-cysteine), t-butyl hydroquinone, butylated hydroxytoluene, sulfites and analogues, boron/boric acid and analogues, sugar alcohols (e.g., xylitol, sorbitol, mannitol), 1,2-glycols (e.g., propylene or ethylene glycol), unsaturated fatty acids (e.g., linseed oil), hydrocarbons (e.g., isoprene, butadiene, and squalene), palladium catalysts, hydrogen gas, glucose oxidase, immobilized yeast, and/or any metal reactive with oxygen such iron, zinc, and/or manganese, or any combination thereof. Oxygen scavengers used in combination with the herein disclosed kits, methods, and systems can be enzymes. Such enzyme(s) that can be part of a formulation herein can include a peroxidase (e.g., glutathione peroxidase), a dismutase (e.g., superoxide dismutase), a catalase, or any combination thereof. Such enzymes, either alone or in combination, may be present in a collection formulation in amounts ranging from about 10 units (U) to about 1,000,000 U. The amount of an enzyme in a collection formulation may be from about 10 U to about 150,000 U. The amount of an enzyme in a collection formulation may be from about 50 U to about 100,000 U. The amount of an enzyme in a collection formulation may be from about 500 U to about 50,000 U. The amount of an enzyme in a collection formulation may be from about 1,000 U to about 10,000 U. The enzyme unit (“U”) is defined herein as the amount of enzyme that catalyzes the conversion of one micromole of substrate per minute.

The formulations used in combination with the kits, devices, and systems described herein can comprise various compounds that can scavenge (e.g., bind, absorb, modify, transform, etc.) metabolites that can be produced by microorganisms during transport and/or storage, and which compounds can be and/or become toxic to microorganisms, e.g., when these compounds accumulate (e.g., inside the container). Such metabolite scavengers can include, but are not limited to, activated carbon, charcoal, ascorbic acid, tocopherol, naringenin, organotin compounds, glutathione, and/or metallic nanoparticles, or any derivative or combination thereof. In some instances, a formulation comprises activated carbon.

The formulations used in combination with the kits, devices, and systems described herein can comprise various electrolytes. Such electrolytes can include, but are not limited to, sodium chloride, potassium chloride, sodium phosphate, magnesium sulfate, hydrochloric acid, nitric acid, sulfuric acid, hydrobromic acid, hydroiodic acid, perchloric acid, acetic acid, carbonic acid, ammonia, lithium hydroxide, sodium hydroxide, potassium hydroxide, rubidium hydroxide, cesium hydroxide, calcium hydroxide, strontium hydroxide, barium hydroxide, sodium nitrate, copper sulphate, calcium chloride, sodium bicarbonate, zinc sulphate, any derivative, and/or any combination (e.g., salt and/or counter ion combination such as sodium chloride and magnesium chloride) thereof.

The formulations used in combination with the kits, devices, and systems herein can comprise various cryopreservants such as glycerol, dimethyl sulfoxide (DMSO), 1,2-propanediol, 2-methyl-2,4-pentanediol, polyvinyl pyrrolidone, hydroxyethyl starch, various sugars, ethylene glycol, a derivative thereof, or a combination thereof. In some cases, the cryopreservant is glycerol.

The formulations used in combination with the kits, devices, and systems provided herein can comprise media and solutions for collecting, transporting, storing, culturing and/or analyzing/processing biological samples that can comprise one or more components as described herein, e.g., those listed in any one of TABLES 1-4 in EXAMPLE 1 herein. Any one of such components, e.g., electrolytes, oxygen scavengers, metabolite scavengers, antioxidants, reducing agents, cryopreservants, etc., can be present in various amounts or concentrations. The concentration of any of such components in a formulation described herein can be from about 10⁻¹⁰ moles per liter (mol/L) to about 10 mol/L. The concentration of any of such components in such a formulation can be from about 10⁻⁵ mol/L to about 1 mol/L. The concentration of any of such components in a formulation can be from about 10⁻³ mol/L to about 10⁻¹ mol/L. The concentration of any of such components in a formulation can be from about 10⁻² mol/L to about 10⁻¹ mol/L. The concentration of any of such components in a formulation can be from about 0.1 mol/L to about 0.25 mol/L. In some cases, the concentration of any of such components in such a formulation can be from about 10⁻⁵ g/L to about 500 g/L. The concentration of any of such components in a formulation can be from about 10⁻³ g/L to about 10² g/L. The concentration of any of such components in a formulation can be from about 10⁻² g/L to about 10¹ g/L. The concentration of any of such components in a formulation can be from about 0.1 g/L to about 0.25 g/L, from about 0.1 g/L to about 1 g/L, from about 1 g/L to about 10 g/L, from about 10 g/L to about 10² g/L, or from about 10² g/L to about 10³ g/L.

Provided herein are compositions, kits, devices, and systems that can comprise a collection formulation (e.g., an oxygen-free, pre-reduced collection formulation) comprising any one or more of the herein described antioxidant(s), oxygen scavenger(s), metabolite scavenger(s) (e.g., to remove toxic metabolite and reduce accumulation of metabolites), electrolyte(s), cryopreservant(s), and/or solvent(s). In an example, a collection formulation as described herein can comprise one or more of the electrolytes sodium phosphate, sodium chloride, potassium chloride, potassium phosphate, and/or magnesium sulfate, activated carbon as metabolite scavenger, one or more of the antioxidants: peroxidase, ascorbic acid, glutathione, and a dithiothreitol, and/or one or more of the oxygen scavengers: sodium thioglycolate, L-cysteine, peroxidase, catalase, and dismutase. The concentration of electrolyte(s) in a collection formulation, either alone or in combination, may be from about 0.01 g/L to about 15 g/L, from about 0.1 g/L to about 5 g/L, or from about 0.1 g/L to about 1.5 g/L. The concentration of metabolite scavenger(s) in a collection formulation, either alone or in combination, may be from about 0.2 g/L to about 2 g/L. The concentration of oxygen scavengers, e.g., small molecule oxygen scavengers (e.g., sodium thioglycolate), in a collection formulation, either alone or in combination, may be from about 0.2 g/L to about 2 g/L. The amount of oxygen scavenging enzymes in a collection formulation, either alone or in combination, may be from about 10 U to about 10⁶ U. Example compositions of collection formulations described herein are shown in TABLES 1-4.

The pH of a formulation provided herein can be from about 4 to about 9. The pH of a formulation can be from about 5 to about 8. The pH of a formulation can be from about 6 to about 8. The pH of a formulation can be from about 6.5 to about 7.8. The pH of a formulation can be from about 6.8 to about 7.7. The pH of a formulation can be from about 6.9 to about 7.6. The pH of a formulation can be from about 7 to about 7.5. The pH of a formulation can be buffered by, e.g., using a phosphate and/or carbonate buffer or any other buffer components (e.g., zeolites).

The present disclosure provides kits, devices, and systems that can comprise various oxygen concentrations inside a sample container (e.g., a sample tube). A sample tube or sample container of the present disclosure can comprise a formulation as a microbial anaerobic collection formulation that can occupy a certain volume of the tube or container (e.g., about 75% of the volume, such as 9 milliliters (mL) formulation in a 12 mL tube). Both, the formulation as well as the gaseous space (or gaseous medium) within a tube or container can have certain concentrations (e.g., in a formulation) or certain partial pressures (e.g., in a gaseous space) of oxygen (e.g., molecular oxygen or O₂).

The volume of an anaerobic collection formulation used with the herein described kits, methods, and systems can vary depending on intended use. A volume of about 1 mL to about 50 mL of formulation can be used to achieve a certain sample-formulation concentration or ratio (e.g., 1:10 if 1 gram (g) sample is added to 9 mL of formulation). The volume of a formulation (e.g., in a sample collection container) can be from about 1 mL to about 5 mL. The volume of a formulation can be from about 5 mL to about 10 mL. The volume of a formulation can be from about 10 mL to about 15 mL. The volume of a formulation can be from about 10 mL to about 25 mL. The volume of a formulation can be from about 20 mL to about 50 mL. The volume of a formulation can be at least about 1 mL. The volume of a formulation can be at least about 2 mL. The volume of a formulation can be at least about 3 mL. The volume of a formulation can be at least about 5 mL. The volume of a formulation can be at least about 9 mL. The volume of a formulation can be at least about 10 mL. The volume of a formulation can be at least about 14 mL. The volume of a formulation can be at least about 15 mL. The volume of a formulation can be at least about 20 mL.

The volume of a gaseous space (also referred to herein as “head space”) used with the herein described kits, methods, and systems can vary depending on intended use. The volume of a gaseous space (e.g., in a sample collection container) can be from about 1 mL to about 5 mL. The volume of a gaseous space can be from about 5 mL to about 10 mL. The volume of a gaseous space can be from about 10 mL to about 15 mL. The volume of a gaseous space can be from about 10 mL to about 25 mL. The volume of a gaseous space can be from about 20 mL to about 50 mL. The volume of a gaseous space can be at least about 1 mL. The volume of a gaseous space can be at least about 2 mL. The volume of a gaseous space can be at least about 3 mL. The volume of a gaseous space can be at least about 5 mL. The volume of a gaseous space can be at least about 9 mL. The volume of a gaseous space can be at least about 10 mL. The volume of a gaseous space can be at least about 14 mL. The volume of a gaseous space can be at least about 15 mL.

The volume of a formulation can be at least about 5%, 10%, 15%, 20%, 25%, 30%, 35%, 40%, 45%, 50%, 55%, 60%, 65%, 70%, 75%, 80%, 85%, 90%, or 95% of the volume of a sample collection container (e.g., a collection tube), when such container is sealed. In such instances, the volume of a formulation can be about 60%, 70%, 75%, 80%, or 85% of the volume of a sample collection container. In an example, a sample collection tube having a volume of 12 mL can contain about 9 mL of formulation and about 3 mL of gaseous space. In another example, a sample collection tube having a volume of 12 mL can contain about 10 mL of formulation and about 2 mL of gaseous space. In some instances, the combined volume of a formulation and a biological sample can be at least about 5%, 10%, 15%, 20%, 25%, 30%, 35%, 40%, 45%, 50%, 55%, 60%, 65%, 70%, 75%, 80%, 85%, 90%, or 95% of the volume of a sample collection container (e.g., a collection tube), when such container is sealed.

The herein described kits, methods, and systems may be used to collect from about 1 mg to about 10 g of sample (e.g., biological sample such as a stool sample). The weight of a collected sample can be at least about 10 mg. The weight of a collected sample can be at least about 50 mg. The weight of a collected sample can be at least about 100 mg. The weight of a collected sample can be at least about 500 mg. The weight of a collected sample can be at least about 1 g. The weight of a collected sample can be at least about 2 g. The weight of a collected sample can be at least about 5 g. The weight of a collected sample can be at least about 10 g.

Thus, the weight or volume ratio of biological sample to formulation (e.g., an anaerobic transport formulation) can range from about 1:1 to about 1:10⁶. The weight or volume ratio of biological sample to formulation can be from about 1:1 to about 1:10³. The weight or volume ratio of biological sample to formulation can be from about 1:10 to about 1:100. The weight or volume ratio of biological sample to formulation can be from about 1:1 to about 1:10. The weight or volume ratio of biological sample to formulation can be about 1:10.

The volume of an anaerobic sample collection formulation used with the herein described kits, methods, and systems may be about 9 mL. The weight of a sample (e.g., a biological sample) that may be collected with a container comprising the about 9 mL of formulation can be about 1 g, resulting in a sample dilution of about 1:10.

The presently described kits, methods, devices and systems can provide and/or maintain an oxygen concentration in a sample container or transport container (e.g., a transport tube) that can be below the atmospheric oxygen concentration of about 20%. The kits, methods, devices and systems described herein can allow for a reduction of the amount or concentration of oxygen in the sample collection container and the liquid sample storage formulation after the sample has been placed into the container. This can be achieved by using one or more antioxidants and/or one or more oxygen scavengers that reduce the amount or concentration of oxygen over time, e.g., during storage or transport and while the container can be closed or sealed.

The oxygen concentration of a collection formulation described herein can be from about 10⁻³ milligram per liter (mg/L) to about 9 mg/L. The oxygen concentration of a collection formulation can be the same before and after sample collection. The oxygen concentration of a collection formulation can be different before and after sample collection. The oxygen concentration of a collection formulation as described before and/or after sample collection can be from about 10⁻² mg/L to about 1 mg/L. The oxygen concentration of a collection formulation before and/or after sample collection herein can be from about 10⁻¹ mg/L to about 0.5 mg/L. The oxygen concentration of a collection formulation before and/or after sample collection can be less than about 9 mg/L. The oxygen concentration of a collection formulation before and/or after sample collection can be less than about 5 mg/L. The oxygen concentration of a collection formulation before and/or after sample collection can be less than about 1 mg/L. The oxygen concentration of a collection formulation before and/or after sample collection can be less than about 0.5 mg/L. The oxygen concentration of a collection formulation before and/or after sample collection can be less than about 10⁻¹ mg/L. The oxygen concentration of a collection formulation before and/or after sample collection can be less than about 10⁻² mg/L. The oxygen concentration of a collection formulation before and/or after sample collection can be less than about 10⁻³ mg/L.

The oxygen (e.g., O₂) concentration in a gaseous space inside a container (e.g., collection tube) can be less than about 21%, 20.95%, 20%, 15%, 10%, 5%, 4%, 3%, 2%, 1%, 0.5%, 0.1%, 0.01%, 0.001%, or less. This may be the case when the container is sealed from an external environment, which may have an oxygen concentration of about 20.95%. The oxygen concentration in a gaseous space inside the container can be the same before and after sample collection. The oxygen concentration in the gaseous space inside the container can be different before and after sample collection. The oxygen concentration in the gaseous space inside the container, subsequent to sample collection, deposition of the collected sample in the container, and sealing of the container can be less than about 20.95%, 20%, 15%, 10%, 5%, 4%, 3%, 2%, 1%, 0.5%, 0.1%, 0.01%, 0.001%, or less. The oxygen concentration in a gaseous space inside the container can be from about 1% to about 20%. The oxygen concentration in a gaseous space inside the container can be from about 2% to about 18%. The oxygen concentration in a gaseous space inside the container can be from about 5% to about 15%. The oxygen concentration in a gaseous space inside the container can be from about 7% to about 12%. The oxygen concentration in a gaseous space inside the container can be from about 7% to about 12%. The oxygen concentration in a gaseous space inside the container can be less than about 21%. The oxygen concentration in a gaseous space inside the container can be less than about 20.95%. The oxygen concentration in a gaseous space inside the container can be less than about 20%. The oxygen concentration in a gaseous space inside the container can be less than about 18%. The oxygen concentration in a gaseous space inside the container can be less than about 15%. The oxygen concentration in a gaseous space inside the container can be less than about 10%. The oxygen concentration in a gaseous space inside the container can be less than about 5%. The oxygen concentration in a gaseous space inside the container can be less than about 2%. The oxygen concentration in a gaseous space inside the container can be less than about 1%. The oxygen concentration in a gaseous space inside the container can be less than about 0.5%.

In an example, a sample (e.g., a biological sample such as a stool sample of a subject) can be deposited in a container comprising an oxygen-free, pre-reduced formulation comprising an oxygen scavenger. Once the container is exposed to air, the concentration of oxygen in the headspace may increase to about 20.95%, but once the sample is deposited in the container and sealed with the formulation, the scavenger may reduce the concentration of oxygen in the container to below about 20.95%, 20%, 15%, 10%, 5%, 4%, 3%, 2%, 1%, 0.5%, 0.1%, 0.01%, 0.001%, or less.

The formulations used in combination with the kits, devices, and systems of the present disclosure can comprise one or more indicators that can provide information when an anaerobic condition is compromised. Such indicator (e.g., a color indicator) can be a compound (e.g., an organic compound) such as an oxygen indicator. Such oxygen indicator can be reversible or irreversible.

The formulations used in combination with the kits, devices, and systems of the present disclosure can comprise components such as oxygen scavenging compounds that can reduce the oxygen concentration in the liquid formulation and/or in the gaseous medium in a sample container (e.g., a sample tube) during transport, e.g., between the time the sample is collected and container is closed and/or sealed and the time the sample is removed from the container.

The formulations used in combination with the methods, kits, devices, and systems of the present disclosure may allow the survival of microorganisms in the presence of some oxygen (e.g., low concentrations of oxygen), through removal of oxygen and/or reactive oxygen species such as hydrogen peroxide, superoxide anion, hydroxyl radical, etc. that may be produced by the microorganisms during aerobic metabolism. The components of a formulation themselves may not impact the viability of microorganisms in a biological sample. As an example, anaerobic bacteria may grow, at least to some extent, aerobically in the presence of some or low amounts of oxygen present in the formulation and/or container (e.g., after the container has been opened to deposit a sample). The aerobic metabolism may result in the production of reactive oxygen species from which the microorganisms are not protected due to a lack of specific enzymes that can scavenge these reactive oxygen species. The presence of oxygen scavenger(s) (e.g., enzymes such as peroxidase, catalase, etc.) and/or antioxidants in the herein described collection formulations may therefore preserve the microorganism's viability in the presence of some oxygen and aerobic metabolism.

The herein described kits, methods, devices and systems can maintain the viability of microorganisms at various temperatures and temperature ranges. Viability can be maintained at temperatures ranging from about −100 degree Celsius (° C.) to about 50° C. Viability can be maintained at temperatures ranging from about −80° C. to about 30° C. Viability can be maintained at temperatures ranging from about −50° C. to about 30° C. Viability can be maintained at temperatures ranging from about −5° C. to about 37° C. Viability can be maintained at temperatures ranging from about 5° C. to about 35° C. Viability can be maintained at temperatures ranging from about 15° C. to about 25° C. Viability can be maintained at temperatures ranging from about 20° C. to about 25° C. Viability can be maintained at temperatures ranging from about 22° C. to about 25° C.

Biological samples to be collected, stored, and/or transported as described herein can be used to prevent, treat, and/or diagnose a disease or condition. Biological samples that can be used in combination with the herein disclosed kits, methods, devices and compositions can be from a subject. The subject can be a human or a non-human animal. The biological samples can be obtained from various body parts of the subjects. The biological samples can be stool samples. A biological sample can contain microorganism that can be used for therapeutic and/or diagnostic purposes. For example, microorganisms from a stool sample can be purified, cultured, and enriched for certain bacterial species or strains and can then be re-administered to a subject to prevent and/or treat a dysbiosis, such as a dysbiosis of a gut microbiome. As another example, microorganisms from a biological sample can be used to diagnose a disease or condition, such as an infection (e.g., a Clostridium difficile mediated infection). Thus, the herein described kits, methods, devices and systems can be used to identify the presence of one or more pathogenic microorganisms in a biological sample (e.g., a sample provided by a human subject).

The herein described kits, methods, and systems can be used to maintain the viability of various microorganisms that can be present in a biological sample. Such microorganisms include bacteria, yeast, fungi, protozoa, viruses, etc. A biological sample of the present disclosure can comprise one or more anaerobic bacteria (e.g., strict and/or facultative anaerobic), one or more spore-forming bacteria, or any combination of the above and/or the below described bacterial families, genera, species, and/or strains.

A biological sample that may be stored in a formulation can comprise one or more bacterial cells of any one of the bacterial families of Coriobacteriaceae, Bacteroidaceae, Porphyromonadaceae, Bacillaceae, Paenibacillaceae, Lactobacillaceae, Clostridiaceae, Lachnospiraceae, Peptostreptococcaceae, Ruminococcaceae, Clostridiales, Erysipelotrichaceae, Acidaminococcaceae, Veillonellaceae, Enterobacteriaceae, and/or Verrucomicrobiaceae.

A biological sample that may be stored in a formulation can comprise one or more bacterial cells of any one of the bacterial genera of Anaerotignum sp., Anaerotruncus sp., Candida sp., Prevotella sp., Fusobacteria sp., Bacteroides sp., Parabacteroides sp., Roseburia sp., Erysipelobacteriaceae sp Enterob acteriaceae sp., Acidaminococcus sp., Faecalibacterium sp., Enterobacteriaceae sp., Collinsella sp., Eubacterium sp., Lactococcus sp., Lachnospiraceae sp., Gammaproteobacteria sp., Clostridium sp., Clostridium clusters IV and/or XIVa, Pseudoflavinofractor sp., Blautia sp., Dorea sp., Streptococcus sp., and/or Sporanaerobacter sp., Akkermansia sp., Faecalicatena sp., Holdemania sp., Burkholderiales sp., Parabacteriodes sp., Flavonifractor sp., Gordonibacter sp., Parasutterella sp., Bilophila sp., Eggerthella sp., Anaerostipes sp., Coprococcus sp., Alistipes sp., Bifidobacterium sp., and/or Lactobacillus sp.

The bacterial genera, species and/or strains that may be present in a biological sample can comprise one or more anaerobic bacterial genera, species or strains. A biological sample can comprise strain 13LG (Eubacterium limosum), strain 31 FAA (Eubacterium limosum), Faecalibacterium prausnitzii, Bifidobacterium breve, Anaerotruncus colihominis, Anaerotignum lactatifermentans, Burkholderiales finegoldii, Parabacteriodes sp. D26, Prevotella copri, Prevotella sp. Marseille-P4119, Prevotella lascolaii, Roseburia spp., Eubacterium rectale, Bacteroides ovatus, Parabacteriodes distasonis, Eubacterium eligens, Alistipes shahii, Alistipes sp. AL-1, Alistipes onderdonkii, Bilophila wadsworthia, Bilophila sp. 4130, Clostridiales bacterium KLE1615, Faecalicatena contorta, Flavonifractor plautii, Eubacterium ventriosum, Eubacterium hallii, Roseburia spp., Blautia spp., Blautia producta, Dorea spp., Dorea longicatena, Bifidobacterium longum, Bifidobacterium bifidum, Eubacterium hadrum, Eubacterium siraeum, Eubacterium ramulus, Anaerostipes coli, Anaerostipes hadrus, Clostridium aldenense, Clostridium lavalense, Clostridium hathewayi, Clostridium symbiosum, Clostridium orbiscindens, Clostridium sp. SS2/1, Holdemania filiformis, Clostridium citroniae, Clostridium sp. ATCC BAA-442, Clostridium sp. ATCC 29733, Streptococcus thermophilus, Streptococcus salivarius, Streptococcus parasanguinis, Gordonibacter urolithinfaciens, Lachnospiraceae bacterium 3157FAA CT1, Clostridium thermocellum, Ruminococcus obeum, Ruminococcus productus, Ruminococcus gnavus, Bacteroides vulgatus, Bacteroides ovatus, Ruminococcus torques, Ruminococcaceae bacterium D16, Parasutterella excrementihominis, Roseburia inulinovorans, Roseburia intestinalis, Blautia coccoides, Blautia obeum, Coprococcus comes, Blautia wexlerae, Blautia massiliensis, Sutterella sp., Dialister invisus, Akkermansia muciniphila, Collinsella sp. 4847FAA, Collinsella sp. TF06-26, Eggerthella lenta, Prevotellamassilia timonensis, Bifidobacterium pseudocatenulatum, Lactobacillus casei, Lactobacillus johnsonii, and species and/or strains having all of the identifying characteristics of these strains. A sample (e.g., a fecal or stool sample) may comprise any one or more of the bacterial species Fusobacterium nucleatum, Lactobacillus inners, or Clostridioides difficile. In some instances, a formulation herein can preserve the viability and allow cell culture subsequent to transport and/or storage of any one or more of the following species Faecalibacterium prausnitzii, Akkermansia muciniphila, Bifidobacterium spp., and Bacteroides spp.

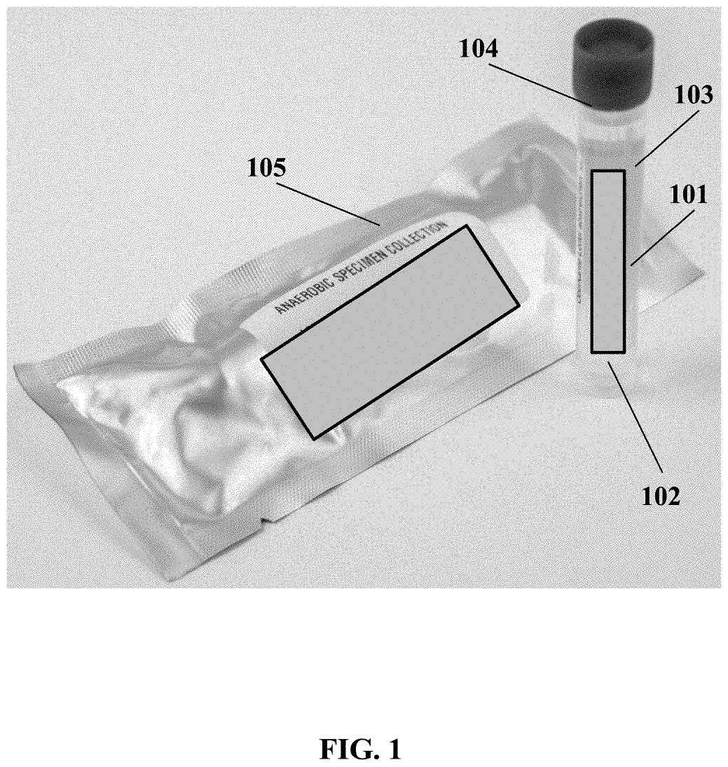

In an example, the devices, kits, and systems of the present disclosure can comprise a container 101 that can comprise a formulation (e.g., a liquid collection or storage formulation) 102 and/or a gaseous medium 103 (FIG. 1). The liquid formulation 102 can comprise at least one electrolyte, at least one antioxidant, at least one oxygen scavenger, and/or at least one metabolite scavenger. The gaseous medium 103 can comprise one or more gases (e.g., inert gases) such as carbon dioxide, nitrogen, hydrogen and/or argon. The container of the present disclosure can comprise a lid 104 that can be used to close and/or seal the container, e.g., following the deposit of a biological sample into the container. Such lid can comprise a collection device such as a scoop that may be attached (e.g., releasably attached) to the lid. The collection device can be used to obtain the biological sample. In an example, the collection device may allow for the collection of a certain amount, volume, and/or weight of the biological sample, e.g., about 1 g of sample. The container 101 can be provided in a packaging 105. Such packaging may protect the container (e.g., during transport to a sample collection site) and/or may allow for maintaining a low-oxygen environment or atmosphere in the container. In an example, the packaging may be filled with a gas such as carbon dioxide, nitrogen, and/or argon in order to provide a low oxygen environment. Thus, provided herein are formulations that can have a shelf-life of at least several weeks or months as further described elsewhere herein.

### Containers

The kits, methods, and systems of the present disclosure can include the use of at least one container. The kits, methods, and systems described herein can include the use of at least 1, 2, 3, 4, 5, 6, 7, 8, 9, 10 or more containers. A container of the present disclosure can be initially sealed, e.g., before a biological sample is deposited in the container. The container as provided herein can initially comprise (e.g., before a biological sample is deposited in the container) a liquid, a gaseous medium, or any combination thereof. The liquid can be an anaerobic collection formulation or formulation as described herein. The gaseous medium can comprise one or more gases. Such gases can include, but are not limited to, carbon dioxide (CO₂), nitrogen (N₂), hydrogen (H₂), argon (Ar), or any combination thereof. Thus, a container can comprise a low oxygen environment. The gaseous medium of a container can comprise one or more gases that are heavier than air (e.g., Ar). This may allow for preserving or maintaining a low oxygen environment in the container even when the container is (e.g., temporarily such as for a few minutes) opened (e.g., by a user) to deposit a biological sample.

In some examples, a container that can be used in combination with the herein described kits, methods, and systems can initially comprise one or more gases that form a low oxygen atmosphere inside the container. At a sample site, a user may open the container, deposit a biological sample and an anaerobic collection formulation as described in the present disclosure into the container (e.g., by using a collection device such as a scoop), close the container, homogenize the sample (e.g., by shaking, mixing, etc.), and seal the container.

The kits, methods, and systems described herein can include the use of a container initially comprising an anaerobic collection formulation. The container comprising the collection formulation can further comprise a gaseous medium as described herein. A user collecting a biological sample may deposit such sample in the collection formulation inside the container. In other examples, the kits, methods, and systems described herein can include the use of a container initially comprising a gaseous medium comprising one or more gases (e.g., carbon dioxide, nitrogen, argon, or a combination thereof). A user collecting a biological sample may deposit the sample in the container, followed by the addition of an anaerobic collection formulation or formulation to the container comprising the sample. Thus, the anaerobic collection formulation may be added to the container prior to, concurrently with, and/or subsequent to depositing the biological sample.

### Collection Devices

The kits, systems and composition herein can comprise a collection device. Such collection device can be used to obtain a sample, e.g., from a subject. In some instances, the collection device can be a scoop or brush. The collection device can be a swap. The collection device can be a spatula such as a plastic spatula. The collection device can be a pipette. The kits, methods, and systems described herein can include the use of at least one collection device. The herein described kits, methods, and systems can include the use of at least 1, 2, 3, 4, 5, 6, 7, 8, 9, 10 or more collections devices. As an alternative, the kit may not include a collection device.

The collection device can be attached to the lid of the container (e.g., a sample tube) and can be used to collect the sample (e.g., a stool sample). The sample can be placed in the container, e.g., by closing and/or sealing (e.g., within 1, 2, 3, or 5 minutes of voiding) the container using the lid that may have the collection device attached to it. Upon sample collection, the container comprising the biological sample can be transported, mailed, and/or delivered to the requesting laboratory facility for further processing and analysis.

### Production

The kits, methods, and systems provided in the present disclosure can be used for the collection, preservation, and/or storage of human and animal anaerobic microbiota samples for the purpose of recovery, growth, and/or isolation of such microorganisms (e.g., bacteria such as anaerobic bacteria).

A transport formulation or collection formulation as described in the present disclosure can be manufactured under specific conditions. Such conditions can include oxygen free and/or anaerobic manufacturing processes. These manufacturing processes can provide conditions wherein a solution can be produced, poured, and/or packaged under oxygen free conditions. Such conditions can aid in the stability and shelf life of the components, and can also provide a pre-reduced environment for the samples to be placed in. Culture media as used and described herein for culturing and processing microorganisms obtained from a biological sample can be produced under similar conditions. Containers (e.g., tubes, test tubes, etc.) and/or packaging that are used in combination with the kits, devices, and systems disclosed herein can be produced under anaerobic conditions to provide an interior atmosphere with reduced oxygen content (or oxygen concentration).

As described herein, a formulation of the present disclosure that can be used for collection, storage and preservation of microorganisms (e.g., bacteria) can contain a buffered mineral salt solution and enzymes in liquid form. Such a formulation can be modified according to an intended use, by adding components that provide optimal survival conditions for anaerobic organisms. Such components (e.g., oxygen scavengers, antioxidants, metabolite scavengers (e.g., to remove toxic metabolite and reduce accumulation of metabolites), etc.) can also reduce the impact of oxygen that a sample may be exposed to prior, during, and/or subsequent to transport. A formulation herein for transport and/or storage of a biological sample comprising microorganisms (e.g., anaerobic bacteria) can be non-nutritive or minimally nutritive to minimize overgrowth during storage and/or transport. As an alternative, a formulation can include one or more nutrients to preserve bacteria during storage and/or transport.

Various methods can be used to produce anaerobic media and sample solutions and to generate anaerobic conditions in a container (e.g., a sample collection tube). Any solution or medium such as anaerobic phosphate buffered saline (PBS) buffer (e.g., including about 0.01% to about 0.05% Tween 80 [vol/vol]) can be freshly prepared by flushing the solution with, e.g., nitrogen or argon. A formulation can be reduced by adding sodium sulfide, e.g., about 0.1 gram per liter (g/liter) to about 0.8 g/liter sodium sulfide.9H₂O. As a redox indicator, sodium resazurin (e.g., about 0.001 g/liter to about 0.001 g/liter) can be added.

For sample collection, any component of a sample collection kit as described herein can be stored in, treated with, or added to a sterile, anaerobic buffer solution (e.g., anaerobic PBS). Such kits can comprise one or more containers comprising an aerobic atmosphere, e.g., comprising nitrogen and carbon dioxide and an anaerobic formulation.

### Storage and Shelf Life

The compositions and kits of the present disclosure can have various storage and shelf-lives while stored at various temperatures. For example, a formulation provided herein can have the advantage of having a shelf-life of several days, weeks, months, or years. Prior to collection of a sample, microbial anaerobic preservation formulation described herein can be stored in its anaerobic packaging between about −5 degree Celsius (° C.) to about 35° C. A formulation described herein can be stored in its anaerobic packaging between about 0° C. to about 25° C. A formulation described herein can be stored in its anaerobic packaging between about 2° C. to about 25° C. A formulation described herein can be stored in its anaerobic packaging between about 5° C. to about 20° C. When stored at such temperatures, the shelf life of a formulation as described herein can be at least about 18 months. The shelf life of a formulation as described herein can be at least about 12 months. The shelf life of a formulation as described herein can be at least about 6 months. The shelf life of a formulation as described herein can be at least about 3 months.

The kits, methods, and systems of the present disclosure can provide collection formulations for collection and/or transporting a biological sample, wherein the microorganisms of the biological sample can be maintained viable for at least about 1 day, 2 days, 3 days 4 days, 5 days, 6 days, 1 week, 2 weeks, 3 weeks, 1 month, 2 months or more. In some examples, the microorganisms of that biological sample can be maintained viable from about 1 day to about 7 days. The presently described kits, methods, and systems can provide viability of microorganisms for about 2 days to about 6 days. The presently described kits, methods, and systems can provide viability of microorganisms for about 3 days to about 5 days. The presently described kits, methods, and systems can provide viability of microorganisms for about 2 days to about 4 days. Viability of microorganisms (e.g., bacteria) can be maintained for at least about 1 day using the presently described kits, methods, and systems. Viability of microorganisms (e.g., bacteria) can be maintained for at least about 2 days using the presently described kits, methods, and systems. Viability of microorganisms (e.g., bacteria) can be maintained for at least about 3 days using the presently described kits, methods, and systems. Viability of microorganisms (e.g., bacteria) can be maintained for at least about 4 days using the presently described kits, methods, and systems. Viability of microorganisms (e.g., bacteria) can be maintained for at least about 5 days using the presently described kits, methods, and systems. Viability of microorganisms (e.g., bacteria) can be maintained for at least about 6 days using the presently described kits, methods, and systems. Viability of microorganisms (e.g., bacteria) can be maintained for at least about 7 days using the presently described kits, methods, and systems. Viability of microorganisms (e.g., bacteria) can be maintained for at least about 8 days using the presently described kits, methods, and systems. Viability of microorganisms (e.g., bacteria) can be maintained for at least about 10 days using the presently described kits, methods, and systems. Such microorganisms can be one or more bacterial species. The bacterial species can be anaerobic bacteria. Such anaerobic bacteria can be strict and/or facultative anaerobic bacteria.

Subsequent to transport, microbiota samples of the present disclosure can be processed (e.g., subjected to sequencing and/or cell culturing) and/or frozen (e.g., at about −80° C.). Thus, the herein disclosed kits, methods, and systems can allow processing, analysis, and culturing of microorganisms of a biological sample that may have been obtained from a subject (e.g., a human).

The formulations of the present disclosure that can be used in the kits, methods, and systems described herein, can be stored for several days, several months, or several years without impacting their ability to preserve microorganisms (e.g., anaerobic bacteria) of a biological sample (e.g., a stool sample). Thus, in some instances, a formulation herein, e.g., any one of the formulations shown in TABLES 1-4, can be stored for at least about 1, 2, 3, 4, 5, 6, 7, 8, 9, or 10 months without significantly impacting its ability to preserve microorganisms (e.g., anaerobic bacteria) of a biological sample (e.g., a stool sample) for at least about 1, 2, 3, 4, 5, 6, 7, 8, or more days. A formulation that has been stored for several months can have an ability to preserve a microorganism (e.g., an anaerobic bacterium) that is at least about 50%, 60%, 70%, 80%, 85%, 90%, 95%, 97%, or 99% or equal to the ability to preserve a microorganism (e.g., an anaerobic bacterium) of a freshly produced formulation consisting of the same or different components. Freshly produced formulations herein can include those that are at most 10, 9, 8, 7, 6, 5, 4, 3, 2, or 1 day old, or less.

In some cases herein, any one of the formulations shown in TABLES 1-4 can be stored for at least 5 months without impacting the formulation's capacity to preserve anaerobic bacteria of a stool sample for at least about 5 days, wherein the preservation capacity of the formulation stored for 5 months is at least about 80% of the preservation capacity of a freshly prepared formulation.

### Methods of Use

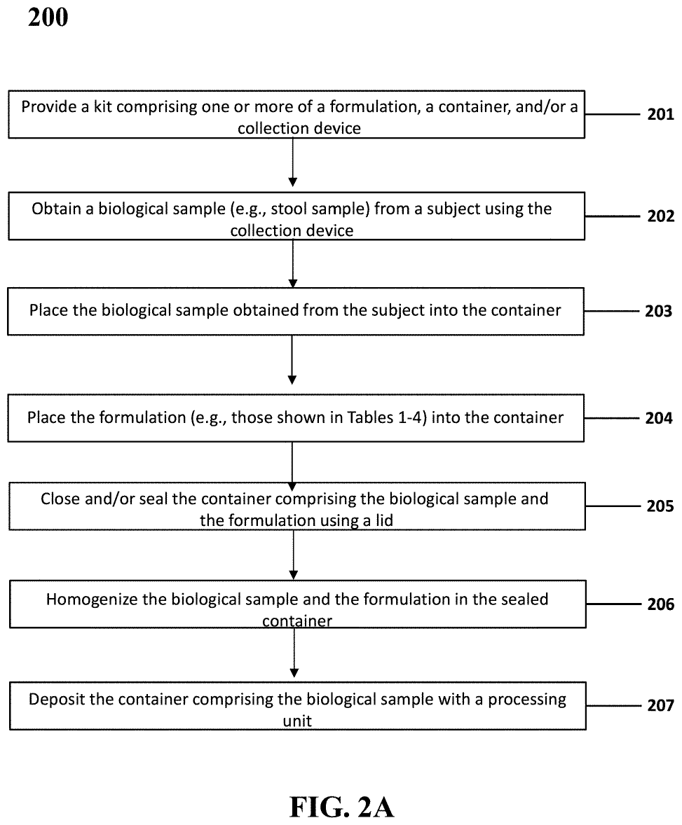

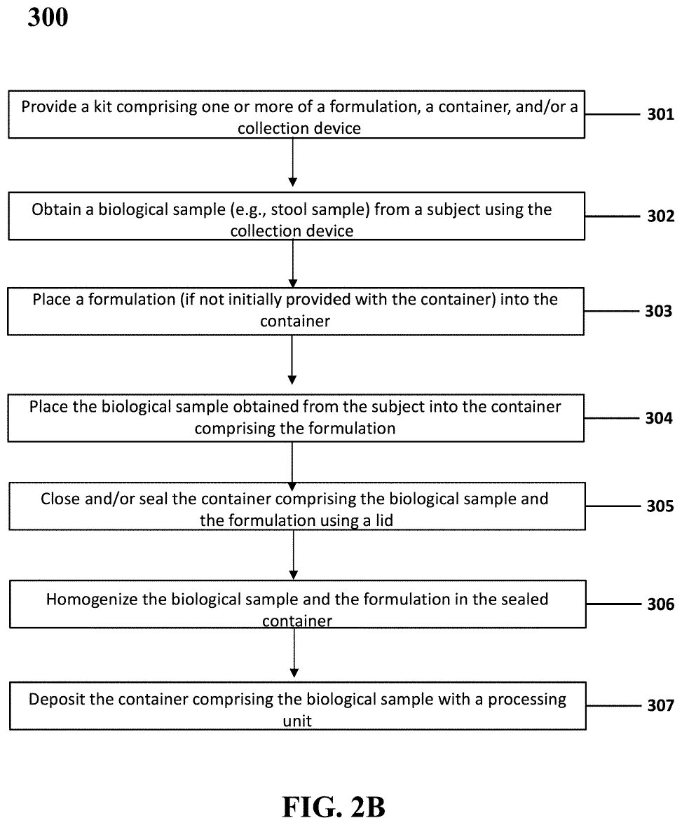

The present disclosure also provides methods for obtaining a stool sample comprising at least one anaerobic microorganism from a subject and preserving the at least one anaerobic microorganism. FIGS. 2A and 2B show an example methods 200 and 300 for obtaining a stool sample comprising at least one anaerobic microorganism from a subject, and preserving the at least one anaerobic microorganism during sample collection, transport, storage, and/or processing and analysis.

For example, in operation 201, a kit comprising one or more of a formulation, a container and/or a collection device can be provided. Next, in operation 202, the collection device (e.g., comprising a scoop) can be used to obtain a stool sample from the subject (e.g., a human). Subsequently, in operation 203, the stool sample obtained from the subject can be placed and/or stored in a container comprising a formulation. The formulation can comprise at least one antioxidant, at least one oxygen scavenger and at least one metabolite scavenger. In some cases (e.g., if not initially provided with the container), in operation 204 the formulation can be placed into the container. Next, in operation 205, the container comprising the stool sample and the formulation can be closed and/or sealed using a lid, such that a concentration of oxygen gas in a gaseous space or gaseous medium in the container, subsequent to closing and/or sealing, can be less than about 20.95% for a time period of at least about 2 days to at least about 7 days as measured at 25 degree Celsius (° C.). Next, in operation 206, the stool sample and the formulation can be homogenized in the sealed container. Subsequently, in operation 207, the container comprising the stool sample can be deposited with a processing unit that may process and/or analyze the stool sample or derivative thereof.

The anaerobic collection formulations provided herein can preserve microbial species of a biological sample for at least about 1, 2, 3, 4, 5, or 6 days following collection of the sample and transfer of the sample to an anaerobic collection formulation in a collection tube. The collection formulations herein can preserve the microbial species (e.g., bacterial species) of a biological sample at room temperature (e.g., a temperature from about 32° C. to about 37° C.) or at lower temperatures, e.g., from about 4° C. to about 0° C., or colder temperatures down to about −80° C. In various instances herein, the anaerobic collection formulation can be a formulation that comprises or consists of the components listed in any one of TABLES 1-4. In various instances herein, such collection formulation can preserve the viability and/or abundance of microorganisms of a biological sample (e.g., a stool sample) for at least about 1, 2, 3, 4, 5, or 6 days following collection of the sample. The preservation of viable microorganisms in a collection formulation can be determined by culturing the microorganisms after storage for a certain period of time (e.g., 24, 48, 72, 96, or 120 hours) at a certain temperature (e.g., at room temperature or at −80° C.). The microorganisms can be cultured using various media, such as BHI, CHOC, YCFAC+B, high-dilution media, etc. Following culture, the preserved and cultured microorganisms can be identified and quantified using various techniques, including whole-genome sequencing (WGS), such as shot-gun sequencing. The abundance of the various microorganism that were cultured from the stored samples can be determined and compared to the abundance of such microorganisms cultured from a fresh biological sample. The results obtained from such experiments can be displayed as a percent (%) relative abundance as a function of storage and culture conditions, wherein the abundance of a microorganism of a stored sample is relative to the abundance of the microorganism in a fresh (not stored) sample.

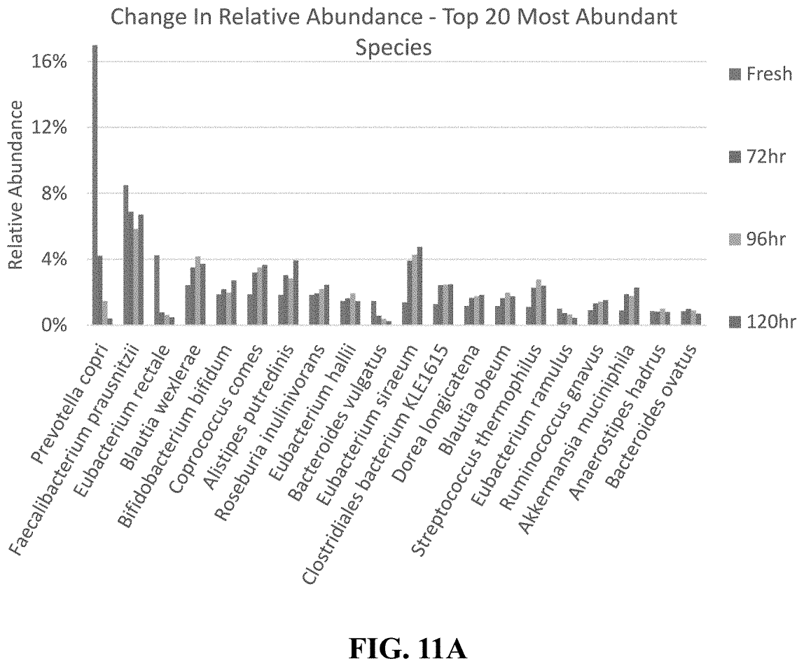

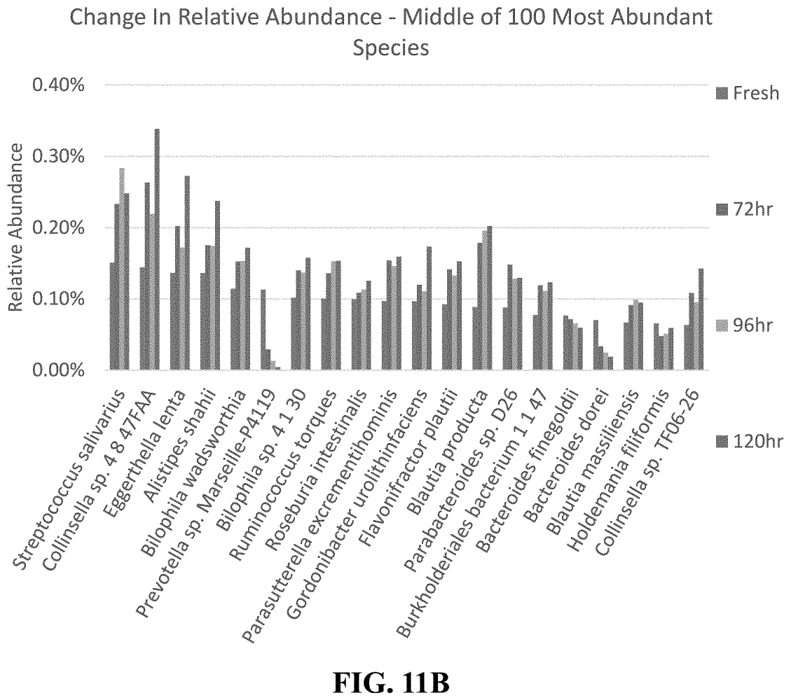

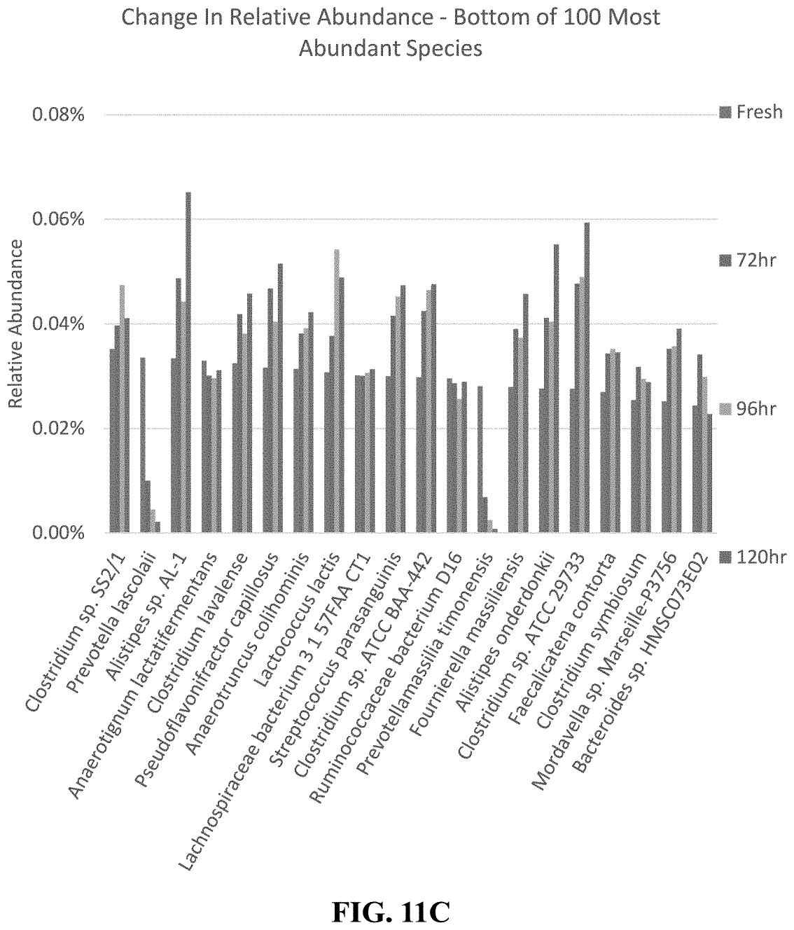

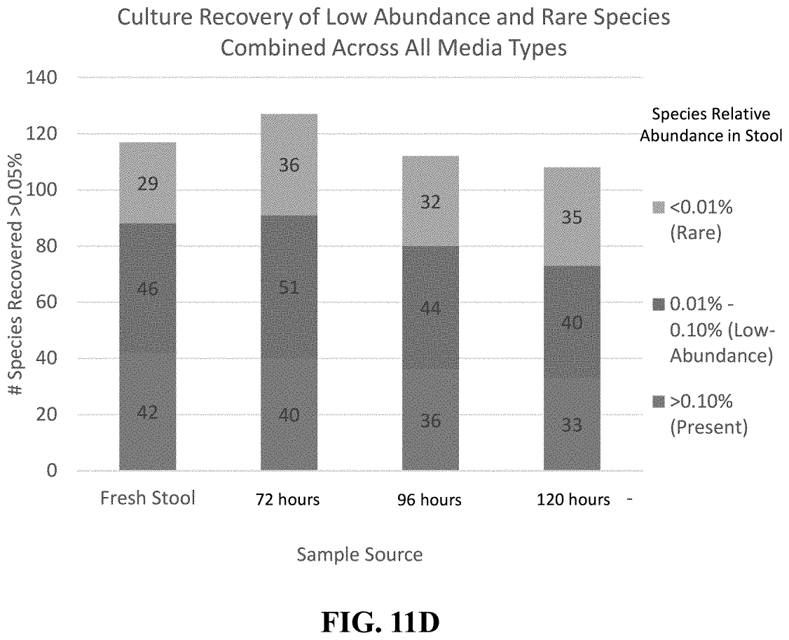

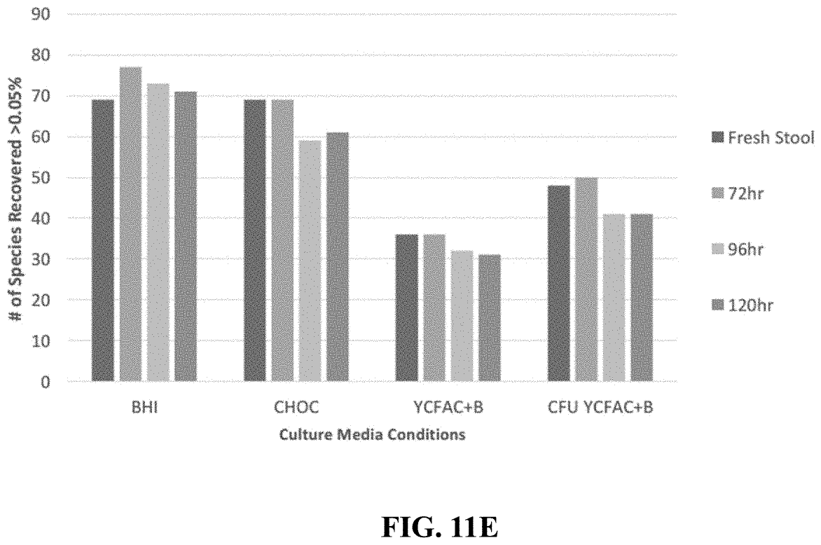

The methods provided herein allow preservation of at least about 5%, 10%, 15%, 20%, 25%, 35%, 45%, 50%, 75%, or 85% of the 25 most abundant species in a biological sample. In some instances, such methods can also preserve at least about 5%, 10%, 15%, 20%, 25%, 35%, 45%, 50%, 75%, or 85% of the middle 20 of the 100 (i.e., 40-60) most abundant species in the biological sample. In some instances, such methods can also preserve at least about 5%, 10%, 15%, 20%, 25%, 35%, 45%, 50%, 75%, or 85% of the bottom 20 of the 100 (i.e., 80-100) most abundant species in the biological sample. In various instances herein, preservation of a microorganism (e.g., a bacterium, fungus, etc.) can be defined as preserving at least about 70%, 75%, 80%, 85%, 90%, 95%, 97%, 99%, or 100% of the abundance of the microorganism for a given period of storage time (e.g., 1, 2, 3, 4, 5, or more days) relative to a fresh biological sample. In such instances, preservation of a microorganism can be determined by comparing the quantity of such microorganism obtained from culture after storage in the collection formulation compared to the quantity of such microorganism obtained from culture of a fresh sample. As an example, FIG. 11A shows a change in relative abundance of the 20 most abundant species in a fresh stool sample compared to a stool sample that was stored in an aerobic collection formulation comprising the components shown in TABLE 4 for 72, 96, or 120 hours at room temperature, respectively. These data show that, e.g., the viability of Faecalibacterium prausnitzii and Akkermansia muciniphila can be preserved during storage for at least 5 consecutive days, enabling successful cell culture that demonstrated that at least 70% abundance of this microorganism was preserved.

The methods provided herein allow preservation of viability of at least one microorganism in a formulation at an abundance of least about 50%, 60%, 70%, 80%, 90%, or 95% of the at least one microorganism as compared to an abundance of the at least one microorganism in a fresh biological sample. Such viability of at least about 50% can be preserved when the at least one microorganism is stored in the formulation for a time period of at least 1, 2, 3, 4, 5, 6, or more days (e.g., consecutive days). Further provided herein are methods that can preserve a viability of at least one microorganism in a formulation at an abundance of least about 50%, 60%, 70%, 80%, 90%, or 95% of the at least one microorganism as compared to an abundance of the at least one microorganism in a fresh biological sample, wherein such at least one microorganism has a relative abundance in the biological sample of at least about 0.0001%, 0.001%, 0.01%, or 0.1%. In other instances, such relative abundance of the at least one microorganism in the biological sample can be less than about 0.1%, 0.01%, 0.001%, or 0.0001%. In some instances, such viability can be preserved for at least 1, 2, 3, 4, 5, 6, 7, 8, 9, 10, 15, 20, 25, 50 or more microorganisms. Such microorganisms can be bacteria. In some cases, at least one of such bacteria is an anaerobic bacterium. In other cases, all of such bacteria are anaerobic bacteria.

Sample Collection. Sample collection procedures including the herein disclosed kits, methods, and systems can vary depending on the intended use. In order to preserve the anaerobic environment inside the sample tube or container, the packaging (e.g., foil packaging) may be removed from the tube or container, opened, and the tube brought to where the sample can be produced and/or collected, immediately prior to sample generation.

The biological sample can be provided by a subject (e.g., a human). The subject can be a human. The biological sample can be a stool sample. The human subject providing the stool sample can place an object such as a paper spread across a toilet or plastic contain to catch their stool, without the sample tube or containing touching the inside of the toilet. The human subject can open the tube or container and add a certain amount of a stool sample into the sample tube or container comprising a microbial anaerobic collection and preservation formulation (e.g., those comprising one or more of the components shown in any one of TABLES 1-4). The sample tube or container can comprise a collection device such as a scoop or brush. The collection device can allow the addition of a certain amount of stool sample to the formulation (e.g., a formulation for transport and/or storage). This can achieve a specific concentration or concentration range of the sample in the formulation, which can be used for subsequent processing and/or analysis steps (e.g., sequencing, cell culturing, etc.).

A sample (e.g., a stool sample) may be collected and transferred directly into an anaerobic sample collection device of this disclosure using a collection device as described herein (e.g., scoop, swabs, spatula, etc.). Alternatively, a sample may be initially collected using a bucket or bucket-style vessel as an initial collection device. Such an initial collection device may allow larger amounts of volumes of sample to be collected. One or more fractions of such an initially collected sample may be placed in a sample collection formulation and/or container as described herein. The initially collected sample may be homogenized prior to transfer of a sample fraction into an anaerobic sample collection device of this disclosure.

Advantages of the kits, methods, and systems of the present disclosure include the ability to not only process or analyze a biological sample, but also to culture and grow one or more microbial species obtained from the biological sample. This may allow in-depth studies and analyses of the microorganisms of a biological sample. In cases where the biological sample is obtained from an animal (e.g., a human), the herein disclosed methods can allow for development of animal models and/or live biotherapeutic products such as fecal transplants or bacterial composition comprising one or more purified and isolated microorganisms (e.g., bacteria) for preventative and/or therapeutic purposes.

Laboratory Processing. A sterile sample kit as described herein can comprise a formulation comprising a biological sample can be delivered to a processing and/or analysis laboratory or facility within hours or days of sample collection. As described herein, the kits, methods, and systems of the present disclosure can maintain the viability of microorganisms for at least about 6 days, at least about 7 days, or at least about 10 days. This may allow sample collection at various sample collection sites and may enable the use of large cohort sizes and diverse samples for studies and analyses (e.g., for large and diverse microbiome studies).

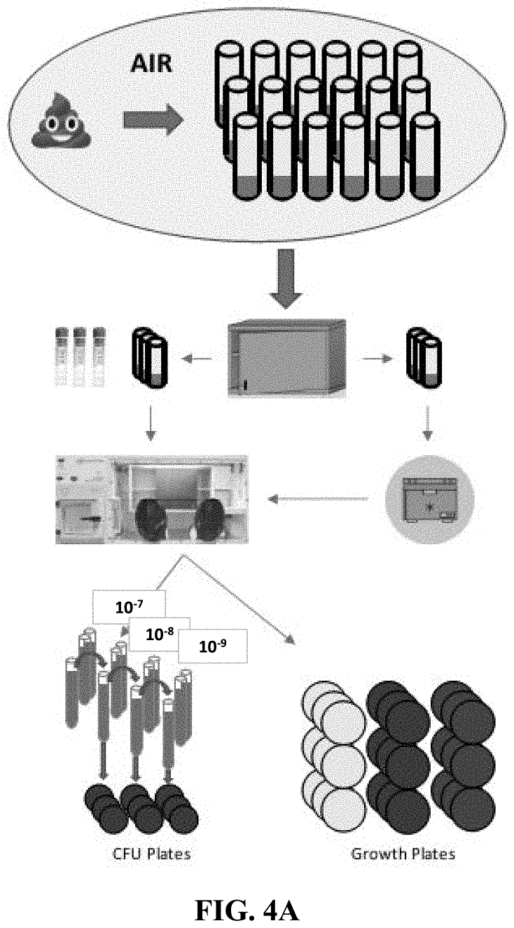

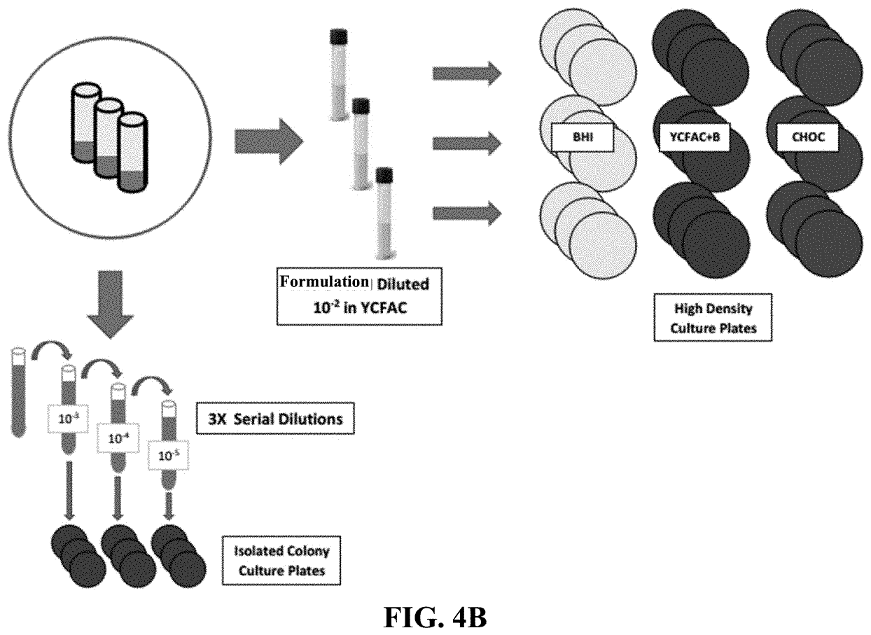

Once the sample arrives at the processing facility, the formulation comprising the biological sample can be homogenized through, e.g., vortexing in a laboratory and/or using one or more homogenizer(s) during sample collection, transport, and/or storage. Homogenization can simplify transfer of sample, e.g., by liquifying the sample and allowing pipetting of specific volumes. The sample can then either immediately be placed in a −80° C. freezer or further processed, e.g., inside of an anaerobic chamber (e.g., FIG. 4A). Once opened in an anaerobic chamber, the sample may still be frozen at about −80° C. A container comprising a formulation comprising a sample that is frozen may remain frozen until further use, and a fraction of the sample can be taken by scraping a portion of the still frozen contents off the tube before placing the tube back into the freezer. In some cases, for sample pre-processing, a biological samples such as a stool sample can be combined with a specified volume of buffer or other dilution medium inside an anaerobic chamber (e.g., to achieve a certain sample to formulation weight ratios of 1:1, 1:2, 1:3, etc.). Such medium can be pre-chilled (e.g., to 4° C.) pre-reduced anaerobically sterilized (PRAS) dilution blank medium. Such medium can be supplemented with 20% glycerol. For further sample processing, serial dilutions using a culture medium can be conducted to yield a series sample dilutions. In some cases, such culture medium can be PRAS yeast casitone fatty acid with carbohydrate (YCFAC) broth.

Sample Storage. The kits, methods, and systems of the present disclosure can be used to store a biological sample. During storage, the microorganisms present in the biological sample can remain viable. A biological sample can be stored for various time periods. Such time periods can range from minutes to several months, which can depend on use and sample composition. Using the kits, methods, devices and compositions described herein, a biological sample can be stored for at least about 1 hour, at least about 10 hours, at least about 24 hours, at least about 48 hours, or at least about 72 hours. Using the kits, methods, and compositions described herein, a biological sample can be stored for at least about 1 day, at least about 2 days, at least about 3 days, at least about 4 days, at least about 5 days, at least about 6 days, at least about 7 days, at least about 10 days, or at least about 20 days. Using the kits, methods, and compositions described herein, a biological sample can be stored for at least about 1 week, at least about 2 weeks, at least about 3 weeks, at least about 4 weeks, at least about 5 weeks, at least about 6 weeks, at least about 7 weeks, at least about 10 weeks, or at least about 20 weeks. Using the kits, methods, and compositions described herein, a biological sample can be stored for at least about 1 month, at least about 2 months, at least about 3 months, at least about 4 months, at least about 5 months, at least about 6 months, at least about 7 months, at least about 10 months, or at least about 20 months. Using the kits, methods, and compositions described herein, a biological sample can be stored for at least about 1 year, at least about 2 years, at least about 3 years, at least about 4 years, at least about 5 years, at least about 6 years, at least 7 about years, at least about 10 years, or at least about 20 years.

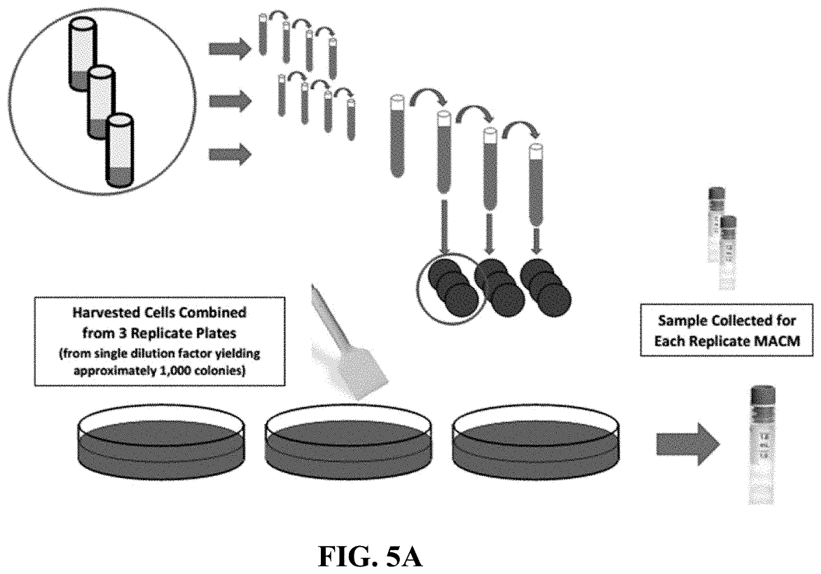

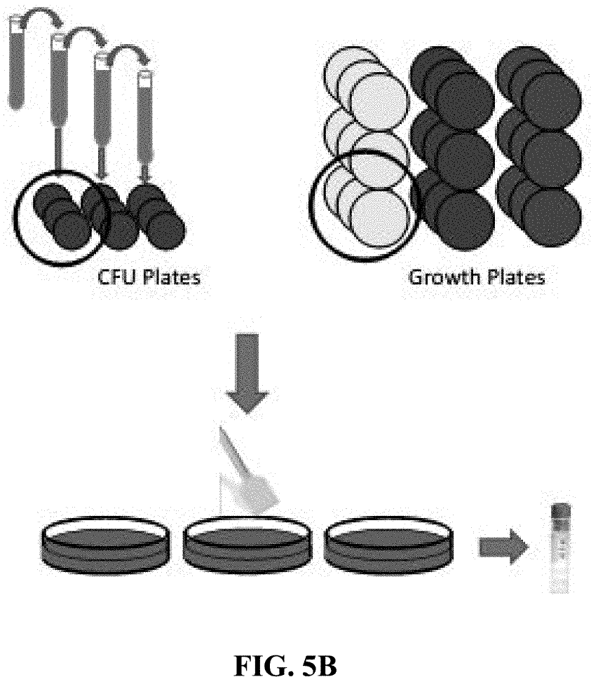

Other Uses for Collection Formulations. The microbiota anaerobic collection formulations of the present disclosure can be used for various applications. Such applications can include sample collection, transport, analysis and processing, cell culture, generation of culture media for various microorganisms (e.g., animal and non-animal culture media, soy broth, tryptic soy broth). For example, the kits, methods, and compositions described herein can be used to culture anaerobic microorganisms such as strict and/or facultative anaerobic bacteria. Culturing of microorganisms (e.g., bacterial species) after storage can be conducted using various media, such as BHI, CHOC, YCFAC+B, high-dilution media (e.g., using YCFAC+B media), etc. (e.g., FIG. 4A-FIG. 5B). Following culture, the preserved and cultured microorganisms can be identified and quantified using various techniques, including whole-genome sequencing (WGS), such as shot-gun sequencing.

### EXAMPLES

The following examples merely illustrate the disclosure and are not intended to limit the disclosure in any way.

### Example 1

### Collection Formulation for Anaerobic Bacteria Isolated from a Stool Sample

This example demonstrates a microbiota anaerobic collection formulation and container for obtaining a biological sample from a subject, wherein the biological sample can comprise one or more anaerobic bacterial species (e.g., OTUs). The herein described collection formulation can be used to collect a stool sample from a subject, wherein the stool sample can comprise one or more anaerobic bacterial species.

The collection formulation described in this example comprises several ingredients and components, each of which has a primary use, but can have multiple uses, depending on the collection formulation and its intended use. Examples of components and ingredients of a collection formulation used in this example and this disclosure are shown in TABLES 1-4.

The collection formulations of the present example comprise sodium thioglycolate and L-cysteine as reducing agents (e.g., oxygen scavengers) that help maintain anaerobic conditions in the formulation, and the oxygen scavenger glutathione and peroxidase were used to protect against cellular damage. Glutathione can protect cysteine residues from oxidation, and peroxidase can metabolize peroxides. Mineral salts such as sodium phosphate, sodium chloride, potassium chloride, and potassium phosphate were used to maintain the pH of the formulation, as well as the osmotic, and electrolytic balance for cell preservation. Ascorbic acid was used to scavenge oxygen radicals. Catalase provided decomposition of hydrogen peroxide that may have been produced by the microorganisms during storage and/or transport. In some cases, a collection formulation can comprise activated carbon powder to adsorb and neutralize short carbon-chain fatty acid byproducts from the organisms which can inhibit growth as shown in, e.g., a formulation for preserving microorganisms described in TABLE 3.

TABLES 1-4 below show components of exemplary formulations for collection, storage and preservation of diverse microorganisms, including anaerobic microorganisms, present in a biological sample (e.g., a human stool sample).

TABLE 1
Components of an Exemplary Sample Collection Formulation

| | Amount | | |
|---|---|---|---|
| Substance | (per 1 liter (L) of formulation) | | Use |
| Sodium Thioglycolate | 1 | g | Oxygen Scavenger |
| Sodium Phosphate Dibasic | 1.15 | g | Electrolyte |
| Sodium Chloride | 3 | g | Electrolyte |
| Potassium Chloride | 0.2 | g | Electrolyte |
| Potassium Phosphate Monobasic | 0.2 | g | Electrolyte |
| Magnesium Sulfate Heptahydrate | 0.1 | g | Electrolyte |
| L-Cysteine | 1 | g | Oxygen Scavenger |
| Glycerol | 200 | mL | Cryoprotectant |
| Activated Carbon* | 1 | g | Metabolite Scavenger |
| Antioxidant Enzymes | 0.1-1 g and/or | | Oxygen Scavengers/ |
| | 10 U-500,000 U | | antioxidants |
| DI Water | 800 | mL | Solvent |

TABLE 2
Components of an Exemplary Sample Collection Formulation

| | Amount (per 1 L | | |
|---|---|---|---|
| Substance | of formulation) | | Use |
| Sodium Thioglycolate | 1 | g | Oxygen Scavenger |
| Sodium Phosphate Dibasic | 1.15 | g | Electrolyte |
| Sodium Chloride | 3 | g | Electrolyte |
| Potassium Chloride | 0.2 | g | Electrolyte |
| Potassium Phosphate Monobasic | 0.2 | g | Electrolyte |
| Magnesium Sulfate Heptahydrate | 0.1 | g | Electrolyte |
| L-Cysteine | 1 | g | Oxygen Scavenger |
| Glycerol | 200 | mL | Cryoprotectant |
| Activated Carbon Powder | 0.2 | g | Metabolite Scavenger |
| L-Ascorbic Acid | 0.2 | g | Antioxidant |
| Antioxidant Enzymes | 0.1-1 g and/or | | Oxygen Scavengers/ |
| | 10 U-500,000 U | | antioxidants |
| DL-Dithiothreitol | 0.15 | g | Antioxidant/ |
| | | | Preservative for |
| | | | Enzymes |
| DI Water | 800 | mL | Solvent |

TABLE 3
Components of an Exemplary Collection Formulation

| | Amount (per 1 L | | |
|---|---|---|---|
| Substance | of formulation) | | Use |
| Sodium Thioglycolate | 1 | g | Oxygen Scavenger |
| Sodium Phosphate Dibasic | 1.15 | g | Electrolyte |
| Sodium Chloride | 3 | g | Electrolyte |
| Potassium Chloride | 0.2 | g | Electrolyte |
| Potassium Phosphate Monobasic | 0.2 | g | Electrolyte |
| Magnesium Sulfate Heptahydrate | 0.1 | g | Electrolyte |
| L-Cysteine | 1 | g | Oxygen Scavenger |
| Glycerol | 200 | mL | Cryoprotectant |
| Activated Carbon Powder | 0.2 | g | Metabolite Scavenger |
| L-Ascorbic Acid | 0.2 | g | Antioxidant |
| Glutathione | 0.4 | g | Oxygen Scavenger/ |
| | | | antioxidant |
| Glutathione Peroxidase | 20 | Units | Oxygen Scavenger/ |
| | | | antioxidant |
| Peroxidase | 500 | Units | Oxygen Scavenger/ |
| | | | antioxidant |
| Superoxide Dismutase | 5,000 | Units | Oxygen Scavenger/ |
| | | | antioxidant |
| Catalase | 140,000 | Units | Oxygen Scavenger/ |
| | | | antioxidant |
| DL-Dithiothreitol | 0.15 | g | Antioxidant/ |
| | | | Preservative for |
| | | | Enzymes |
| DI Water | 800 | mL | Solvent |

TABLE 4
Components of an Exemplary Collection Formulation

| | Amount | |
|---|---|---|
| | (per 1 L of | |
| Substance | formulation) | Use |
| Sodium Thioglycolate | 1 g | Oxygen Scavenger |
| Sodium Phosphate Dibasic | 1.15 g | Electrolyte |
| Sodium Chloride | 3 g | Electrolyte |
| Potassium Chloride | 0.2 g | Electrolyte |
| Potassium Phosphate Monobasic | 0.2 g | Electrolyte |
| Magnesium Sulfate Heptahydrate | 0.1 g | Electrolyte |
| L-Cysteine | 1 g | Oxygen Scavenger |
| Glycerol | 200 mL | Cryoprotectant |
| L-Ascorbic Acid | 0.2 g | Antioxidant |
| Glutathione | 0.4 g | Oxygen Scavenger/ |
| | | antioxidant |
| Glutathione Peroxidase | 20 Units | Oxygen Scavenger/ |
| | | antioxidant |
| Peroxidase | 500 Units | Oxygen Scavenger/ |
| | | antioxidant |
| Superoxide Dismutase | 5,000 Units | Oxygen Scavenger/ |
| | | antioxidant |
| Catalase | 140,000 Units | Oxygen Scavenger/ |
| | | antioxidant |
| DL-Dithiothreitol | 0.15 g | Antioxidant/ |
| | | Preservative for |
| | | Enzymes |
| DI Water | 800 mL | Solvent |

The collection and storage formulations shown in TABLES 1-4 were produced using the ingredients listed therein. A biological sample was then added to an anaerobic collection tube containing a certain volume of such formulation, e.g., about 10 mL, 12 mL, 14 mL, or 20 mL.

An exemplary sterile formulation contained a buffered mineral salt solution and enzymes. The formulation was non-nutritive to minimize overgrowth during transport. Formulation vials were dispensed and packaged under strict anaerobic conditions. The final solution had a pH of about 7.5, a fill volume of about 9.0 milliliter (mL) in a 12.0 mL tube.

An exemplary sterile container was an opaque polypropylene tube having a 16 mm diameter and about 6 cm or 10 cm tall, with a screw cap and with or without an integrated scoop in the cap. In some instances, a container can have a 16 mm diameter and be 10 cm tall, with a screw cap not including an integrated scoop. This container, comprising 9 ml of formulation and a 3 ml empty headspace, was configured to add 1 g of a biological sample to the container, resulting in an approximate 1:10 dilution of the sample. This approach minimized oxygen in the headspace, while allowing for expansion of the liquid when freezing without damaging the tube.

In another example, 14 mL of a formulation (e.g., the formulation of TABLE 4) was loaded into a 101×16.5 mm plastic tube or translucent polypropylene tube with scoop (Sarstedt, part number 80.623.022) under anaerobic conditions and then placed in a foil pouch under anaerobic conditions. Such plastic tube and foil pouch are shown in FIG. 1.

### Example 2

### Formulation Preserves Microbial Organisms of a Stool Sample During Transport

This example demonstrates that a herein described microbiota anaerobic collection formulation and container preserved the viability of microbial organisms of a stool sample obtained from a subject during transport for at least 96 hours after sample collection.

The kit used in this example comprised a formulation comprising the components listed in TABLE 1. The formulation was non-nutritive to minimize overgrowth during transport. Formulation vials were dispensed and packaged under strict anaerobic conditions. The final solution had a pH of about 7.5 and a fill volume of about 9.0 mL in a 12.0 mL tube. The sample collection container used in this example was an opaque polypropylene tube having a 16 mm diameter and about 10 cm tall, with a screw cap without an integrated scoop. This container, comprising a 9 ml of formulation and a 3 ml empty headspace, was configured to add a 1 g sample of a biological sample that was placed in the container, resulting in an approximate 1:10 dilution of the sample. This approach minimized oxygen in the headspace, while allowing for expansion of the liquid when freezing without damaging the tube.

The stool sample was obtained from a human subject using a bucket container, homogenized, brought into air to simulate sample collection by a subject, and then 1 g of stool sample was added to the container, in air, using a pipette. The container was closed after the stool sample was added to the transport formulation. The container was shaken or vortexed to homogenize the suspension of stool matter in the collection formulation. The viability of the microbial composition of the stool sample was maintained during transport. The bacteria obtained from the biological sample were cultured under anaerobic conditions and analyzed using whole-genome shotgun sequencing (WGS), demonstrating that the transport formulation preserved the viability of the microorganisms (e.g., bacteria) of the stool sample and allowed in-depth analysis of the microbial composition.

This example demonstrates that collection of biological samples comprising anaerobic bacteria using the herein disclosed kits, methods, and systems preserves the viability of the bacteria during transport.

### Example 3

### Sample Collection, Transport, and Further Processing Using a Formulation

This example demonstrates sample collection using a herein described microbiota anaerobic collection formulation and container to preserve the viability of microbial organisms of such sample. The sample was a stool sample obtained from a human subject.

The stool sample was obtained from a human subject using a bucket container, homogenized, brought into air to simulate sample collection by a subject, and then 1 g of stool sample was added to the container, in air, using a pipette. The container was filled with an anaerobic formulation and an anaerobic atmosphere. The anaerobic formulation used in this example comprised the components listed above in TABLE 1.

The microorganisms present in the stool sample were preserved and their viability was maintainer after sample collection and during transport to a processing and analysis facility. After a transport and/or storage period of at least 72-120 hours, the sample tube or container comprising the collected human stool sample was placed into an anaerobic chamber (e.g., a glove box) for further processing including cell culture and quantitative as well as qualitative analysis using sequencing.

Analysis of the microorganisms present in the stool sample showed that over 80% of the microorganisms remained viable during the collection, transport, and/or storage period. The microorganisms of the sample were sequenced to analyze the microbial composition of the stool sample. Such analysis provides information about the subject's intestinal microbiome composition and may have associated health implications.

Parallel to, or in addition to sequencing, the microorganisms of the collected stool sample were placed into culture media for further growth of the microbial communities present in the stool sample (FIG. 4A-FIG. 5B). Such microbial species (e.g., bacteria beneficial to human and/or non-human animal health) can be further used to assemble a live biotherapeutic microbial consortium. Such live biotherapeutic microbial consortia can be used to prevent and/or treat a disease or condition (e.g., a dysbiosis) in a subject, wherein the subject can be the subject that provided the sample and/or a different human or non-human subject.

This example demonstrates that the herein disclosed kits, methods, and systems can allow for collection of biological samples, particularly samples that comprise one or more anaerobic microorganisms, and that such kits, methods, and systems provide conditions that maintain the viability of such microorganisms during and after sample collection, transport, storage, and further processing and analysis.

### Example 4

### Anaerobic Sampling and Culture Media

This example demonstrates anaerobic sampling and culturing of strict anaerobic and facultative anaerobic microorganisms isolated from a biological sample that was collected using the herein described kits, methods, and systems.

A biological sample collected using the kits, methods, and systems described herein was placed into an anaerobic chamber upon arrival at a processing and/or analysis facility (e.g., a hospital, a research laboratory, etc.) after sample collection, transport and/or storage (FIG. 4A).

The following media were chosen for the cultivation of strictly anaerobic and facultatively anaerobic microbes of a biological sample (the recipes are given for 1 liter medium to be prepared with deionized water (as referred to herein as “DI water”): TG (thioglycolate liquid medium) (peptone from casein (Becton Dickinson [BD], NJ), 15.0 gram (g); yeast extract (BD), 5.0 g; D-(+)-glucose, 5.5 g; NaCl, 2.5 g; sodium acetate, 3.0 g; cysteine-HCl, 0.5 g; sodium thioglycolate, 0.5 g; sodium resazurin, 0.001 g; gas phase, N₂; pH 7.1); TGA (thioglycolate agar plates) (TG medium plus agar, 15.0 g); TS (trypticase soy liquid medium) (TS broth [BD], 30.0 g; sodium resazurin, 0.001 g; sodium thioglycolate, 0.5 g; cysteine-HCl, 1.0 g; gas phase, N₂ [80%] and CO_{2 [20}%]); TSA (trypticase soy agar plates) (TS medium plus agar, 15.0 g); SRA (sulfate reducer agar plates, medium based on DSMZ medium no. 63, modified) [KH₂PO₄, 0.47 g; NH₄Cl, 1.0 g; CaCl₂:2H₂O, 0.1 g; yeast extract (BD), 1.0 g; Na₂SO₄, 1.0 g; MgSO₄.7H₂O, 2.0 g; (40%) (wt/vol) L-(+)-lactate, 2.5 ml; FeSO₄.7H₂O, 0.004 g; agar, 10.0 g; sodium resazurin, 0.001 g; ascorbic acid, 0.2 g; sodium thioglycolate, 0.2 g; pH 7.0]; MM (methanogenic Archaea liquid medium) (NH₄Cl, 0.5 g; KH₂PO₄, 0.4 g; MgCl₂.6H₂O, 0.15 g; CaCl₂.2H₂O, 0.05 g; trace element solution [10×], 1 milliliter (ml); vitamin solution [10×], 1 ml; sodium resazurin, 0.001 g; Na₂S, 0.5 g; cysteine-HCl, 0.5 g; gas phase, H₂ [80%] and CO₂[20%]); BM (basal medium) (NH₄Cl, 0.5 g; KH₂PO₄, 0.4 g; MgCl₂.6H₂O, 0.15 g; CaCl₂.2H₂O, 0.05 g; NaHCO₃, 1.0 g; trace element solution [10×], 1 ml; vitamin solution [10×], 1 ml; sodium resazurin, 0.001 g; Na₂S, 0.25 g; cysteine-HCl, 0.25 g; pH 7.0); ASM (Archaea-supporting liquid medium) (per 20 ml BM, add 0.1% [wt/vol] sterile yeast extract prior to inoculation; add 0.1 ml of an antibiotic mixture [carbenicillin (0.2% [wt/vol]), streptomycin (0.2% [wt/vol]), rifampin (0.4% [wt/vol]), and cephalosporin (0.2% [wt/vol])]; gas phase, N₂; pH 7.0); AHM (autotrophic homoacetogen liquid medium) (per 20 ml BM, add 0.2 ml 2-bromoethanesulfonic acid [2 M]; gas phase, H₂ [80%] and CO₂ [20%]); N₂ fix (Hino and Wilson N₂-free liquid medium, sucrose, 20.0 g; MgSO₄.7H₂O, 0.5 g; NaCl, 0.01 g; FeSO₄.7H₂O, 0.015 g; Na₂MoO₄.2H₂O, 0.005 g; CaCO₃, 10.0 g; solve ingredients in 1 liter of K₂HPO₄—KH₂PO₄ buffer [0.1 M, pH 7.7]; medium was not reduced; after sterilization, add 5 μg biotin and 10 μg 4-p-aminobenzoic acid per liter; gas phase, N₂); AAM (autotrophic all-rounder liquid medium; KH₂PO₄, 0.4 g; CaCl₂.2H₂O, 0.05 g; MgCl₂.6H₂O, 0.15 g; NaHCO₃, 1.5 g; Fe₂O₃.9H₂O, 0.25 g; NaNO₃, 0.5 g; Na₂S₂O₃.5H₂O, 1.56 g; trace element solution [10×], 1 ml; vitamin solution [10×], 1 ml; sodium resazurin, 0.001 g; Na₂S, 0.5 g; gas phase, N₂ [80%] and CO₂ [20%]).

Any such culture medium can be used to further grow microorganisms (e.g., of a human stool sample) that are stored in a formulation and that were kept viable during sample collection, transport, and/or storage, which can be at least 50%, 60%, 70%, 80%, 90%, or at least 95% of microbial species present in a given biological sample. These microbial populations may provide valuable information into a subject's microbiome such as the intestinal microbiome and may allow for further diagnostic and/or therapeutic interventions (e.g., against dysbiosis, infections, etc.), which demonstrates a significant advantage over conventional methodologies for collecting and transporting biological samples.

### Example 5

### Evaluation of Microbiota Specimen Collection Kit and Formulation

This example shows data evaluating a microbiome collection kit comprising a formulation that can preserve live microbiota samples for the purpose of growth, analysis and isolation of microorganisms.

Overall, the data described in this EXAMPLE demonstrate that the collection formulation (composition as shown in TABLE 4) and device effectively preserved microbiota at room temperature for at least 72 to 120 hours without substantial loss of viability. The data further showed that samples collected with such a collection device can also be directly frozen in collection kits for long term storage (e.g., at −80° C.).

### Background

The collection and culture of organisms from complex biological samples, such as feces or stool, can be challenging. Obligate anaerobic organisms contained in the samples can be killed in the presence of atmospheric oxygen. Acidic and other metabolic byproducts can kill microorganisms in the sample if growth continues after collection. The growth and proliferation of some fast-growing organisms in a sample can prevent the recovery of other low relative abundance, fastidious, or slow growing organisms. Current collection techniques for culturing samples are not practical or cost effective. As an example, fresh samples require the participant to provide the sample at the lab. Alternatively, samples collected at home can be frozen and cold-shipped, but many microorganisms cannot be cultured after freeze-thawing without a cryoprotectant. The microbiome collection device was developed in response to a need for a practical, low cost, and user-friendly system of collecting, transporting, and preserving microbiota samples for the purpose of culturing the microorganisms within the sample.

### Collection and Microbial Preservation Device, Composition, and Usage Information

The sample collection device used in this EXAMPLE comprised a 16 mm×100 mm stool collection tube without an integrated collection scoop, using a pipette for transferring a 1 sample. However, a similar tube with a cap having an integrated scoop can alternatively be used. The collection formulation used in this study was consisted of the components listed in TABLE 4.

### Sample Collection

The stool sample was obtained from a human subject using a bucket container, homogenized, and brought into air to simulate sample collection by a subject. The outer foil packaging was then removed from the collection tube comprising the collection formulation and the cap from the collection tube was removed. Using a pipette, 1 g of stool sample was added to the container, in air. The tube was sealed and vigorously shaken for about 30 seconds to homogenize the sample.

The collection device comprising the formulation and the sample was stored at room temperature or refrigerated. Short-term exposure to higher temperatures, such as during shipping or mailing, did not affect product performance. The shelf-life of the device and collection formulation was about 6 months. Microbiota samples, when collected prior to expiration of the shelf-life, can be preserved and stored at −80° C. for extended periods beyond expiration. Collection tubes were individually packaged in aluminum foil pouches under anaerobic conditions.

### Example 6

### Sample Collection and Processing

This example demonstrates the preservation of viable anaerobic microorganisms stored for up to 120 hours at 25° C. in the formulation with or without subsequent storage at −80° C. for an additional time period (e.g., 7 consecutive days). The biological sample used in this EXAMPLE was a human stool sample.

### Stool Collection and Pre-Processing

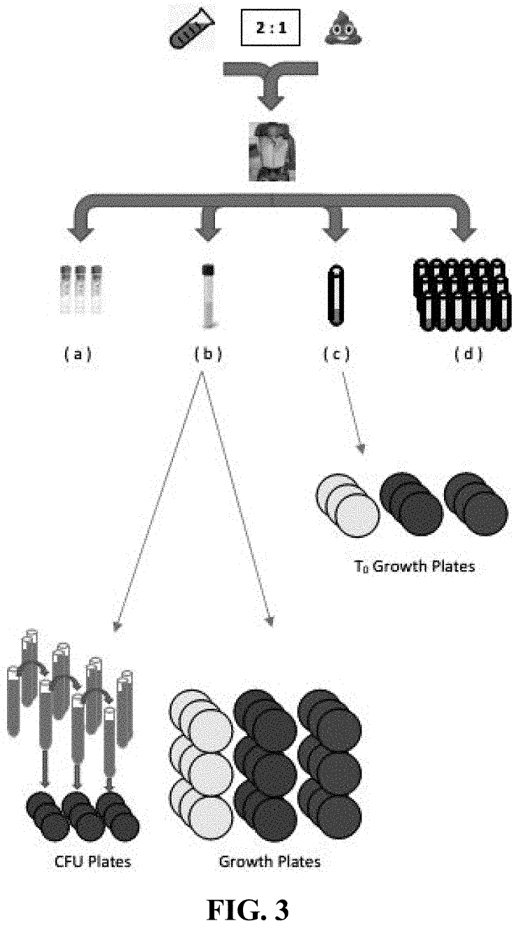

A stool sample from a human subject was collected and brought into an anaerobic chamber (containing a gas mixture of 5% CO₂/5% H₂/90% N₂) within 5 minutes of sample collection. The stool sample was then diluted with 2-parts pre-chilled (4° C.), pre-reduced, anaerobically sterilized (PRAS) dilution blank medium (AS-9183, Anaerobe Systems, Morgan Hill, Calif.) supplemented with 20% glycerol to 1-part stool sample and homogenized with a blender (FIG. 3).

### Stool Processing

As further illustrated in FIG. 3, (a) three replicate samples of homogenized stool were frozen for whole genome sequence (WGS); (b) homogenized stool was diluted 10⁻⁴ in YCFAC broth and plated (using 100 μL on a 10-cm plate using a hockey stick spreader) directly on three types of culture medium or serially diluted and then plated on YCFAC+B plates to determine CFU; (c) homogenized stool was added to a collection tube in an anaerobic atmosphere, incubated for 1 hour at 25° C., diluted 10⁻⁴ in TCFAC broth, and plated on three types of culture medium to ascertain the immediate effects of the storage formulation (a T=0 (To) control); or (d) set aside for further processing (storage and freezing) in formulation.

Culture Recovery from Fresh Stool Control (Step (b) in FIG. 3)

Homogenized fresh stool was diluted 10⁴-fold in YCFAC+B media. Aliquots were spread onto 3 replicate plates of solid media of each media type (BHI, CHOC, YCFAC+B) for high density growth plates (3 replicate sets of media were inoculated, 3 plates per media type). The 10⁻⁴ diluted stool was serially diluted and 10⁻⁵ to 10⁻⁷ dilutions were spread on YCFAC+B agar to determine a colony-forming unit (CFU) count. In this step, 3 replicate serial dilutions were performed. Each dilution was inoculated onto 3 plates (FIG. 4A). Plates were then incubated anaerobically at 37° C. for 120 hours.

Culture from T₀ Formulation Control (Step (c) in FIG. 3)

Homogenized fresh stool (1 g) was added to a collection tube containing 9 mL of formulation inside an anaerobic chamber and held at room temperature for 1 hour. This control step was conducted to evaluate whether the ingredients of the formulation had a negative effect on microorganisms in the stool samples. Subsequently, the mixture was diluted to a final dilution factor of 10⁻⁴ from whole stool in YCFAC broth, then spread on 3 replicate plates of each media type (BHI, CHOC, YCFAC+B) as illustrated in FIG. 3 to culture viable microorganisms and serially diluted and plated on YCFAC+B media for CFU determination (not shown).

### Sample Collection in the Formulation (Step (d) in FIG. 3)

The homogenized stool and formulation tubes were brought into air (FIG. 4A). Formulation tubes were inoculated with stool in air by removing the cap from the formulation tubes, adding stool to the formulation such that a final ratio of 1:10 stool to formulation was achieved, re-sealing the preserve tubes, and then shaking the tubes to mix and aerate. 18 replicate tubes were inoculated and stored.

### Sample Storage and Processing

The 18 formulation tubes were stored at room temperature for 72, 96, 120 hours, respectively, with 6 tubes per time point. Thus, at each storage timepoint, 6 formulation tubes were removed, and 3 formulation tubes were brought into an anaerobic chamber and processed for anaerobic culture recovery. The other 3 remaining formulation tubes were stored for an additional 7 days at −80° C., brought into an anaerobic chamber to thaw, and then processed for anaerobic culture recovery (FIG. 4A). WGS was performed on the 3 formulation tubes that were not frozen; the freeze-thawed samples were not sequenced (see, TABLE 5).

Culture Recovery from Formulation Samples

For each time point, (i) room temperature (“RT”) and (ii) room temperature+freeze-thaw stored (“FRZ” of freeze) formulation samples were serially diluted and spread onto solid YCFAC+B agar to count colony-forming units (CFU) and analyze organisms growing as isolated colonies. 3 sets of replicate dilutions were performed with 3 plates per dilution factor (growth plates, FIG. 4B). The room temperature and freeze-thaw stored formulation samples from each time point were also diluted with a dilution factor of 10⁻² in YCFAC broth (equivalent to a 10⁻³ dilution from fresh stool) and spread on 3 replicate plates of each media type (BHI, CHOC, YCFAC+B) for high density growth (growth plates, FIG. 4B). Thus, each sample was inoculated at high density on 3 media types with 3 replicate plates per media type and at low density on YCFAC+B). Low density plating favors the recovery of slow-growing species, as they are able to grow into isolated colonies with less competition for nutrients and less inhibition by waste products produced by fast-growing species. No significant variation in species type, diversity, and abundance was observed between the 3 replicates in this study.

### Harvesting Cells for Whole Genome Sequencing (WGS)

Microorganisms were harvested for WGS after 120 hours of growth in an anaerobic incubator. After serial dilution, plating, and growth, colonies on plates with 30-300 colonies were counted for CFU determination. Microorganisms from plates inoculated with 10-fold more stool (300-3000 colonies) were harvested for WGS as shown in FIG. 5A. Cells from 3 replicate plates per sample were harvested and combined into a single sample for WGS such that 3 replicate sets of harvested cells from each timepoint were obtained. Next, the colonies from high density growth plates were harvested and combined as shown in FIG. 5B. Similar to the CFU plate procedure, cells from 3 replicate plates of each media type, within a sample, were harvested and combined into a single sample for WGS such that 3 replicate sets of harvested cells from each timepoint were obtained.

### Sequencing

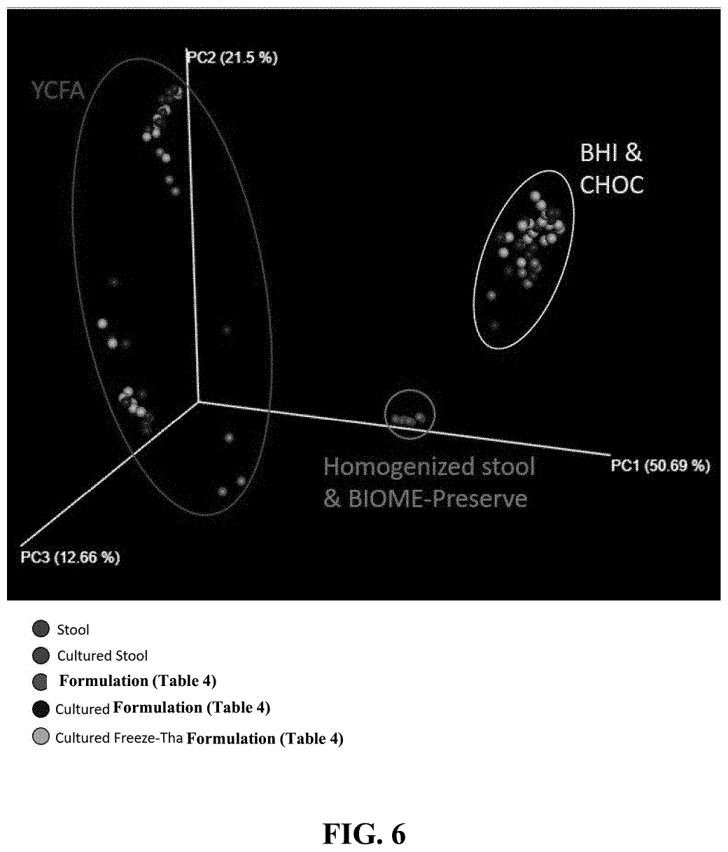

Whole genome shotgun sequencing was performed using an Illumina NovaSeq 2×150 flow cell with a targeted mean read depth of 2 million sequences per sample. Host (e.g., human) DNA sequences were removed from the data. Data were further filtered and de-noised by removing low-count taxa and low-coverage samples from dataset. The number of counts for each species/OTU was normalized to the OUT's genome length, and OTUs accounting for <one-millionth of all species-level markers across the entire data set were removed prior to further analysis. Sequence coverage excluded OUT's with <0.01% of their unique genome regions or <1% of their total genome. Moreover, samples with <10,000 sequences were removed prior to further analysis. A rarefied OTU table (10,000 reads/sample) was used for alpha diversity and beta diversity (Bray-Curtis PCoA) analyses (see, e.g., FIG. 6).

Moreover, for comparative analysis to determine culturable species, only OTUs with a taxonomic classification at the species level were included in the OTU table. If the relative abundance of a species was at least 0.05% in culture material collected from growth or CFU plates, that species was considered culturable. A species did not have to be detected on all growth and CFU plates to be considered culturable; a single instance of detection at or above 0.05% relative abundance was sufficient for a species to be considered recovered.

TABLE 5 below summarizes samples and replicates analyzed by whole genome shotgun sequencing. Fresh stool samples comprising 1 mL of fresh stool diluted in 2 mL formulation were homogenized and frozen at −80° C. Preserved stool samples comprising a 1 mL sample in 9 mL formulation were held at room temperature for the specified time period. The harvested bacterial cells described in TABLE 5 were from culture plates after 120 hours incubation. Cells from 3 replicate plates of the same media type were combined and mixed with 2 mL of PRAS Dilution Blank medium to create one WGS sample.

TABLE 5
Whole Genome Shotgun Sequencing Samples & Replicates

| Sample Time Point | Source Material | WGS Sample Replicates |
|---|---|---|
| Fresh Stool Control | Fresh Stool | 3 |
| | Cells Harvested from BHI | 3 |
| | Cells Harvested from CHOC | 3 |
| | Cells Harvested from YCFAC + B | 3 |
| | Cells Harvested from YCFAC + B (CFU) | 3 |
| Formulation (T₀ | Cells Harvested from BHI | 1 |
| Control) | Cells Harvested from CHOC | 1 |
| | Cells Harvested from YCFAC + B | 1 |
| Formulation (72 | Preserved Stool | 3 |
| Hour Room- | Cells Harvested from BHI | 3 |
| Temperature) | Cells Harvested from CHOC | 3 |
| | Cells Harvested from YCFAC + B | 3 |
| | Cells Harvested from YCFAC + B (CFU) | 3 |
| Formulation (96 | Preserved Stool | 3 |
| Hour Room- | Cells Harvested from BHI | 3 |
| Temperature) | Cells Harvested from CHOC | 3 |
| | Cells Harvested from YCFAC + B | 3 |
| | Cells Harvested from YCFAC + B (CFU) | 3 |
| Formulation (120 | Preserved Stool | 3 |
| Hour Room- | Cells Harvested from BHI | 3 |
| Temperature) | Cells Harvested from CHOC | 3 |
| | Cells Harvested from YCFAC + B | 3 |
| | Cells Harvested from YCFAC + B (CFU) | 3 |
| Formulation (72 | Cells Harvested from BHI | 3 |
| Hour Freeze-Thaw) | Cells Harvested from CHOC | 3 |
| | Cells Harvested from YCFAC + B | 3 |
| | Cells Harvested from YCFAC + B (CFU) | 3 |
| Formulation (96 | Cells Harvested from BHI | 3 |
| Hour Freeze-Thaw) | Cells Harvested from CHOC | 3 |
| | Cells Harvested from YCFAC + B | 3 |
| | Cells Harvested from YCFAC + B (CFU) | 3 |
| Formulation (120 | Cells Harvested from BHI | 3 |
| Hour Freeze-Thaw) | Cells Harvested from CHOC | 3 |
| | Cells Harvested from YCFAC + B | 3 |
| | Cells Harvested from YCFAC + B (CFU) | 3 |

### Example 7

### Evaluation of Microbial Preservation Formulations

This example describes the validation and performance evaluation of a collection formulation and storage device system.

### Determination of the Number of OTUs Under Various Storage Conditions

First, the number of operational taxonomic units (OTUs) in formulation preserved samples was measured under various storage conditions and compared to a fresh sample. FIG. 6 shows a Bray-Curtis Based Principal Coordinates (PC) Analysis and shows that both media type and dilution factor had an effect on culture recovery, wherein media type had an effect on what was recovered. These data include each media type tested, all incubation time points (72, 96, and 120 hours), and both room temperature and freeze-thawed samples. FIG. 6 further shows that sample recovery from various time points were grouped within each media type. Overall, samples within each time point group and media group were tightly grouped together, suggesting overall high data quality. More specifically, these data demonstrated that (i) fresh stool samples and stool in formulation preserved samples form a single cluster along PC1, indicating similar microbial communities between the two sample types; (ii) wider variation was noted between cultured bacteria samples on different media, suggesting different media formulations recovered different organisms; (iii) CHOC and BHI agar recovered similar organisms; (iv) YCFAB+C agar recovered a unique set of organisms compared to CHOC and BHI; (v) high dilution before plating on YCFAC+B agar yielded different microbial communities compared to bacterial mats; and (vi) variation in bacterial recovery due to culture media type was larger than variation between fresh stool and stool held in formulation preserved samples.

Next, the alpha diversity in stool samples was analyzed, and the observed OTUs of fresh stool sample and samples stored in collection formulation were compared. Alpha diversity refers to the average species diversity in a habitat or specific area. Alpha diversity is a local measure. 10,000 sequences were analyzed per sample for the studies in this section.

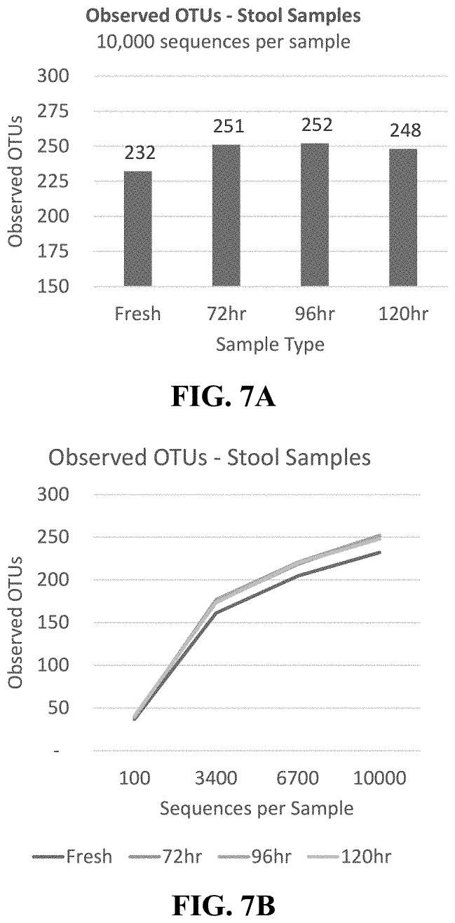

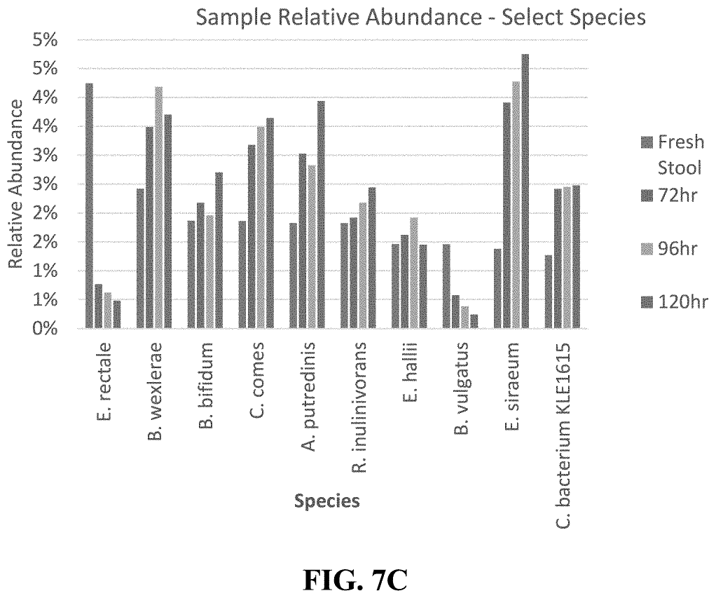

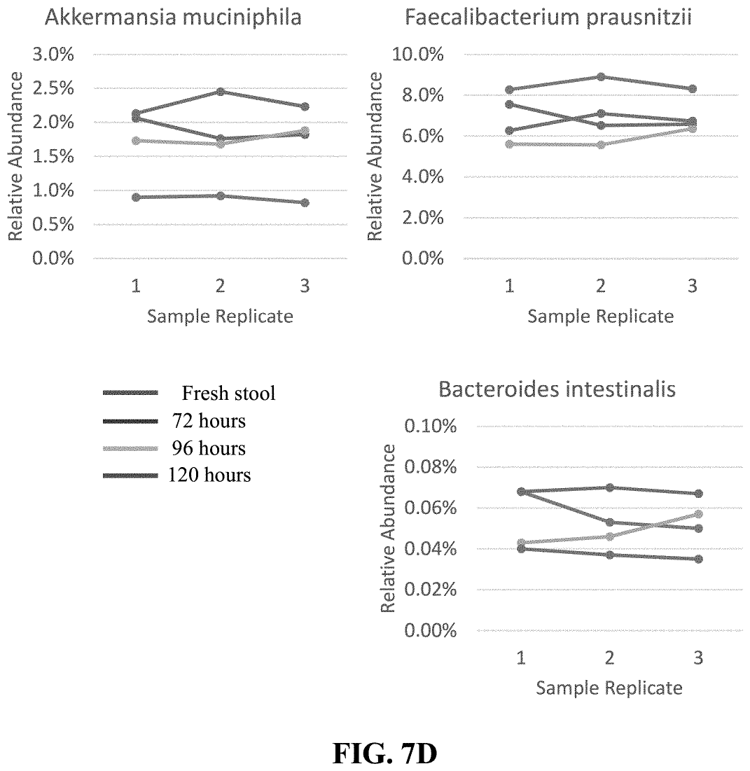

FIG. 7A shows that stool samples stored in collection formulation for 72, 96, and 120 hours, respectively, showed higher observed OTUs compared to the fresh stool sample. Without being bound by any theory, it was assumed that some microorganisms continued to multiply during storage in the formulation leading to an increased number of observed OTUs of formulation stored samples. Moreover, FIG. 7B shows that the number of observed OTUs increases with increased sequencing depth for fresh samples and samples stored in formulation for 72, 96, and 120 hours. FIG. 7C shows that the measured relative abundance of many species of microorganisms was similar in samples from formulation stored for 72, 96, and 120 hours, respectively, compared to those from fresh (not stored) sample. Moreover, no significant variation in species type, diversity, and abundance was observed between the 3 replicates in this study. FIG. 7D shows the relative abundance for the selected species Akkermansia muciniphila, Faecalibacterium prausnitzii (both high relative abundance), and Bacteroides intestinalis (low relative abundant species) in samples from fresh stool sample and from formulation samples stored for 72, 96, and 120 hours, respectively. These data demonstrate minimal cross-replicate variability for various species during storage for at least 5 days (120 hours).

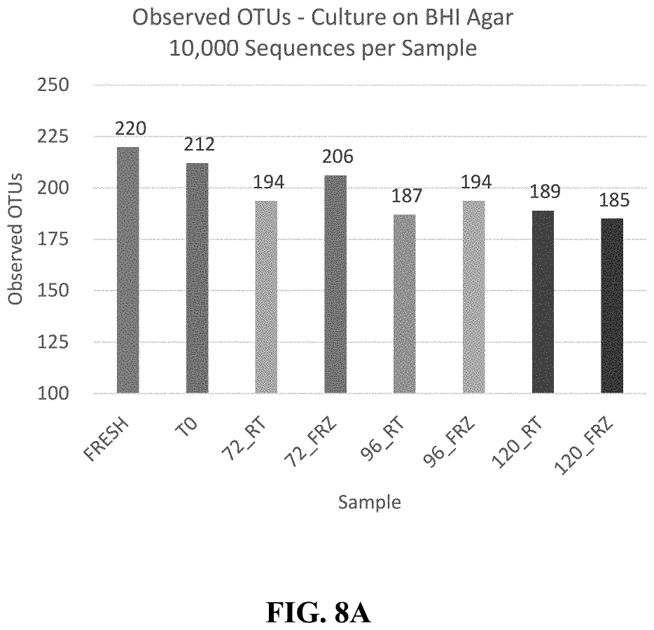

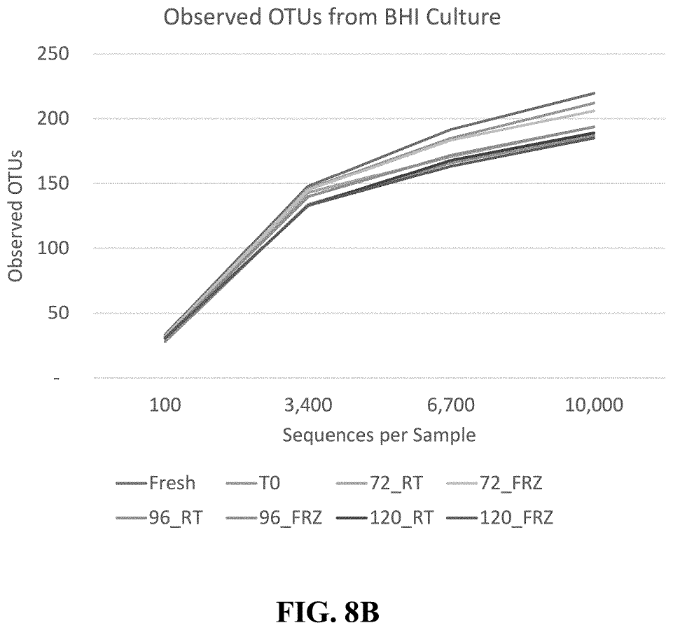

Subsequently, the alpha diversity of BHI Agar culture recovery samples was analyzed, and the observed OTUs of fresh stool sample and samples stored in collection formulation (formulation composition described in TABLE 4) were compared. FIG. 8A shows that stool samples were stored in formulation for 72, 96 and 120, respectively, either at room temperature only (samples abbreviated as “RT”) or storage at room temperature for the respective time periods followed by a period of 7 days during which the samples were held frozen (abbreviated as “FRZ”) followed by thawing (e.g., “freeze-thawing”), as described in EXAMPLE 6. The data showed comparable OTUs for fresh or To samples, indicating that (i) the formulation allows for storage for at least 120 hours followed by additional storage at freezing temperature, e.g., −80° C., without significantly reducing the number of viable microorganisms in a stool sample, and (ii) enables the culture and growth of microorganisms obtained from such stool sample. FIG. 8B shows that stool samples stored in formulation for 72, 96, and 120 hours, respectively, and that underwent either storage at room temperature or freeze-thawing as described in EXAMPLE 6 showed comparable OTUs per sequences per sample compared to fresh or To samples, further demonstrating the formulation and tube device was effective in keeping microorganisms, particularly anaerobic microorganisms, viable and growable for at least 120 hours.

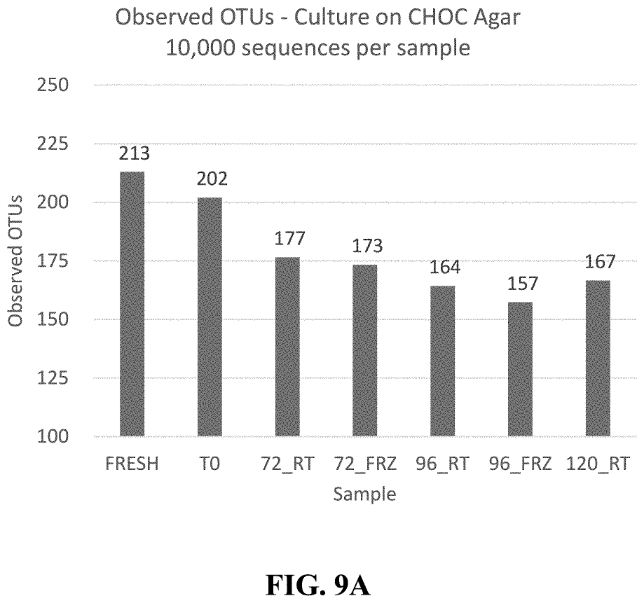

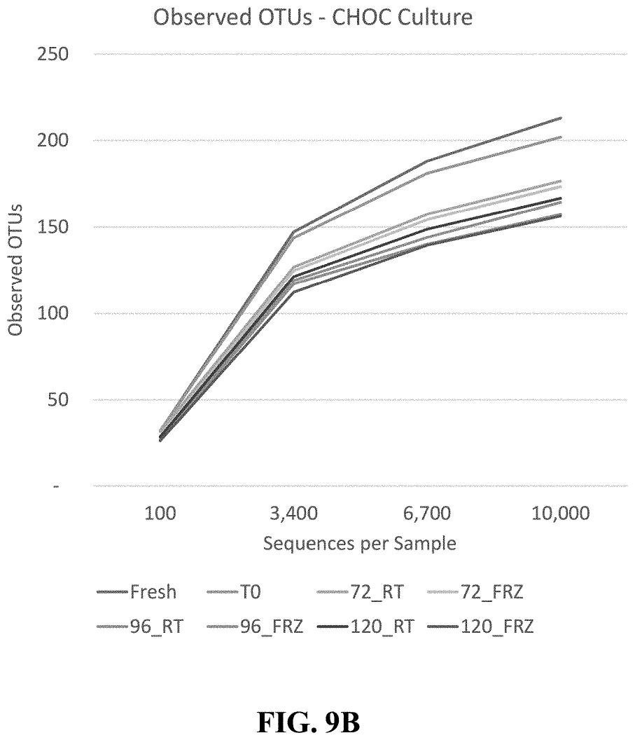

Similar results were obtained for the alpha diversity studies of CHOC Agar culture recovery samples. FIG. 9A shows that stool samples stored in formulation for 72, 96, and 120 hours, respectively, and that underwent storage at room temperature (abbreviated as “RT”) or further freeze-thawing (abbreviated as “FRZ”) as described in EXAMPLE 6 showed slightly reduced OTUs compared to fresh or To samples. However, even the 96-hour time point, freeze-thawed sample yielded 157 OTUs compared to 213 OTUs of the fresh sample, which was a reduction from the maximum of about 26%. FIG. 9B shows that stool samples stored in formulation for 72, 96, and 120 hours, respectively, and that underwent storage at room temperature or further freeze-thawing as described in EXAMPLE 6 showed slightly reduced OTUs per sequences per sample compared to fresh or To samples. However, even at the 120-hour time point, freeze-thawed sample yielded just over 150 OTUs per 10,000 sequences per sample, suggesting that the formulation and tube device allows for storage and culture of microorganisms of stool samples.

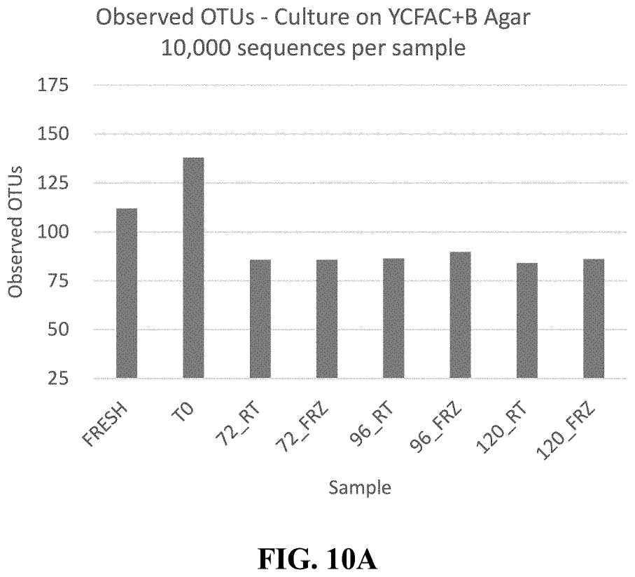

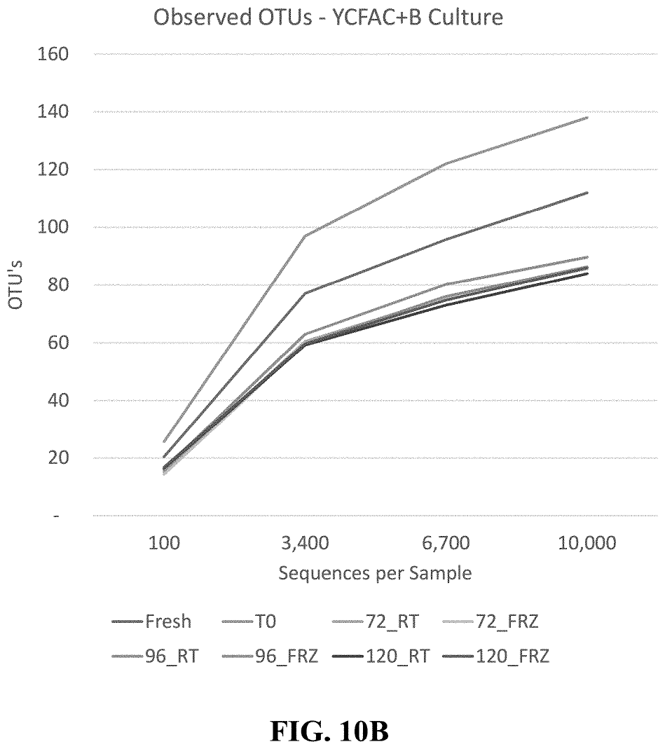

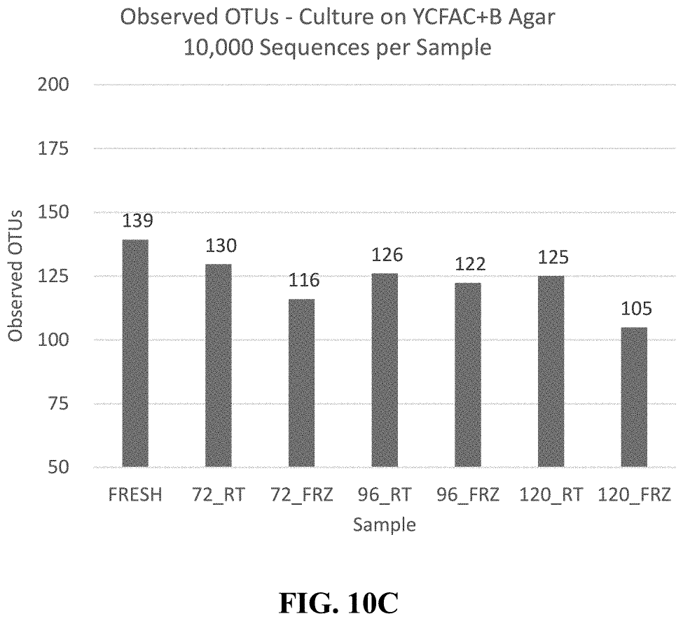

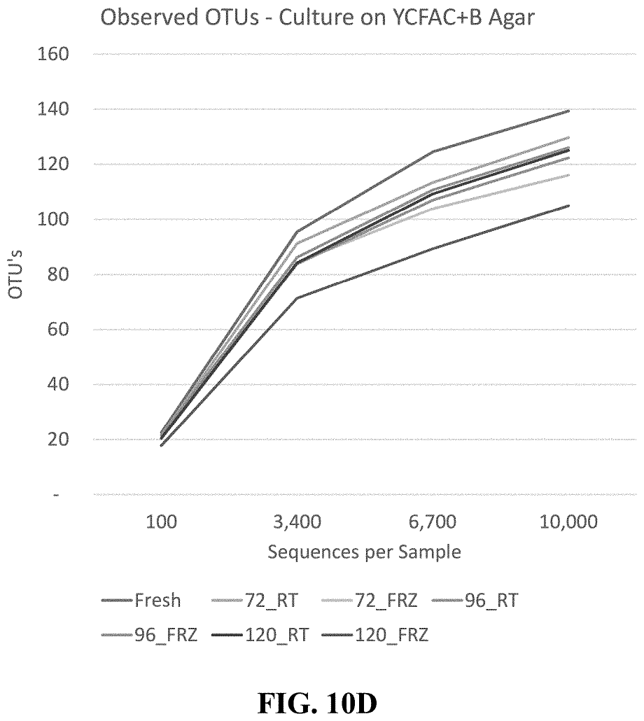

Results of alpha diversity studies performed on YCFAC+B Agar culture recovery samples are shown in FIGS. 10A-B. FIG. 10A shows that that stool samples stored in formulation for 72, 96, and 120 hours, respectively, and that underwent either storage at room temperature only (abbreviated as “RT”) and those that underwent additional freeze-thawing (abbreviated as “FRZ”) as described in EXAMPLE 6 showed slightly reduced OTUs compared to fresh or To samples. However, even the 120-hour time point, freeze-thawed sample yielded about 85 OTUs compared to about 110 OTUs of the fresh sample, which was a reduction from the maximum of about 23%. FIG. 10B shows that stool samples stored in formulation for 72, 96, and 120 hours, respectively, showed slightly reduced OTUs per sequences per sample compared to fresh or To samples. However, even at the 120-hour time point, freeze-thawed sample yielded just over 80 OTUs per 10,000 sequences per sample, suggesting that the formulation and tube device allows for storage and culture of microorganisms of stool samples. The alpha diversity results in high dilution YCFAC+B Agar medium are shown in FIGS. 10C-D and demonstrate that the formulation (composition described in TABLE 4) effectively preserved the cells, providing OTUs at the three time points measured that were not significantly lower than those observed for the fresh sample.

### Species Relative Abundance in Fresh Stool and Stool Stored in Formulation

First, the change in relative abundance for the 20 top most abundant species in the stool sample was determined. Overall, about 100 species accounted for about 99% of the organisms which were identified at bacterial species level in this study. FIG. 11A shows that the relative abundance of most of the top 20 most abundant species was not significantly reduced over the 120-hour storage time using the formulation. More specifically, no significant change in the relative abundance of 18 of the top 20 most abundant species was observed. FIG. 11B and FIG. 11C show similar results for the change in relative abundance for the middle 20 species (i.e., species 40-60) of the 100 most abundant species, and the bottom 20 species (i.e., species 80-100) of the 100 most abundant species, respectively. Overall, the number of species present above 0.1% relative abundance was 48 in the fresh stool sample, 57 after storage for 72 hours, 56 after storage for 96 hours, and 58 after storage for 120 hours. The number of species present above 0.01% relative abundance was 130 in the fresh stool sample, 128 after storage for 72 hours, 126 after storage for 96 and 126 after storage for 120 hours, as summarized in the following table:

| | Relative | Relative |
|---|---|---|
| Storage | abundance | abundance |
| time | >0.1% | >0.01% |
| Fresh | 48 | 130 |
| 72 hours | 57 | 128 |
| 96 hours | 56 | 126 |
| 120 hours | 58 | 126 |

FIG. 11D illustrates these observations by showing the number of abundant (>0.10%), low abundant (0.01%-0.1%) and rare (<0.01%) species recovered from culture combined across all media types for fresh sample and sample that was stored in formulation at room temperature for 72 hours, 96 hours, and 120 hours, respectively. These data demonstrate that, compared to fresh sample, the formulation preserved the majority of low-abundance and rare species across time points and up to at least 5 days. FIG. 11E further illustrates a bar graph showing the number of recovered species with a relative abundance of >0.05% cultured from fresh sample and formulation stored (for 72, 96, and 120 hours) samples for all four culturing conditions (BHI, CHOC, YCFAC+B, and CFU YCFAC+B). These data demonstrate only minimal or no loss of culturable diversity for all 4 culturing conditions and up to 5 days. These data were compiled from combined sequencing results of triplicate bacterial mats scraped from BHI, CHOC, YCFAC+B, and CFU YCFAC+B plates.

Overall, these data demonstrate that the collection formulation (and container) can be used to preserve bacterial consortia in stool samples for at least 120 hours and allows them to be cultured following storage in the collection formulation described herein.

### Example 8

### Culture Recovery, Processing, and Analysis of Formulation Stored Microorganisms

This example describes the cell culture recovery, processing and analysis of microorganisms of a stool sample that were stored in a collection formulation (composition described in TABLE 4).

A stool sample was stored in a collection formulation and container for 72 hours at room temperature.

### Culture Protocol

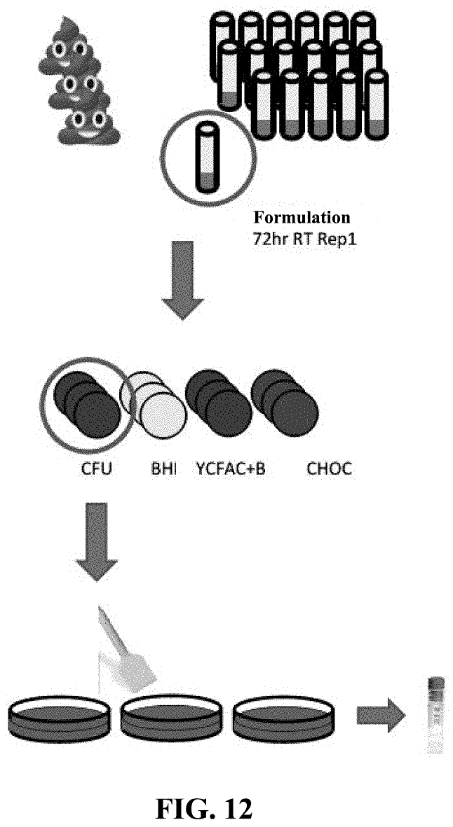

The culture protocol of this study included 3 culture media types covering 4 culture conditions: Brain Heart Infusion Agar (BHI), Chocolate Agar (CHOC), Yeast Casitone Fatty Acid with Carbohydrate+Blood Agar (YCFAC+B), and CFU count plates on YCFAC+B (FIG. 12). Each sample was set up on all 4 culture conditions, with 3 replicate plates per sample. Following storage at room temperature for 72 hours, 100 μL of the diluted stool sample in collection formulation were added to Agar plates of the respective each medium and culture conditions using 10⁻² dilution for general plates and 10⁻⁵ for CFU plates, wherein the fresh stool samples were diluted 10⁻⁴ for general plates and 10⁻⁷ for CFU plates. Agar plates were then incubated anaerobically at 37° C. for 120 hours. It was observed that general growth plates (i.e., the 10⁻² and 10⁻⁴ dilutions) grew as solid mat of bacteria, whereas the CFU plates (i.e., the 10⁻⁴ and 10⁻⁷ dilutions) grew with approximately 800-1,000 isolated colonies.

### Harvesting

Following incubation for 120 hours, the cells were harvested from plates with a cell scraper and cells from the 3 replicate plates within a single sample (for each media condition) were combined with 2 mL of PRAS dilution blank medium. About 1 mL of harvested cell suspension was frozen at −80° C. for DNA extraction, processing, and WGS.

### Analysis

Whole-genome shotgun sequencing was performed using an Illumina NovaSeq 2×150 flow cell with a targeted mean read depth of 2 million sequences per sample. To filter and de-noise data, low-count taxa and low-coverage samples were removed from the analysis. For comparative analysis to determine culturable species, only OTUs with a taxonomic classification at the species level were included in the OTU table. If the relative abundance of a species was at least 0.05% in culture material collected from growth or CFU plates, that species was considered culturable. A species did not have to be detected on all growth and CFU plates to be considered culturable; a single instance of detection at or above 0.05% relative abundance was sufficient for a species to be considered recovered.

Culture from Fresh Stool (Control)

TABLE 6 below shows the growth of bacteria obtained from fresh stool control samples across all media types and conditions:

TABLE 6
Bacterial growth across all media types and conditions

| | BHI | CHOC | YCFAC + B | CFU |
|---|---|---|---|---|
| # of Species Cultured | 69 | 69 | 36 | 48 |
| # of Species “Exclusively” Cultured | 7 | 11 | 13 | 16 |
| Total # Species Cultured | 117 | | | |
| # of Species Recovered Only from | 47 | | | |
| a Single Media Condition | | | | |

Overall, from the 117 species that were cultured across all conditions, 29 of the species were found in low relative abundance in stool (present with <0.01%) and 14 of the species were found to be rare in stool (present with <0.001%). Moreover, 47 species grew only under one culture condition and recovery on the same media within replicate sets was consistent for high density growth plates, but more variable for CFU plates.

Growth analysis on BHI Media showed that 69 species cultured from fresh stool spread on BHI agar and that 7 species grew only on BHI, and not on the other media conditions (“Exclusively Cultured”). Growth analysis on CHOC Media showed that 69 species cultured from fresh stool spread on CHOC agar and that 11 species grew only on CHOC, and not on the other media conditions.

Growth analysis on YCFAC+B Media showed that 36 species cultured from fresh stool spread on YCFAC+B agar and that 13 species grew only on YCFAC+B, and not on the other media conditions.

Growth analysis of isolated Colonies on YCFAC+B Media (CFU plates) showed that from 48 species cultured (>0.05% relative abundance in culture), that 16 species grew only as isolated colonies, and not on the other media conditions, and that most rare and low relative abundance species in stool were not recovered on the CFU plates. However, it is noted that a greater variability of stochastic distribution was observed for low abundance species, and that use of a different culture medium may allow recovery of different low abundance species.

Comparison of Culture from Fresh Stool and to Formulation Control

Next, the cultures from fresh stool were compared to the T₀ time point of formulation preserved stool sample (these samples were stored at room temperature for 1 hour in the respective media). Such samples were processed for storage but were maintained in storage media for only 1 hour. TABLE 7 shows the species that were cultured from fresh stool and the T₀ formulation samples on various media:

TABLE 7
Species Cultured from Fresh Stool &
T₀ Samples on Various Media

| | T₁ₕᵣ | Fresh Stool Sample | | |
|---|---|---|---|---|
| | Sample | R1 | R2 | R3 |
| Species Recovered on BHI | 60 | 58 | 57 | 65 |
| Species Recovered on CHOC | 62 | 62 | 62 | 65 |
| Species Recovered on YCFAC + B | 42 | 36 | 32 | 34 |

* The parameters R1, R2, and R3 refer to the different replicates within one culture condition

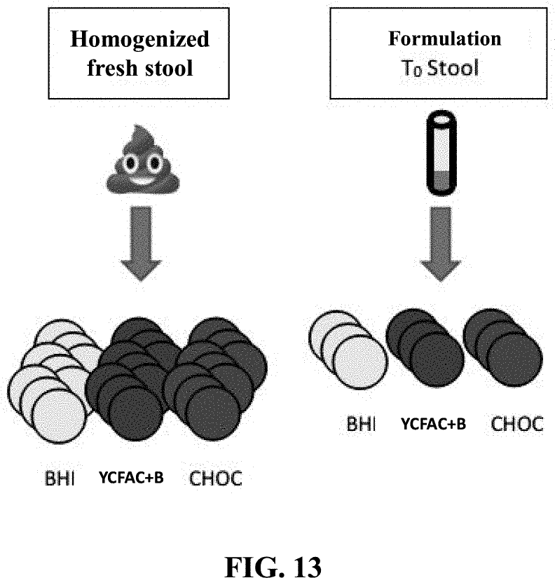

The T₀ culture conditions included homogenizing fresh stool that was then added to formulation inside an anaerobic chamber and held anaerobically for 1 hour (FIG. 13). The 1-hour held sample was then plated on 3 media types (BHI, CHOC, YCFAC+B) with 3 replicate plates per media type. The Formulation did not appear to have inhibitory or negative effect on culture recovery from fresh stool. Additionally, the organisms which had a measurable increase in relative abundance in T₀ formulation preserved samples compared to fresh stool samples were later successfully cultured in 72/96/120 hour held formulation samples.

Growth analysis on BHI Media showed that 69 species cultured from fresh stool spread on BHI agar compared to 60 species that were cultured from stool held for 1 hour in formulation.

Growth analysis on CHOC Media showed that 69 species cultured from fresh stool spread on CHOC agar compared to 62 species that were cultured from stool held for 1 hour in formulation.

Growth analysis on YCFAC+B Media showed that 36 species cultured from fresh stool spread on YCFAC+B agar compared to 42 species that were cultured from stool held for 1 hour in formulation.

Culture from 72-Hour Formulation Preserved Stool Samples

TABLE 8 below shows the species that were cultured from the 72-hour formulation samples compared to those that were cultured from fresh stool:

TABLE 8
Species Cultured from the 72-hour
Formulation vs. Fresh stool

| | Form- | Fresh |
|---|---|---|
| # of Species Cultured | ulation | Stool |
| Total # of Species | 127 | 117 |
| # of “Low Relative Abundance” Species | 36 | 29 |
| # of “Rare” Species | 15 | 14 |
| # of Species Not Recovered vs. Fresh | 8 | — |
| Recovery on various media types | | |
| # of Species from BHI Agar | 77 | 69 |
| # of Species from CHOC Agar | 66 | 69 |
| # of Species from YCFAC + B Agar | 36 | 36 |
| # of Species from CFU Plates | 50 | 48 |

As described above, from the 117 species that were cultured across all conditions from fresh sample, 29 of the species were found in low relative abundance in stool (<0.01%) and 14 of the species were found to be rare in stool (<0.001%). In comparison, 127 species were cultured across all conditions from the 72-hour formulation samples, wherein 36 of the species were found in low relative abundance in stool (<0.01%) and 15 of the species were found to be rare in stool (<0.001%). According to the data shown in TABLE 8, on average 93% of species cultured from fresh stool were preserved in formulation, demonstrating the viability of the microbial preservation media described herein. Moreover, and as shown in TABLE 8, storage of stool sample in formulation allowed culturing of organisms that were not culturable from fresh stool sample. Such species were not included in the 93% average of recovered species from formulation preserved samples.

When the cultures of the four different conditions were analyzed for the 72-hour formulation samples, it was found that (i) 77 species cultured in the HBI media samples (compared to 69 species for the fresh sample control); (ii) 66 species cultured in the CHOC media samples (compared to 69 species for the fresh sample control); (iii) 36 species cultured in the YCFAC+B media samples (compared to 36 species for the fresh sample control); and that (iv) 50 species were cultured as isolated colonies in YCFAC+B medium (compared to 48 species for the fresh sample control.

Culture from 96-Hour Formulation Preserved Stool Samples

TABLE 9 below shows the species that were cultured from the 96-hour formulation samples compared to those that were cultured from fresh stool:

TABLE 9
Species Cultured from the 96-hour
Formulation vs. Fresh stool

| | Form- | Fresh |
|---|---|---|
| # of Species Cultured | ulation | Stool |
| Total # of Species | 112 | 117 |
| # of “Low Relative Abundance” Species | 32 | 29 |
| # of “Rare” Species | 15 | 14 |
| # of Species Not Recovered vs. Fresh | 16 | — |
| Recovery on various media types | | |
| # of Species from BHI Agar | 73 | 69 |
| # of Species from CHOC Agar | 59 | 69 |
| # of Species from YCFAC + B Agar | 32 | 36 |
| # of Species from CFU Plates | 41 | 48 |

As described above, from the 117 species that were cultured across all conditions from fresh sample, 29 of the species were found in low relative abundance in stool (<0.01%) and 14 of the species were found to be rare in stool (<0.001%). In comparison, using the formulation 112 species were preserved and cultured across culturing conditions from the 96-hour formulation samples, wherein 32 of such species were in low relative abundance (<0.01%) and 15 of the species were rare (<0.001%) in the stool sample. According to the data shown in TABLE 9, on average 86% of bacterial species from the stool sample were preserved and cultured after 96 hours of storage in formulation.

Growth analysis on BHI media showed that after 96-hour storage in formulation 73 bacterial species were successfully cultured compared to 69 species from the fresh stool sample control.

Growth analysis on CHOC media showed that after 96-hour storage in formulation 59 bacterial species were successfully cultured compared to 69 species from the fresh stool sample control.

Growth analysis on YCFAC+B media showed that after 96-hour storage in formulation 32 bacterial species were successfully cultured compared to 36 species from the fresh stool sample control.

Growth analysis of isolate colonies on YCFAC+B media showed that after 96-hour storage in formulation 41 bacterial species were successfully cultured compared to 48 species from the fresh stool sample control.

Culture from 120-Hour Formulation Preserved Stool Samples

TABLE 10 below shows the species that were cultured after storage for 120 hours in the formulation of TABLE 4 compared to those that were cultured from fresh stool:

TABLE 10
Species Cultured after storage
for 120 hours vs. Fresh stool

| | Form- | Fresh |
|---|---|---|
| # of Species Cultured | ulation | Stool |
| Total # of Species | 108 | 117 |
| # of “Low Relative Abundance” Species | 35 | 29 |
| # of “Rare” Species | 15 | 14 |
| # of Species Not Recovered vs. Fresh | 22 | — |
| Recovery on various media types | | |
| # of Species from BHI Agar | 71 | 69 |
| # of Species from CHOC Agar | 61 | 69 |
| # of Species from YCFAC + B Agar | 31 | 36 |
| # of Species from CFU Plates | 41 | 48 |

As described above, from the 117 species that were cultured across all conditions from fresh sample, 29 of the species were found in low relative abundance in stool (<0.01%) and 14 of the species were found to be rare in stool (<0.001%). In comparison, using the formulation 108 species were preserved and cultured across culturing conditions from the 120-hour samples, wherein 35 of such species were in low relative abundance (<0.01%) and 15 of the species were rare (<0.001%) in the stool sample. According to the data shown in TABLE 10, on average 81% of bacterial species from the stool sample were preserved and cultured after 120 hours of storage in formulation.

Growth analysis on BHI media showed that after 96-hour storage in formulation 71 bacterial species were successfully cultured compared to 69 species from the fresh stool sample control.

Growth analysis on CHOC media showed that after 96-hour storage in formulation 61 bacterial species were successfully cultured compared to 69 species from the fresh stool sample control.

Growth analysis on YCFAC+B media showed that after 96-hour storage in formulation 31 bacterial species were successfully cultured compared to 36 species from the fresh stool sample control.

Growth analysis of isolate colonies on YCFAC+B media showed that after 96-hour storage in formulation 41 bacterial species were successfully cultured compared to 48 species from the fresh stool sample control.

The data demonstrate that the formulation can be used to preserve microorganisms for at least 120 hours following sample collection.

Culture Recovery after Freeze-Thaw

Next, the culture recovery of species was determined after freeze-thawing. The freeze-thaw conditions and percent recovered species for these conditions are shown in TABLE 11 below.

TABLE 11
Freeze-Thaw Conditions and Percent Recovered Species

| # Species Cultured | 72 hr | 96 hr | 120 hr |
|---|---|---|---|
| Room Temperature/ | 127 (11) | 112 (4) | 108 (12) |
| (# exclusive species, not | | | |
| cultured when Freeze- | | | |
| Thawed) | | | |
| Freeze-Thaw/ | 119 (3) | 118 (10) | 104 (8) |
| (# exclusive species, | | | |
| not cultured at RT) | | | |
| % Recovery of species | 91% | 96% | 89% |
| from freeze-thaw culture | | | |

The recovery rate across all media types and freeze-thaw conditions was about 89%-96%. A portion of species not recovered after freeze-thaw were in low or rare relative abundance in stool. Furthermore, some species which were not recovered in the room-temp stored samples were recovered in the freeze-thaw samples. Overall, the data show that most of the organisms recovered from freeze-thawed samples were in low or rare relative abundance in stool.

For samples that were stored for 72 hours at room temperature it was found that 127 species were cultured across all conditions from room-temp samples, which includes 11 species that were recovered from room-temp but not from freeze-thawed samples. For samples that were freeze-thawed for 72 hours it was found that 119 species were cultured across all conditions from room-temp samples, which includes 3 species that were recovered from freeze-thawed samples but not samples stored at room temperature. Overall, the data show that 91% of species cultured from room-temperature samples were recovered from the freeze-thaw samples.

For samples that were stored for 96 hours at room temperature it was found that 112 species were cultured across all conditions from room-temp samples, which includes 4 species that were recovered from room-temp but not from freeze-thawed samples. For samples that were freeze-thawed for 96 hours it was found that 118 species were cultured across all conditions from room-temp samples, which includes 10 species that were recovered from freeze-thawed samples but not samples stored at room temperature. Overall, the data show that 96% of species cultured from room-temperature samples were recovered from the freeze-thaw samples.

For samples that were stored for 120 hours at room temperature it was found that 108 species were cultured across all conditions from room-temp samples, which includes 12 species that were recovered from room-temp but not from freeze-thawed samples. For samples that were freeze-thawed for 120 hours it was found that 104 species were cultured across all conditions from room-temp samples, which includes 8 species that were recovered from freeze-thawed samples but not samples stored at room temperature. Overall, the data show that 89% of species cultured from room-temperature samples were recovered from the freeze-thaw samples.

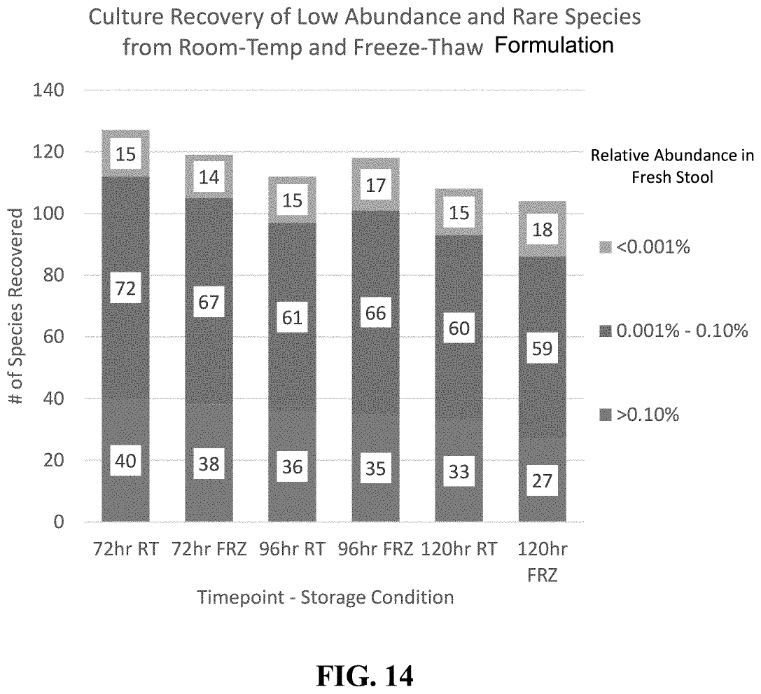

FIG. 14 further demonstrates that freeze-thawing samples that were stored at room temperature in formulation does not significantly affect the recoverable species. This figure shows the total of species recovered with >0.05% relative abundance from formulation preserved samples stored at room temperature only compared to formulation preserved samples stored at room temperature (for 72, 96, and 120 hours, respectively) then frozen for 7 days and then thawed.

Overall, these data demonstrate that the anaerobic collection formulations and devices herein are capable of preserving the viability and abundance of anaerobic microorganisms in a biological sample. The kits, methods, and systems provided herein can be used to not only store but also culture anaerobic microorganisms following storage and transport. This can be used to analyze the microbiome samples of a large number of subjects from various locations as well as to use isolates of such samples to provide therapeutic microbial compositions for the treatment and/or prevention of microbiome-related disorders.

### Example 9

### Species Relative Abundance and Culturable Diversity in Fresh Stool Compared to Formulation-Preserved Stool Samples

This example shows that the formulations described herein can be used to store biological samples (e.g., stool samples) containing bacteria, such as anaerobe bacteria, and preserve such bacteria for up to at least 120 hours with only minimal loss in the number and diversity of viable bacterial species compared to viable bacterial species in a fresh sample.

### Experimental Study Procedures

### Stool Sample Collection and Processing

For this study, stool samples from three healthy donors were obtained. Three adult (18+ years of age) human donors volunteered to provide a single whole stool sample for this study. The donors were asked to provide a sample using a commode specimen collection bucket (02-544-208, Fisher Scientific, Hampton, N.H.). The samples were immediately processed upon arrival. Donors included one adult female in her 30s, one adult male in his 70s, and one adult male in his 40s with a verbally self-reported prior recurrent C. difficile infection. All donors self-reported verbally no active gastrointestinal infections or recent antibiotic usage at the time of sample collection.

The stool samples were brought into an anaerobic chamber containing a gas mixture of 5% CO₂/5% H₂/90% N₂ within 30 minutes, 4 hours, and 1 hour of sample collection for donors 1, 2, and 3, respectively. Inside the anaerobic chamber, whole stool was diluted with 2-parts chilled (4° C.) and pre-reduced anaerobically sterilized PRAS Dilution Blank medium supplemented with 20% glycerol to 1-part stool and then homogenized with a blender (a Ninja 400-watt blender (QB900B Shark Ninja, Needham, Mass.)). Subsequently, three 0.5 mL aliquots of homogenized stool were collected into 2 mL cryovials (#431386, Corning Life Sciences, Corning, N.Y.) and immediately frozen at −80° C. One cryovial was sent for WGS sequencing and analysis, the other was maintained in frozen storage, and the remaining stool sample was diluted to achieve a 10⁻⁵ dilution in YCFAC broth to create the culture inoculum: 3.0 mL homogenized stool was transferred to a 7 mL YCFAC broth tube for a 10⁻¹ dilution. 1.0 mL of the 10⁻¹ dilution was transferred to a 9 mL PRAS Dilution Blank tube for a 10⁻² dilution. 778 μL of the 10⁻² dilution was transferred to a new 7 mL YCFAC broth tube for a 10⁻³ dilution. 71 μL of the 10⁻³ dilution was transferred to a new 7 mL YCFAC broth tube for a 10⁻⁵ dilution. The 10⁻⁵ dilution was then used in a serial dilutions for CFU determination and for culturing on solid media as described below. A first portion of homogenized stool was added into a sample container (e.g., a sample tube) for use as a T₀ control sample. A second portion of homogenized stool was set aside for inoculation into the formulation shown in TABLE 4.

Culture from T₀ Control Samples

For cell culture from the T₀ control sample, 1 mL of homogenized fresh stool was added to 9 mL formulation inside an anaerobic chamber and held at room temperature for 1 hour. After that, the sample mixture was diluted to a final dilution factor of 10⁻⁵ using YCFAC broth, and then spread on 3 replicate plates of each media type, BHI, CHOC, YCFAC+B media. To that end, a T₀ formulation control tube was inoculated after homogenizing fresh stool to assess for any inhibitory effect of the components contained in the formulation on the viability of culturable organisms from stool. Homogenized stool (1.0 mL) was added directly to a single formulation tube (9 mL) then held at room temperature for 1 hour. Inoculation and holding were performed inside of an anaerobic chamber eliminate any confounding effects due to oxygen. After one hour, a sequential dilution of the T₀ formulation tube into YCFAC broth was performed: 3.0 mL To formulation contents were transferred into a 7 mL YCFAC broth tube to create a 10⁻² dilution, then 778 μL of the 10⁻² dilution was transferred to a new 7 mL YCFAC broth tube to create a 10⁻³ dilution, and finally 71 μL of the 10⁻³ dilution was transferred to a new 7 mL YCFAC broth tube to create a 10⁻⁵ dilution. The 10⁻⁵ dilution was then used for high density and low-density culturing on solid media as described above for fresh stool. A single replicate set of media were inoculated for the T₀ formulation control sample.

Culture Recovery from Fresh Stool Control Samples

For culture recovery from stool control samples, homogenized fresh stool was serially diluted, targeting 10⁻⁶-10⁻⁸ dilutions and performed twice to create two serial dilution replicate sets, and then spread on BRU and YCFAC+B agar for low density growth and colony-forming unit (CFU) determination using 2 replicate serial dilutions and 3 growth plates per dilution factor. For high-density growth, diluted fresh stool with a dilution factor of 10⁻⁵ was spread onto 3 replicate growth plates of each media type, BRU, CHOC, YCFAC+B, in two replicate sets (i.e., 6 plates per medium). The growth plates were then incubated for 120 hours for growth. Photographs were taken of each plate, and colonies were counted for the plates in the countable range of 30-300 colonies. After 120 hours of incubation, bacterial colonies were carefully removed from the 10⁻⁶ plates using the beveled edge of cell lifter (08-100-240, Fisher Scientific, Hampton, N.H.) and transferred to a 2 mL cryovial. These plates had approximately 1,000 isolated colonies. Harvested cells from the three replicate plates from a single dilution replicate were combined into a single cryovial. 2 mL of dilution blank were added to the cryovials, the cellular material was mixed via mechanical mixing, pipet mixing, and repeated inversion of the tube, and 1 mL of material was transferred to a new 2 mL cryovial. All cryovials were frozen immediately at −80° C.; one of each duplicate tube was sent for WGS sequencing and analysis.

### Sample Collection, Storage, and Processing Using Formulation

Homogenized stool sample and a sample collection container tube for receiving the stool sample were brought into air, followed by placing the stool sample into the container by (i) removing the cap from the container tube, (ii) placing 1 mL of homogenized stool sample and 9 mL of formulation into the container, and (iii) re-sealing the tube, followed by mixing and aerating the sample mixture. Four replicate sample tubes were generated for each of the three donors.

The samples tubes were stored at room temperature for 72 and 120 hours, respectively. At each timepoint, 2 sample tubes were removed from the storage area and brought into an anaerobic chamber for anaerobic culture recovery. A portion of each stored sample was also prepared for WGS.

### General Experimental Processes

Sample tubes were inoculated outside of the anaerobic chamber to mimic traditional sample collection in air. At the end of each specified holding period, a 0.5 mL aliquot was collected from each formulation tube and stored in a 2 mL cryovial at −80° C. The contents of a single replicate formulation tube per timepoint was sent for WGS sequencing analysis. Only a single formulation sample was sequenced because data from prior experimentation showed little technical variation between replicate samples. Formulation tubes were inverted to mix contents, 217 μL were transferred to each of two replicate 7 mL YCFAC broth tubes to create a 10⁻³ YCFAC formulation dilution from stool. The 10⁻³ dilution was then used in serial dilutions for CFU determination and for culturing on solid media as described below. The formulation tubes were processed for low density culture and CFU determination as follows: To determine the CFU of fresh stool and for inoculating low-density growth plates, we performed serial dilution, targeting 10⁻⁴-10⁻⁶ dilutions from stool. The 10⁻⁴-10⁻⁶ dilutions were inoculated (100 μL) onto BRU and YCFAC+B solid media plates. Three replicate plates of each media type were inoculated per dilution. The plates were then incubated in the anaerobic chamber at 37° C. for 120 hours. CFU determination and harvesting of the 10⁻⁴ dilution plates were done in the same manner as described above fresh stool culture.

Formulation tubes were processed for high density culture recovery as follows: 100 μL of 10⁻³ YCFAC formulation dilution created previously were added to triplicate plates of each of the three media types inoculated with fresh stool (YCFAC+B, CHOC, BRU). Plates were incubated anaerobically at 37° C. for 120 hours and then contents were harvested and stored in cryovials as described above for fresh stool culture plates. A 10⁻³ formulation dilution was chosen as compared to a 10⁻⁵ dilution from fresh stool to account for expected overall 2-factor reduction in CFU during storage in formulation.

Culture Recovery from Stool Samples Stored in Formulation

Stool samples stored in formulation were serially diluted and spread on BRU and YCFAC+B agar for high dilution microorganism (e.g., bacteria) recovery and CFU determination. For each stored stool sample, 2 replicate dilution sets were performed using 3 plates per dilution factor. The stored samples were diluted 10⁻³ in YCFAC broth (i.e., 10⁻³ dilution from fresh stool) and spread onto solid media (BRU, CHOC and YCFAC+B) for high density organism recovery.

Thus, to determine which microbes could be recovered from fresh stool using varying media types, 100 μL of the 10⁻⁵ YCFAC stool dilution were streaked onto triplicate plates of the following solid media: YCFAC+B, PRAS Chocolate Agar (CHOC, AS-244, Anaerobe Systems, Morgan Hill, Calif.), and BRU. Two replicate sets of media were inoculated. The plates were incubated anaerobically for 120 hours at 37° C. Following the procedure for the CFU plates described above, bacterial mat material was removed from the plates, transferred to 2 mL cryovials, and split into two cryovials for a final suspended volume of 1 mL. All cryovials were frozen immediately at −80° C.; one of each duplicate cryovial was sent for WGS sequencing and analysis. Thus, one set of media was inoculated for each formulation-preserved stool sample and each set included 3 media types and 3 replicate plates per media type.

### Cell Harvesting, Sequencing, and Analysis

Bacterial colonies were harvested from CFU plates as follows: (i) high dilution culture plates with approximately 1,000 isolated colonies were set aside for harvesting cells; (ii) cells from the 3 replicate plates within a sample were harvested and combined into a single sample for WGS; and (iii) 2 replicate sets of harvested cells were collected from each timepoint.

Bacterial colonies were harvested from high density growth plates as follows: (i) cells from the 3 replicate plates of each media type, within a sample, were harvested and combined into a single sample for WGS; and (ii) 2 replicate sets of harvested cells for each media type were collected for each sample.

DNA extraction was performed using a QIAGEN PowerSoil Pro DNA isolation kit. BoosterShot whole genome shotgun sequencing included (i) an Illumina NovaSeq 2x150 flow cell; (ii) a targeted mean read depth of 2 million sequences/sample; (iii) BRU high density and low-density plates for Donor 1 underwent an additional 20 million sequences/sample deep sequencing (DeepSeq) analysis; and (iv) host (e.g., human) sequences were removed prior to processing and analysis. (See, e.g., Hillman et al., Evaluating the Information Content of Shallow Shotgun Metagenomics, mSystems, 2018 Nov. 13; 3(6):e00069-18).

The bacterial cell sequences were processed as follows: (i) the number of counts for each operational taxonomic unit (OTU, e.g., bacterial species) was normalized to the OTU's genome length, and OTUs accounting for <one-millionth of all species-level markers across the entire data set were removed prior to further analysis; (ii) OTUs with <0.01% of their unique genome regions or <1% of their total genome covered were removed prior to further analysis; and (iii) samples with <10,000 sequences were removed prior to further analysis. The same filtering process was applied to DeepSeq sequencing data. DeepSeq data were used as an extra layer of data to determine recovered organisms as described below and to validate cutoff values for deeming a species to be present or not; all diversity and taxonomic analyses were performed on BoosterShot data.

To determine culturable species, only OTUs with a taxonomic classification at the species level were included in the OTU table. If the relative abundance of a species was at least 0.01% in culture material, that species was considered culturable. A species did not have to be detected on all culture plates to be considered culturable; if the average relative abundance across a single set of media plate replicates was at least 0.01% relative abundance, that species was considered culturable (recovered). The data showed that species making up at least 0.01% relative abundance from cultured cells were preserved and recovered because of CoreBiome analysis and the low proportion that the original inoculum represented within the harvested cells.

On high density growth plates, stool was diluted to 10⁻⁵; 100 μL of the dilution was spread on the plate. Assuming stool contains approximately 10¹⁰ CFU/mL, the inoculum on the plate would be approximately 10⁴ cells. Bacteria grew in a dense solid mat on agar plates, which was harvested for analysis. Assuming that the harvested bacteria were made up of approximately 10⁹ cells, the inoculum would account for 0.001%. Nearly all species' relative abundance in stool was <10%, so an individual species' percentage in the inoculum would be expected to be less than 0.0001% of the harvested cells, thus making it unlikely that it would be detected if it were not truly recovered in culture.

The term “impacted species” is used herein for species which appeared to have negatively impacted viability after storing in formulation. Because of the large variability inherent in culture from a dilute and highly diverse microbial community, it may be challenging to determine whether species did not grow because it was not preserved alive, or if it were alive and would be recovered if more replicate cultures plates were analyzed.

A species was considered impacted if the relative abundance in culture from formulation samples had a 1-factor (10⁻¹) reduction as compared to culture from fresh stool. To determine the number of impacted species, it was first determined which species were successfully cultured from fresh stool, and then compared each species' relative abundance in fresh stool culture to its relative abundance in formulation culture and determined the percentage drop in relative abundance. Species with at least >90% drop were considered impacted. We did not include any bacterial species in these determinations that fit all of the following criteria: relative abundance in stool <0.01%, relative abundance in the formulation sample <0.01%, and relative abundance of fresh stool culture <0.05%. Due to the combination of low relative abundance in the sample and in culture recovery we felt we could not determine whether the inherent variability from dilution, sampling, and growth accounted for the drop or if those species were truly impacted.

In instances where a species was present at 0.01% relative abundance in BoosterShot data, DeepSeq data (donor 1 only) indicated whole genome coverage for at least one strain of each species was between 2.3% to 97%, depending on the species (the range was 1.1%-65% for unique genomic region coverage). Given these results, together with the estimated inoculum, the data suggested that species detected at a relative abundance of at least 0.01% in BoosterShot data grew on culture media. It may have been possible that some species with a relative abundance lower than 0.01% also grew successfully, but it may be challenging to classify such species as recovered or not with the collected data.

### Study Results

In this study, stool samples from three healthy donors were obtained, processed and analyzed as described in above in this example.

First, the diversity of bacterial communities was observed to differ across donors (see, e.g., TABLE 12 below). Using three different culture media and two dilution factors, 173 species from donor 1, 168 species from donor 2, and 168 species from donor 3, were cultured from fresh stool. Importantly, several of these species are of interest to the microbiome community, including several Bifidobacterium and Bacteroides species, Akkermansia muciniphila, and Faecalibacterium prausnitzii. The diversity of species present across the donors provided a foundation to test the ability of the formulation (TABLE 4) to preserve a variety of bacterial species across different individual donors.

TABLE 12
Relative Abundance of Selected Bacteria
in Stool Samples of all 3 Donors

| | Donor 1 | Donor 2 | Donor 3 |
|---|---|---|---|
| Species | Stool | Stool | Stool |
| Faecalibacterium prausnitzii | 12.2% | 7.5% | 24.9% |
| Prevotella copri | 0.0% | 9.7% | 6.1% |
| Blautia obeum | 2.1% | 4.7% | 2.8% |
| Eubacterium rectale | 4.7% | 3.4% | 0.6% |
| Coprococcus eutactus | 0.4% | 4.4% | 3.1% |
| Fusicatenibacter saccharivorans | 4.5% | 0.4% | 2.2% |
| Roseburia faecis | 2.8% | 0.0% | 3.8% |
| Eubacterium hallii | 2.9% | 2.1% | 1.0% |
| Bifidobacterium adolescentis | 5.2% | 0.0% | 0.7% |
| Collinsella aerofaciens | 1.1% | 1.0% | 3.7% |
| Alistipes putredinis | 0.6% | 3.6% | 1.4% |
| Akkermansia muciniphila | 0.0% | 5.3% | 0.0% |
| Bifidobacterium bifidum | 0.0% | 4.3% | 0.6% |
| Coprococcus comes | 1.4% | 2.1% | 0.8% |
| Dorea longicatena | 1.2% | 2.1% | 0.7% |
| Ruminiclostridium Eubacterium | 2.5% | 0.0% | 0.1% |
| siraeum | | | |
| Roseburia inulinivorans | 0.2% | 1.1% | 1.2% |
| Bacteroides vulgatus | 1.2% | 0.5% | 0.8% |
| Bifidobacterium | 0.0% | 1.8% | 0.5% |
| pseudocatenulatum | | | |
| Parabacteroides merdae | 0.7% | 0.1% | 1.5% |
| Blautia Ruminococcus gnavus | 0.1% | 1.9% | 0.1% |
| Enterorhabdus caecimuris | 0.0% | 1.9% | 0.0% |
| Anaerostipes hadrus | 0.5% | 1.2% | 0.2% |
| Alistipes finegoldii | 0.9% | 0.9% | 0.0% |
| Eubacterium ramulus | 0.2% | 0.8% | 0.8% |

Before assessing the capacity of the formulation used in this example to maintain anaerobic organisms alive for culture, we first determined whether the additives in the formulation which components are set forth in TABLE 4 harmed anaerobic organisms from human stool. We sequenced cultured cells from homogenized stool collected from donor 1, as well as stool held in formulation for 1 hour. It is important to note that this tube, unlike the other formulation tubes, was inoculated and held under anaerobic conditions to eliminate any harmful effects of temporary exposure to oxygen and ensure the only variables tested were the formulation additives.

As shown in TABLE 13 below, there was only minimal difference in overall diversity of the total number of cultured species from the formulation tubes held for 1 hour compared to fresh homogenized stool. A total of 163 species were recovered on culture media from the T₀ formulation tube, compared to 173 species recovered from fresh, homogenized stool. The distribution of species with various relative abundance in stool was also maintained from culture recovery of the T₀ formulation tube as compared to culture from fresh stool. These results indicate that the components of the formulation do not harm anaerobic organisms in fresh stool.

TABLE 13

| | Stool Culture | T₀ Culture |
|---|---|---|
| # Species | 173 | 163 |
| Recovery Rate | | 94% |
| Relative Abundance in Stool | Stool Culture | T₀ Culture |
| <0.01% | 46 | 37 |
| 0.01-0.1% | 78 | 76 |
| >0.1% | 49 | 50 |
| Total | 173 | 163 |
| | Stool Culture | T₀ Culture |
| Not Considered Impacted | 173 | 165 |
| Impacted: 1-factor drop | — | 6 |
| Impacted: 2-factor drop | — | 2 |

| | |
|---|---|
| Proportion Not Impacted | 95% |

Next, it was determined whether inoculation in air and longer holding times had any effect on the anaerobes that could be cultured from formulation compared to anaerobes that could be cultured from fresh stool. Formulation tubes (i.e., sample containers comprising stool sample and formulation) were inoculated in air with homogenized stool and held at room temperature for two time periods (72 hours and 120 hours), chosen for relevance to typical lengths of time it can take for samples to arrive in the laboratory from the field, or to transit from one location to the next for processing. While microbiome samples may be either frozen for long-term storage or processed within 72 hours of collection, formulation tubes were tested for up to 5 days to mimic longer transit times due to delays in transporting samples from the field to the laboratory, choosing less expensive shipping methods, delayed delivery, or other shipping issues.

Serial dilutions followed by plating on BRU and YCFAC+B agar plates to yield isolated colonies revealed an initial two-factor drop in bacterial CFUs from formulation held for 72 hours in the formulation of TABLE 4 compared to fresh stool, without any further loss upon storage for 120 hours. Despite this loss in absolute bacterial numbers, the majority of culturable species from stool experienced minimal changes in both the number of species recovered and the drop in relative abundance after storage in and culture from storage formulation across all three donors.

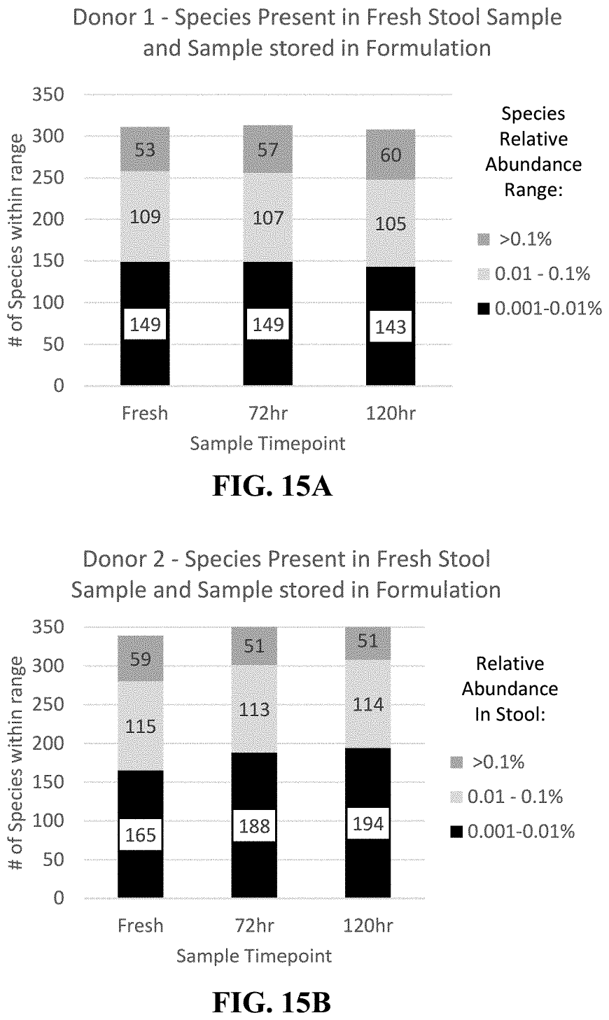

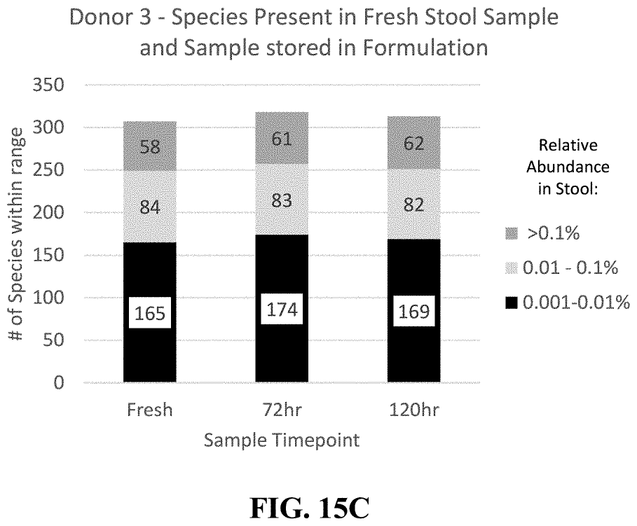

FIG. 15A-FIG. 15C and TABLE 14 below show that there was only minimal loss in the numbers of species detected after storage in formulation for all three donors compared to the fresh stool sample. The detected species were categorized according to their relative abundance in fresh stool, and the results show a similar degree of preservation for abundant (>0.1% relative abundance), low abundance (0.01-0.1% relative abundance), and rare (0.001-0.01% relative abundance) species. Overall species diversity, represented by total number of species recovered, was minimally impacted as compared to culture from fresh stool. TABLE 14 below summarizes these data for all 3 donors:

TABLE 14

| Relative | | | | | | | | | |
|---|---|---|---|---|---|---|---|---|---|
| Abundance | Number of Species Detected in Fresh and Stored Stool | | | | | | | | |
| Distribution | Donor 1 | | | Donor 2 | | | Donor 3 | | |
| in Stool | Fresh | 72 hr | 120 hr | Fresh | 72 hr | 120 hr | Fresh | 72 hr | 120 hr |
| >0.1% | 53 | 57 | 60 | 59 | 51 | 51 | 58 | 61 | 62 |
| 0.01-0.1% | 109 | 107 | 105 | 115 | 113 | 114 | 84 | 83 | 82 |
| 0.001-0.01% | 149 | 149 | 143 | 165 | 188 | 194 | 165 | 174 | 169 |
| Total | 311 | 313 | 308 | 339 | 352 | 359 | 307 | 318 | 313 |

Next, the diversity of culturable species in fresh stool samples was compared to stool samples stored in the formulation of TABLE 4. TABLE 15 shows that the formulation was capable of maintaining the majority of culturable species diversity compared to a fresh stool sample. Averaged across all donors, 94% of diversity was maintained after 72 hours and 89% maintained after 120 hours. TABLE 15 shows the total number of species that were recovered with a relative abundance of >0.01% from (i) fresh stool and (ii) stool sample stored in formulation for 72 and 120 hours, across all media types. In some instances (e.g. Donor 1, 120 hours) the number of culturable low abundance or rare species actually increased after storage, which was expected due to the natural variation in successful culture of low-abundance organisms. The data show that species diversity of culturable bacteria was not significantly affected during storage in the formulation for all 3 donors. TABLE 15 below summarizes these data for all 3 donors:

TABLE 15

| | # Species Recovered (Recovery Rate) | | |
|---|---|---|---|
| | Fresh | 72 Hour | 120 Hour |
| Donor 1 | 173 | 163 (94%) | 183 (106%) |
| Donor 2 | 168 | 145 (86%) | 133 (79%) |
| Donor 3 | 168 | 169 (101%) | 139 (83%) |
| Average | 170 | 159 (94%) | 152 (83%) |

### Relative Abundance Maintained in Formulation

The results of this study further show that the culturable viability of relatively few species was negatively impacted by storage for up to 120 hours. A microorganism species (e.g., a bacterial species or OTU) was considered impacted if its relative abundance in a culture prepared from a stored sample was reduced by 90% (1-factor impact) or 99% (2 factor impact) compared to its relative abundance in a culture prepared from fresh stool. TABLE 16 shows, for example, that 88%, 82%, and 87% of species recovered donors 1, 2, and 3, respectively were not impacted after storage in formulation for 72 hours. TABLE 16 below summarizes these data for all 3 donors:

TABLE 16

| | Species Cultured from Fresh Stool | | | | | | | | |
|---|---|---|---|---|---|---|---|---|---|
| | Donor 1 | | | Donor 2 | | | Donor 3 | | |
| | Fresh | 72 hr | 120 hr | Fresh | 72 hr | 120 hr | Fresh | 72 hr | 120 hr |
| Minimal Impact | 173 | 153 | 147 | 168 | 138 | 142 | 168 | 149 | 135 |
| 1-Factor Impact | | 13 | 21 | | 29 | 21 | | 16 | 20 |
| 2-Factor Impact | | 7 | 5 | | 1 | 5 | | 3 | 13 |

Across all donors an average of 86% of species were minimally impacted after storage in formulation for 72 hours, and 83% were minimally impacted after 120 hours, as shown in TABLE 17 below:

TABLE 17
Average Impacted Species Across All Donors

| | Fresh | 72 hr | 120 hr |
|---|---|---|---|
| Minimal | 170 | 147 (86%) | 141 (83%) |
| Impact | | | |
| 1-Factor | — | 19 | 21 |
| Impact | | | |
| 2-Factor | — | 4 | 8 |
| Impact | | | |

The results of this study further demonstrate that culturable diversity was maintained during storage in the formulation of TABLE 4. The formulation maintained viability of the majority of low-abundance and rare species across time points in stored stool samples compared to fresh stool samples. For example, TABLE 18 shows the culture recovery of rare and low abundance species across all time points and for all 3 donors. The bar graph also highlights the proportion of species recovered categorized as (i) present (>0.1% relative abundance in stool), (ii) low-abundance (between 0.01% and 0.1% relative abundance in stool), and (iii) rare (<0.01% relative abundance in stool). TABLE 18 below further summarizes these data for all 3 donors

TABLE 18

| Relative | Number of Species Recovered from Samples Within Various Relative Abundances from Stool | | | | | | | | |
|---|---|---|---|---|---|---|---|---|---|
| Abundance | Donor 1 | | | Donor 2 | | | Donor 3 | | |
| in Stool | Fresh | 72 hr | 120 hr | Fresh | 72 hr | 120 hr | Fresh | 72 hr | 120 hr |
| <0.01% | 46 | 56 | 71 | 41 | 48 | 54 | 50 | 70 | 58 |
| | (27%) | (34%) | (39%) | (24%) | (33%) | (41%) | (30%) | (41%) | (42%) |
| 0.01%-0.1% | 78 | 61 | 64 | 72 | 52 | 38 | 64 | 49 | 39 |
| | (45%) | (37%) | (35%) | (43%) | (36%) | (29%) | (38%) | (29%) | (28%) |
| >0.1% | 49 | 46 | 48 | 55 | 45 | 41 | 54 | 50 | 42 |
| | (28%) | (28%) | (26%) | (33%) | (31%) | (31%) | (32%) | (30%) | (30%) |
| Total | 173 | 163 | 183 | 168 | 145 | 133 | 168 | 169 | 139 |

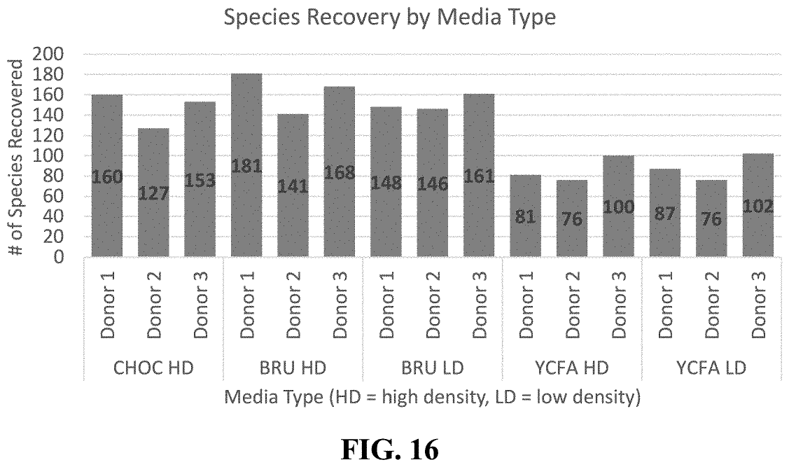

Further, and as shown in FIG. 16, a medium-dependent recovery of bacterial species was observed, with CHOC and BRU agar yielding similar microbial diversity and community composition to one another. Bacterial species recovery using YCFAC+B was lower compared to other media in terms of the number of species it was able to recover, but it was able to recover specific organisms that CHOC and BRU agar could not. Of note, using these media, including YCFAC+B, allowed the recovery of organisms at a very low relative abundance (<0.01%) in fresh, homogenized stool after holding/storage in formulation. This supports the need to use multiple media types to recover the majority of bacterial diversity.

Together, these data demonstrate that the formulations of the present disclosure, e.g., those shown in TABLES 1-4, can be used to store and preserve microorganisms, e.g., bacteria, present in a biological sample, e.g., a stool sample, for at least 5 days, and possible longer. The data further demonstrate that at least 75% of microorganisms of a biological sample can be preserved using a formulation described herein. Storage of a sample containing microorganisms in such formulations can also allow for subsequent cell culture of the microorganisms for further uses including sequencing analysis, production of live biotherapeutic products, and others. The data also show that storage in a formulation shown in TABLE 4 had minimal impact on viability. 86% of viable bacterial species were culturable after storage for 72 hours and 84% of viable bacterial species were culturable after storage 120 hours. The formulations herein can be used to store, preserve, and subsequently culture several species of particular interest, including Bifidobacterium spp., Bacteroides spp., Akkermansia muciniphila, and Faecalibacterium prausnitzii. Moreover, the data show that the kits and methods described herein are practical for collection, transport, and culture of anaerobic microbes from human samples, including stool samples. The culture conditions described herein can be used in further research and in the development of microbiome-based therapeutics.

These data further demonstrate that using the formulations described herein can be an effective method for preserving anaerobic bacteria for up to 5 days while preserving viability. From stool samples of 3 donors and using three media types and two dilution factors, an average of 170 species were cultured from fresh stool and detected via WGS sequencing. After 72 hours in formulation, it was possible to recover at least 94% of the number of species recovered in fresh stool, and about 89% after 120 hours storage in formulation. The formulation used in this example maintained microbial diversity and community composition from samples stored 72 and 120 hours.

In this study, formulation tubes were inoculated, capped, and stored in normal air. Further preservation may be afforded if stool is added to formulation inside of an anaerobic chamber. Because such an approach may not always be feasible, especially during field studies, the results shown here demonstrate what can be achieved in the most likely real-world scenario.

The impacted species metric is a useful tool to de-noise the recovery data and highlight species which were well preserved or negatively impacted. Cultures consisted of a vast and highly diverse population with many factors such as sampling variation, spreading, and inter-community growth affecting a species successful culture. Because of these, the recovery of a particular species may vary between samples even though there is little overall community variation. WGS analysis and laboratory resources make the utilization of many replicates cost prohibitive, thus analyzing which species had impacted recovery aids in measuring culture recovery performance. Despite that, several species of interest to microbiome researchers, including Faecalibacterium prausnitzii, Bifidobacterium bifidum, and Akkermansia muciniphila were successfully recovered from at least one donor. Species that showed lower recovery in these experiments may be cultured in different media, thereby potentially allowing to increase their recovery after storage in a formulation. Thus, it is noted that our results are based on the media chosen; additional species may be recoverable after storage if other media and targeted protocols are used for culture recovery. Of note, some organisms that may be targeted by streaking for isolation could be diluted out of the inoculum during serial dilution; therefore, plating from multiple dilutions may be recommended depending on the targeted species.

Moreover, it was demonstrated that not only did the formulation maintain 89% of culturable bacterial diversity compared to fresh stool after 120 hours (5 days) and 94% of culturable bacterial diversity compared to fresh stool after 72 hours (3 days), but also that low abundance (<0.01% relative abundance) species in fresh stool were successfully recovered after inoculation into formulation. The majority of species did not have impacted recovery. This is an especially important consideration for therapeutic applications such as fecal microbiota transplant (FMT), which likely use the introduced community to experience minimal changes in community structure and viability between sample collection and therapeutic delivery.

Altogether, our results show that the formulations disclosed in this application provide an effective tool for collecting and preserving human stool samples for culture recovery of anaerobic organisms in the laboratory. The ability of the formulations to preserve nearly 89% of culturable diversity after five days at room temperature demonstrates the utility of the formulation, kits, methods, and systems described herein for any microbiome study where it is impractical to immediately freeze or process samples, and will likely facilitate studies that until now have been infeasible.

### Example 10

### Effects of Long-Term Shelf Storage of Formulation on Organism Recovery

This example shows that the formulations provided in this disclosure can be stored for several month at room temperature in anaerobic packaging without significantly impacting their capability to preserve organisms (e.g., anaerobic bacteria) from a biological sample.

Two sample containers (tubes) containing the formulation consisting of the components shown in TABLE 4 were inoculated with stool sample, wherein one formulation was freshly produced (abbreviated below as “fresh” formulation) and the second formulation had been produced 5 months prior to this study and since stored at room temperature in its original anaerobic packaging (abbreviated below as “shelf-life” formulation). Both container types, i.e., one containing 5-month old formulation and the other one freshly produced formulation, were divided into 2 groups for storing the stool sample for 72 hours and 120 hours, respectively.

Culture recovery from 5-months shelf-stored formulation revealed that overall species diversity was merely minimally impacted as compared to culture from freshly made formulation. TABLE 19 shows that the total number of species recovered from fresh 72-hour formulation tubes were 163 as compared to 169 species recovered from the 5-months shelf-stored 72-hour formulation tube. For 120 hour held samples, 183 and 176 species were recovered from the freshly produced and the 5-months shelf-stored formulations, respectively. The older, 5-months shelf-stored formulation was also able to recover species with a wide range of relative abundance in stool, thereby maintaining the number of species recovered in multiple relative abundance groupings as compared to culture from freshly produced formulation.

TABLE 19

| | 72 Hour | | 120 Hour | |
|---|---|---|---|---|
| | Fresh | Shelf Life | Fresh | Shelf Life |
| #Species | 163 | 169 | 183 | 176 |
| Recovery | | | | |
| Recovery % | | 104% | | 96% |
| Relative | | | | |
| Abundance | | | | |
| In Stool | Fresh | Shelf Life | Fresh | Shelf Life |
| <0.01% | 56 | 60 | 71 | 61 |
| 0.01-0.1% | 61 | 61 | 64 | 68 |
| >0.1% | 46 | 48 | 48 | 47 |
| Total | 163 | 169 | 183 | 176 |

The data further show that culture recovery was only minimally impacted for a majority of bacterial species when comparing the 5-months shelf-stored and freshly produced formulations. For example, as shown in TABLE 20 below, 88% of culturable species in stool were minimally impacted after storage in fresh formulation and 84% were minimally impacted after storage in 5-months shelf-stored formulation after 72 hours of storage in the respective formulations. Moreover, those numbers were 85% for both fresh and 5-months shelf-stored formulations for the 120-hour time point.

TABLE 20

| | 72 Hour | | 120 Hour | |
|---|---|---|---|---|
| | | 5-months shelf- | | 5-months shelf- |
| | Fresh | stored | Fresh | stored |
| Minimally Impacted | 153 | 142 | 147 | 150 |
| Impacted: 1-factor | 13 | 20 | 21 | 19 |
| drop | | | | |
| Impacted: 2-factor | 7 | 7 | 5 | 7 |
| drop | | | | |
| Proportion not | 88% | 84% | 85% | 85% |
| Impacted | | | | |

These data demonstrate that the formulations described herein can be stored in anaerobic packaging for several months (e.g., at least 5 months and possibly longer) without affecting a formulation's ability to preserve microorganisms (e.g., anaerobic bacteria) of a biological sample for at least 120 hours.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1-99. (canceled)

100. A kit for storing a biological sample comprising at least one microorganism from a subject, comprising:
a formulation comprising an antioxidant and an oxygen scavenger; and
a container configured to store said biological sample and said formulation, which container is configured to be sealed;
wherein said formulation is not exposed to a headspace gas comprising more than 1% oxygen.

101. The kit of claim 100, wherein said antioxidant comprises ascorbic acid, glutathione, dithiothreitol, glutathione peroxidase, a peroxidase, superoxide dismutase, catalase, a derivative thereof, or any combination thereof.

102. The kit of claim 100, wherein said oxygen scavenger comprises thioglycolate, cysteine, glutathione, glutathione peroxidase, peroxidase, catalase, superoxide dismutase, or any combination thereof.

103. The kit of claim 100, wherein said formulation further comprises a cryoprotectant.

104. The kit of claim 103, wherein said cryoprotectant comprises glycerol.

105. The kit of claim 100, wherein said formulation further comprises an electrolyte.

106. The kit of claim 105, wherein said electrolyte comprises sodium chloride, potassium chloride, sodium phosphate, potassium phosphate, magnesium sulfate, or any combination thereof.

107. The kit of claim 100, wherein said formulation further comprises a metabolite scavenger.

108. The kit of claim 107, wherein said metabolite scavenger comprises activated carbon, glutathione, ascorbic acid, or a combination thereof.

109. The kit of claim 100, wherein said formulation comprises sodium thioglycolate, sodium phosphate dibasic, sodium chloride, potassium chloride, potassium phosphate monobasic, magnesium sulfate heptahydrate, L-cysteine, glycerol, glutathione, glutathione peroxidase, peroxidase, superoxide dismutase, catalase, and DL-dithiothreitol.

110. The kit of claim 100, wherein said biological sample comprises at least 10 different species of anaerobic microorganisms.

111. A method for preserving a biological sample comprising at least one microorganism, said method comprising:
(a) providing a container comprising a formulation comprising an antioxidant and an oxygen scavenger, wherein said container does not comprise a gas comprising more than 1% oxygen;
(b) placing said biological sample into said container; and
(c) sealing said container,
thereby preserving viability of said at least one microorganism for at least 2 days.

112. The method of claim 111, wherein said antioxidant comprises ascorbic acid, glutathione, dithiothreitol, glutathione peroxidase, a peroxidase, superoxide dismutase, catalase, a derivative thereof, or any combination thereof.

113. The method of claim 111, wherein said oxygen scavenger comprises thioglycolate, cysteine, glutathione, glutathione peroxidase, peroxidase, catalase, superoxide dismutase, or any combination thereof.

114. The method of claim 111, wherein said formulation further comprises a cryoprotectant.

115. The method of claim 111, wherein said formulation further comprises an electrolyte.

116. The method of claim 111, wherein said formulation further comprises a metabolite scavenger.

117. The method of claim 111, wherein said formulation comprises sodium thioglycolate, sodium phosphate dibasic, sodium chloride, potassium chloride, potassium phosphate monobasic, magnesium sulfate heptahydrate, L-cysteine, glycerol, glutathione, glutathione peroxidase, peroxidase, superoxide dismutase, catalase, and DL-dithiothreitol.

118. The method of claim 111, further comprising:
(d) depositing said container with a processing unit that processes said biological sample.

119. The method of claim 118, wherein said depositing comprises providing said container to a parcel delivery service that delivers said container to said processing unit.

120. The method of claim 111, further comprising, prior to (a), receiving a kit comprising one or more of said formulation, said container, and a collection device.

121. The method of claim 111, wherein the viability of at least 10 different species of microorganisms is preserved for at least 4 days.

122. The method of claim 111, wherein said method is capable of preserving the viability of Faecalibacterium prausnitzii, Prevotella copri, Blautia obeum, Eubacterium rectale, Coprococcus eutactus, Fusicatenibacter saccharivorans, Roseburia faecis, Eubacterium hallii, Bifidobacterium adolescentis, Collinsella aerofaciens, Alistipes putredinis, Akkermansia muciniphila, Bifidobacterium bifidum, Coprococcus comes, Dorea longicatena, Ruminiclostridium Eubacterium siraeum, Roseburia inulinivorans, Bacteroides vulgatus, Bifidobacterium pseudocatenulatum, Parabacteroides merdae, Blautia Ruminococcus gnavus, Enterorhabdus caecimuris, Anaerostipes hadrus, Ahstipes finegoldii, and Eubacterium ramulus

123. A method of culturing at least one anaerobic microorganism from a biological sample, comprising:
(a) receiving a container comprising (i) said biological sample and (ii) a formulation comprising an antioxidant, an oxygen scavenger, and a cryoprotectant;
(b) inoculating said biological sample or a portion of said biological sample on a culture medium; and
(c) incubating said culture medium in an anaerobic chamber for a period of at least six hours such that said at least one anaerobic microorganism of said biological sample grows on said culture medium,
wherein, prior to (a), said biological sample is stored in said container comprising said formulation for a time period of least 2 days.

124. The method of claim 123, wherein said formulation comprises sodium thioglycolate, sodium phosphate dibasic, sodium chloride, potassium chloride, potassium phosphate monobasic, magnesium sulfate heptahydrate, L-cysteine, glycerol, L-ascorbic acid, glutathione, glutathione peroxidase, peroxidase, superoxide dismutase, catalase, and DL-dithiothreitol.

125. The method of claim 123, further comprising:
(d) identifying at least 20 viable microorganisms.

126. The method of claim 123, further comprising, prior to (b), storing said container at a temperature of less than −70° C.
